(19) United States
(12) Patent Application Publication
(10) Pub. No.: US 2026/0085313 A1
(43) Pub. Date: Mar. 26, 2026

(54) COMPOUNDS AND METHODS FOR MODULATING SMN2

(71) Applicant: Ionis Pharmaceuticals, Inc., Carlsbad, CA (US)

(72) Inventors: Frank Rigo, Carlsbad, CA (US); Thazha P. Prakash, Carlsbad, CA (US); Kar Yun Karen Ling, Carlsbad, CA (US); W. Brad Wan, Fallbrook, CA (US); William John Drury, III, Oceanside, CA (US)

(21) Appl. No.: 19/197,566

(22) Filed: May 2, 2025

Related U.S. Application Data
(63) Continuation of application No. 17/187,018, filed on Feb. 26, 2021, now abandoned.
(60) Provisional application No. 62/983,545, filed on Feb. 28, 2020.

(51) Int. Cl. C12N 15/113

(52) U.S. Cl. CPC C12N 15/113; C12N 2310/11; C12N 2310/315; C12N 2310/321; C12N 2310/322; C12N 2310/3341

(57) ABSTRACT

Provided are compounds, methods, and pharmaceutical compositions for modulating SMN2 RNA and/or protein in a cell or subject. Such compounds, methods, and pharmaceutical compositions are useful to ameliorate at least one symptom of a neurodegenerative disorder. Such symptoms include reduced muscle strength; inability or reduced ability to sit upright, to stand, and/or walk; reduced neuromuscular activity; reduced electrical activity in one or more muscles; reduced respiration; inability or reduced ability to eat, drink, and/or breathe without assistance; loss of weight or reduced weight gain; and/or decreased survival.

### SEQUENCE LISTING

The present application is being filed along with a Sequence Listing in electronic format. The Sequence Listing is provided as a file entitled 2011256-2535_SL.xml, created on Dec. 20, 2024, which is 4,013,476 bytes in size. The information in the electronic format of the sequence listing is incorporated herein by reference in its entirety.

### FIELD

Provided are compounds, methods, and pharmaceutical compositions for modulating SMN2 RNA in a cell or subject. Such compounds, methods, and pharmaceutical compositions are useful to ameliorate at least one symptom of a neurodegenerative disorder. Such symptoms include reduced muscle strength; inability or reduced ability to sit upright, to stand, and/or walk; reduced neuromuscular activity; reduced electrical activity in one or more muscles; reduced respiration; inability or reduced ability to eat, drink, and/or breathe without assistance; loss of weight or reduced weight gain; and/or decreased survival.

### BACKGROUND

Proximal spinal muscular atrophy (SMA) is a genetic neurodegenerative disorder characterized by the loss of spinal motor neurons. SMA is an autosomal recessive disease of early onset and is a leading genetic cause of death among infants. The severity of SMA varies among patients and it has thus been classified into four types. Type I SMA is the most severe form with onset at birth or within 6 months, and typically results in death within 2 years. Children with Type I SMA are unable to sit or walk. Type II SMA is the intermediate form and patients are able to sit, but cannot stand or walk. Patients with Type III SMA, a chronic form of the disease, typically develop SMA after 18 months of age (Lefebvre et al., Hum. Mol. Genet., 1998, 7, 1531-1536). Type IV SMA is a milder form and typically has an onset after 18 years of age, sometimes after 10 years of age; patients with Type IV SMA experience limited mild motor impairment, are able to walk in adulthood and generally do not have respiratory or nutritional problems (Farrar et al., Ann. Neurol., 2017, 81, 355-368; D'Amico et al., Orphanet J. of Rare Diseases, 2011, 6: 71).

The molecular basis of SMA is the loss of both copies of survival motor neuron gene 1 (SMN1), which may also be known as SMN Telomeric, and encodes a protein that is part of a multi-protein complex thought to be involved in snRNP biogenesis and recycling. A nearly identical gene, SMN2, which may also be known as SMN Centromeric, exists in a duplicated region on chromosome Sq13 and modulates disease severity. Although SMN1 and SMN2 have the potential to code for the same protein, expression of the normal SMN1 gene results solely in expression of full-length survival motor neuron (SMN) protein, while expression of the SMN2 gene results in two different protein forms, full-length SMN2 protein, and a truncated SMN2 protein, SMNA7 protein. SMN2 contains a translationally silent mutation at position +6 of exon 7, which results in inefficient inclusion of exon 7 in SMN2 transcripts. Thus, the predominant form of SMN2 is a truncated version, lacking exon 7, which is unstable and inactive (Cartegni and Krainer, Nat. Genet., 2002, 30, 377-384). Expression of the SMN2 gene results in approximately 10-20% of the full-length SMN protein and 80-90% of the unstable/non-functional SMNA7 protein. SMN protein plays a well-established role in assembly of the spliceosome and may also mediate mRNA trafficking in the axon and nerve terminus of neurons.

It is an object herein to provide compounds, methods, and pharmaceutical compositions for the treatment of SMA.

### SUMMARY OF THE INVENTION

Provided herein are compounds, methods, and pharmaceutical compositions for modulating splicing of SMN2 RNA in a cell or subject. In certain embodiments, compounds useful for modulating splicing of SMN2 RNA are oligomeric compounds. In certain embodiments, oligomeric compounds increase the amount of SMN2 RNA including exon 7. In certain embodiments, oligomeric compounds increase full-length SMN2 protein expression. In certain embodiments, the oligomeric compound comprises a modified oligonucleotide. In certain embodiments, the subject has a neurodegenerative disease. In certain embodiments, the subject has Spinal Muscular Atrophy (SMA).

Also provided are methods useful for ameliorating at least one symptom of a neurodegenerative disease. In certain embodiments, the neurodegenerative disease is SMA. In certain embodiments, symptoms include reduced muscle strength; inability or reduced ability to sit upright, to stand, and/or walk; reduced neuromuscular activity; reduced electrical activity in one or more muscles; reduced respiration; inability or reduced ability to eat, drink, and/or breathe without assistance; loss of weight or reduced weight gain; and/or decreased survival. In certain embodiments, provided herein are modified oligonucleotides for treating SMA.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, and GenBank and NCBI reference sequence records are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxyribonucleoside” means a nucleoside comprising a 2′-H(H) deoxyribosyl sugar moiety. In certain embodiments, a 2′-deoxyribonucleoside is a 2′-β-D deoxyribonucleoside and comprises a 2′-β-D-deoxyribosyl sugar moiety, which has the β-D configuration as found in naturally occurring deoxyribonucleic acids (DNA). In certain embodiments, a 2′-deoxyribonucleoside may comprise a modified nucleobase or may comprise an RNA nucleobase (uracil).

As used herein, “2′-MOE” means a 2′-OCH₂CH₂OCH₃ group in place of the 2′—OH group of a ribosyl sugar moiety. A “2′-MOE sugar moiety” is a sugar moiety with a 2′-OCH₂CH₂OCH₃ group in place of the 2′—OH group of a ribosyl sugar moiety. Unless otherwise indicated, a 2′-MOE sugar moiety is in the β-D configuration. “MOE” means O-methoxyethyl.

As used herein, “2′-MOE nucleoside” means a nucleoside comprising a 2′-MOE sugar moiety.

As used herein, “2′-NMA” means a —O—CH₂—C(═O)—NH—CH₃ group in place of the 2′—OH group of a ribosyl sugar moiety. A “2′-NMA sugar moiety” is a sugar moiety with a 2′-O—CH₂—C(═O)—NH—CH₃ group in place of the 2′-OH group of a ribosyl sugar moiety. Unless otherwise indicated, a 2′-NMA sugar moiety is in the β-D configuration. “NMA” means O—N-methyl acetamide.

As used herein, “2′-NMA nucleoside” means a nucleoside comprising a 2′-NMA sugar moiety.

As used herein, “2′-OMe” means a 2′-OCH₃ group in place of the 2′—OH group of a ribosyl sugar moiety. A “2′-OMe sugar moiety” is a sugar moiety with a 2′-OCH₃ group in place of the 2′—OH group of a ribosyl sugar moiety.

Unless otherwise indicated, a 2′-OMe sugar moiety is in the β-D configuration. “OMe” means O-methyl.

As used herein, “2′-OMe nucleoside” means a nucleoside comprising a 2′-OMe sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2-substituent group other than H or OH.

As used herein, “5-methyl cytosine” means a cytosine modified with a methyl group attached to the 5 position.

A 5-methyl cytosine is a modified nucleobase.

As used herein, “administering” means providing a pharmaceutical agent to a subject.

As used herein, “ameliorate” in reference to a treatment means improvement in at least one symptom relative to the same symptom in the absence of the treatment. In certain embodiments, amelioration is the reduction in the severity or frequency of a symptom, or the delayed onset or slowing of progression in the severity or frequency of a symptom. In certain embodiments, the symptom is reduced muscle strength; inability or reduced ability to sit upright, to stand, and/or walk; reduced neuromuscular activity; reduced electrical activity in one or more muscles; reduced respiration; inability or reduced ability to eat, drink, and/or breathe without assistance; loss of weight or reduced weight gain; and/or decreased survival.

As used herein, “antisense activity” means any detectable and/or measurable change attributable to the hybridization of an antisense compound to its target nucleic acid.

As used herein, “antisense compound” means an oligomeric compound or oligomeric duplex capable of achieving at least one antisense activity.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the furanosyl moiety is a ribosyl moiety. In certain embodiments, the bicyclic sugar moiety does not comprise a furanosyl moiety.

As used herein, “cerebrospinal fluid” or “CSF” means the fluid filling the space around the brain and spinal cord. “Artificial cerebrospinal fluid” or “aCSF” means a prepared or manufactured fluid that has certain properties of cerebrospinal fluid.

As used herein, “cEt” means a 4′ to 2′ bridge in place of the 2′OH-group of a ribosyl sugar moiety, wherein the bridge has the formula of 4′-CH(CH₃)—O-2′, and wherein the methyl group of the bridge is in the S configuration. A “cEt sugar moiety” is a bicyclic sugar moiety with a 4′ to 2′ bridge in place of the 2′OH-group of a ribosyl sugar moiety, wherein the bridge has the formula of 4′-CH(CH₃)—O-2′, and wherein the methyl group of the bridge is in the S configuration. “cEt” means constrained ethyl.

As used herein, “cEt nucleoside” means a nucleoside comprising a cEt sugar moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “complementary” in reference to an oligonucleotide means that at least 70% of the nucleobases of the oligonucleotide or one or more portions thereof and the nucleobases of another nucleic acid or one or more portions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) with thymine (T), adenine (A) with uracil (U), cytosine (C) with guanine (G), and 5-methyl cytosine (mC) with guanine (G). Complementary oligonucleotides and/or target nucleic acids need not have nucleobase complementarity at each nucleoside. Rather, some mismatches are tolerated. As used herein, “fully complementary” or “100% complementary” in reference to an oligonucleotide, or a portion thereof, means that the oligonucleotide, or portion thereof, is complementary to another oligonucleotide or target nucleic acid at each nucleobase of the shorter of the two oligonucleotides, or at each nucleoside if the oligonucleotides are the same length.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “internucleoside linkage” means the covalent linkage between contiguous nucleosides in an oligonucleotide. As used herein, “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “mismatch” or “non-complementary” means a nucleobase of a first oligonucleotide that is not complementary with the corresponding nucleobase of a second oligonucleotide or target nucleic acid when the first and second oligonucleotide are aligned.

As used herein, “motif” means the pattern of unmodified and/or modified sugar moieties, nucleobases, and/or internucleoside linkages, in an oligonucleotide.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. As used herein, “nucleobase sequence” means the order of contiguous nucleobases in a target nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. “Linked nucleosides” are nucleosides that are connected in a contiguous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutical composition” means a mixture of substances suitable for administering to a subject. For example, a pharmaceutical composition may comprise an oligomeric compound and a sterile aqueous solution.

As used herein, “pharmaceutically acceptable carrier or diluent” means any substance suitable for use in administering to a subject. Certain such carriers enable pharmaceutical compositions to be formulated as, for example, tablets, pills, dragees, capsules, liquids, gels, syrups, slurries, suspension, and lozenges for the oral ingestion by a subject. In certain embodiments, a pharmaceutically acceptable carrier or diluent is sterile water, sterile saline, sterile buffer solution, or sterile artificial cerebrospinal fluid.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “RNA” means an RNA transcript and includes pre-mRNA and mature mRNA unless otherwise specified.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “subject” means a human or non-human animal.

As used herein, “sugar moiety” means an unmodified sugar moiety or a modified sugar moiety. As used herein, “unmodified sugar moiety” means a 2′-OH(H) β-D ribosyl moiety, as found in RNA (an “unmodified RNA sugar moiety”), or a 2′-H(H) β-D deoxyribosyl moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “standard in vivo assay” means the assay described in Example 2 and reasonable variations thereof.

As used herein, “symptom” means any physical feature or test result that indicates the existence or extent of a disease or disorder. In certain embodiments, a symptom is apparent to a subject or to a medical professional examining or testing the subject.

As used herein, “target nucleic acid” means a nucleic acid that an antisense compound is designed to affect.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to a subject. For example, a therapeutically effective amount improves a symptom of a disease.

### CERTAIN EMBODIMENTS

The present disclosure provides the following non-limiting numbered embodiments:

Embodiment 1. An oligomeric compound comprising a modified oligonucleotide consisting of 16, 17, 18, 19, or 20 linked nucleosides and having a nucleobase sequence comprising at least 15 or at least 16 contiguous nucleobases of any of the nucleobase sequences of SEQ ID NOs: 20-50, wherein the modified oligonucleotide comprises at least one modification selected from a modified sugar moiety and a modified internucleoside linkage.

Embodiment 2. An oligomeric compound comprising a modified oligonucleotide consisting of 17, 18, 19, or 20 linked nucleosides and having a nucleobase sequence comprising at least 15, at least 16, or at least 17 contiguous nucleobases of any of the nucleobase sequences of SEQ ID NOs: 20-27, 29-30, or 32-50, wherein the modified oligonucleotide comprises at least one modification selected from a modified sugar moiety and a modified internucleoside linkage.

Embodiment 3. An oligomeric compound comprising a modified oligonucleotide consisting of 18, 19, or 20 linked nucleosides and having a nucleobase sequence comprising at least 15, at least 16, or at least 17, or at least 18 contiguous nucleobases of any of the nucleobase sequences of SEQ ID NOs: 20-27, 30, or 33-50, wherein the modified oligonucleotide comprises at least one modification selected from a modified sugar moiety and a modified internucleoside linkage.

Embodiment 4. An oligomeric compound comprising a modified oligonucleotide consisting of 19 or 20 linked nucleosides and having a nucleobase sequence comprising at least 15, at least 16, or at least 17, at least 18, or at least 19 contiguous nucleobases of any of the nucleobase sequences of SEQ ID NOs: 20, 22, 24-27, 30, 33-50, wherein the modified oligonucleotide comprises at least one modification selected from a modified sugar moiety and a modified internucleoside linkage.

Embodiment 5. An oligomeric compound comprising a modified oligonucleotide consisting of 20 linked nucleosides and having a nucleobase sequence comprising at least 15, at least 16, or at least 17, at least 18, at least 19, or at least 20 contiguous nucleobases of any of the nucleobase sequences of SEQ ID NOs: 20, 22, 25, 27, 35, 39-46, or 49, wherein the modified oligonucleotide comprises at least one modification selected from a modified sugar moiety and a modified internucleoside linkage.

Embodiment 6. The oligomeric compound of any of embodiments 1-5, wherein the modified oligonucleotide has a nucleobase sequence that is at least 80%, 85%, 87.5%, 88.2%, 89%, 89.4%, 90%, 93.7%, 94%, 94.7%, 95%, or is 100% complementary to the nucleobase sequence of SEQ ID NO: 1 when measured across the entire nucleobase sequence of the modified oligonucleotide.

Embodiment 7. The oligomeric compound of any of embodiments 1-6, wherein the modified oligonucleotide has an internucleoside linkage motif (5′ to 3′) selected from: sososssssssssssss, ssosssssssssssoss, ssosssssosssssoss, ssosssosssosssoss, soossssssssssooss, sooosssssssssooss, sooossssssssoooss, sssssssooosssssss, ssossssssssssssss, ssssossssssssssss, ssssssossssssssss, ssssssssossssssss, ssssssssssossssss, ssssssssssssossss, ssssssssssssssoss, sossssssssssssoss, sosssssssssosssss, sosssssssosssssss, sosssssosssssssss, sosssosssssssssss, ssssosssssssssoss, ssssssosssssssoss, ssssssssosssssoss, ssssssssssosssoss, ssssssssssssososs, soossssssssssssss, sssoossssssssssss, sssssoossssssssss, sssssssoossssssss, sssssssssoossssss, sssssssssssoossss, sssssssssssssooss, sssssssoooossssss, ssoooosssssssssss, ssssoooosssssssss, ssssssssoooosssss, ssssssssssoooosss, sssssssssssooooss, ssssssooooossssss, ssssssoooooosssss, soooosssssssoooss, sssssooooooosssss, ssssssssssssssoss, ssssssssssssosss, sssssssssssssooss, ssssssssssssososs, sssssssssssosssss, sssssssssssososss, ssssssssssossosss, sssssssssosssssss, sssssssssosssosss, ssssssssosssssoss, ssssssssossssosss, sssssssosssssssss, sssssssoossssssss, sssssosssssssssss, sssosssssssssssss, sosssssssssssssss, sossssssossssssss, soossssssssssssss, ossssssssssssssssso, sssssssssssssssssoo, ssssssssssssssssoss, sssssssssssssssooss, ssssssssssssssososs, sssssssssosssssssss, sssssssssossssssoss, ssssssssoosssssssss, sosssssssssssssssss, sossssssssssssssoss, sosssssssosssssssss, sososssssssssssssss, soossssssssssssssss, sssssssssssssssssss, ssssssssssssssssso, ossssssssssssssssss, ssssssssssssososso, sssssssssssssssoss, sssssssssssssososs, ssssssssosssssssss, ssssssssossssssoss, sossssssssssssssss, sosssssssssssssoss, sossssssosssssssss, sosossssssssssssss, ssssssssssooooss, ssssssssoooossss, sssssssooossssss, ssssssoooossssss, ssssssooooosssss, sssssoooooosssss, sssssooooooossss, ssssoooossssssss, ssossssssssssoss, ssosssssossssoss, ssosssosssossoss, ssossossossososs, ssososososososss, ssoooossssssssss, soosssssssssooss, sooossssssssooss, sooosssssssoooss, soooossssssoooss, sssssssssooooss, ssssssssoooosss, ssssssooossssss, ssssssoooosssss, sssssooooosssss, sssssoooooossss, ssssoooosssssss, ssssooooooossss, sssosssosssosss, ssosssssssssoss, ssossossossosss, ssossossosososs, ssososososososs, ssoooosssssssss, soossssssssooss, sooosssssssooss, sooossssssoooss, and soooosssssoooss; wherein ‘s’ represents a phosphorothioate internucleoside linkage and ‘o’ represents a phosphodiester internucleoside linkage.

Embodiment 8. The oligomeric compound of any of embodiments 1-6, wherein the modified oligonucleotide has an internucleoside linkage motif selected from: sssssssssssssssxs and ssssssssssssssssx, wherein ‘s’ represents a phosphorothioate internucleoside linkage, ‘o’ represents a phosphodiester internucleoside linkage, and “x” represents a methoxypropylphosphonate internucleoside linkage.

Embodiment 9. The oligomeric compound of any of embodiments 1-6, wherein the modified oligonucleotide has an internucleoside linkage motif selected from: zzzzzzzzzzzzzzzzz, ssssssssssszzzzzz, ssssszzzzzzssssss, zzooooooooooooozz, zzzzooooooooooozz, zzzzzzooooooooozz, zzzzzzzzooooooozz, and ssooooooooooooss, wherein ‘s’ represents a phosphorothioate internucleoside linkage, ‘o’ represents a phosphodiester internucleoside linkage, and “z” represents a mesyl phosphoramidate internucleoside linkage.

Embodiment 10. The oligomeric compound of any of embodiments 1-9, wherein the modified oligonucleotide has a sugar motif (5′ to 3′) selected from: eeeeeeeeeeeeeeeeeeee, eeeeeeeeeeeeeeeeeee, eeeeeeeeeeeeeeeeee, eeeeeeeeeeeeeeeee, eeeeeeeeeeeeeeee, nnnnnnnnnnnnnnnn, nnnnnnnnnnnnnnnnn, nnnnnnnnnnnnnnnnnn, nnnnnnnnnnnnnnnnnnn, nnnnnnnnnnnnnnnnnnnn, nennnnneneennnnnnn, nnnnnnnnnnnnenneen, nennnnneneenenneen, nnnnnnnnnnnnnnnnnne, nnnnnnnnnnnnnnnnnnd, nnnnnnnnnnnnnnnnnny, nnnnnnnnnnnnnnnnnndd, nnnnnnnnnnnnnnnnnned, nnnnnnnnnnnnnnnnnnde, nnnnnnnnnnnnnnnnnnee, eeeeeeeeeeeeeeeeeedd, eeeeeeeeeeeeeeeeeeed, eeeeeeeeeeeeeeeeeede, nnnnnnnnnnnnnnnnnnd, nnnnnnnnnnnnnnnnnne, eeeeeeeeeeeeeeeeeed, keekeekeekeekeeeek, keeekeeekeeekeeeek, keeeeekeeeeekeeeek, keeeeeeekeeeeeeeek, keeeeeeeeeeeeeeeek, eeekeekeekeekeekek, eeekeekeekeekeekee, eeeeeekeekeekeekee, eeeeeekeekeekeeeee, eeeeeekeeeeekeeeee, keekeekeekeeeeeeee, eeeeeeeekeekeekeek, keekeekeeeeeeeeeee, eeeeeeeeeeekeekeek, keekeeeeeeeeeeeeee, eeeeeeeeeeeeeekeek, keekeekeekeekeeek, keeekeeekeeekeeek, keeeekeeeeekeeeek, keeeeeeekeeeeeeek, keeeeeeeeeeeeeeek, eekeekeekeekeekek, eekeekeekeekeekee, eeeeekeekeekeekee, eeeeekeekeekeeeee, eeeeekeeeeekeeeee, keekeekeekeeeeeee, eeeeeeekeekeekeek, keekeekeeeeeeeeee, eeeeeeeeeekeekeek, keekeeeeeeeeeeeee, eeeeeeeeeeeeekeek, keekeekeekeekeek, keeekeeekeeekeek, keeeekeeeekeeeek, keeeeeeekeeeeeek, keeeeeeeeeeeeeek, kekeekeekeekeeke, eekeekeekeekeeke, eeeeekeekeekeeke, eeeeekeekeekeeee, eeeeekeeeeekeeee, keekeekeekeeeeee, eeeeeekeekeekeek, keekeekeeeeeeeee, eeeeeeeeekeekeek, keekeeeeeeeeeeee, eeeeeeeeeeeekeek, eeeeeeeeeeeeeeeeeed, eeeeeeeeeeeeeeeeeey, ennnnnnnnnnnnnnnnnn, and ennnnnnnnnnnnnnnnnne; wherein ‘e’ represents a 2′-MOE sugar moiety, ‘n’ represents a 2′-NMA sugar moiety, ‘k’ represents a cEt sugar moiety, ‘d’ represents a 2′-β-D-deoxyribosyl sugar moiety, and ‘y’ represents a 2′-OMe sugar moiety.

Embodiment 11. The oligomeric compound of any of embodiments 1-9, wherein the modified oligonucleotide has a sugar motif (5′ to 3′) selected from nnnnnnnnnnnnnnnenn and nnnnnnnnnnnnnnnnen, wherein ‘e’ represents a 2′-MOE sugar moiety and ‘n’ represents a 2′-NMA sugar moiety.

Embodiment 12. The oligomeric compound of any of embodiments 1-9, wherein the modified oligonucleotide has a sugar motif (5′ to 3′) of qqnqqqqqnqnnqnqqnn, wherein in each ‘n’ represents a 2′-NMA sugar moiety, and each ‘q’ is independently selected from a 2′-O—(N,N-dimethyl) acetamide sugar moiety, a 2′-O—(N-ethyl) acetamide sugar moiety, a 2′-O—(N-propyl) acetamide sugar moiety, a 2′O—(N-cyclopropyl) acetamide sugar moiety, and a 2′-O—(N-cyclopropylmethyl) acetamide sugar moiety.

Embodiment 13. The oligomeric compound of any of embodiments 1-9, wherein the modified oligonucleotide comprises at least one modified sugar moiety.

Embodiment 14. The oligomeric compound of embodiment 13, wherein the modified oligonucleotide comprises at least one bicyclic sugar moiety.

Embodiment 15. The oligomeric compound of embodiment 14, wherein the bicyclic sugar moiety has a 4′-2′ bridge, wherein the 4′-2′ bridge is selected from —CH₂—O—; and —CH(CH₃)—O—.

Embodiment 16. The oligomeric compound of embodiment 13, wherein the modified oligonucleotide comprises at least one non-bicyclic modified sugar moiety.

Embodiment 17. The oligomeric compound of embodiment 16, wherein the non-bicyclic modified sugar moiety is any of a 2′-MOE sugar moiety, a 2′-NMA sugar moiety, a 2′-OMe sugar moiety, or a 2′-F sugar moiety.

Embodiment 18. The oligomeric compound of embodiment 13, wherein the modified oligonucleotide comprises at least one sugar surrogate.

Embodiment 19. The oligomeric compound of embodiment 18, wherein the sugar surrogate is any of morpholino, modified morpholino, PNA, THP, and F-HNA.

Embodiment 20. The oligomeric compound of any of embodiments 1-6 and 10-19, wherein the modified oligonucleotide comprises at least one modified internucleoside linkage.

Embodiment 21. The oligomeric compound of embodiment 20, wherein each internucleoside linkage of the modified oligonucleotide is a modified internucleoside linkage.

Embodiment 22. The oligomeric compound of embodiment 20 or embodiment 21, wherein the modified internucleoside linkage is a phosphorothioate internucleoside linkage.

Embodiment 23. The oligomeric compound of any of embodiments 1-20 or 22, wherein the modified oligonucleotide comprises at least one phosphodiester internucleoside linkage.

Embodiment 24. The oligomeric compound of any of embodiments 20, 22, or 23, wherein each internucleoside linkage is independently selected from a phosphodiester internucleoside linkage and a phosphorothioate internucleoside linkage.

Embodiment 25. The oligomeric compound of any of embodiments 13-19, wherein the modified oligonucleotide has an internucleoside linkage motif (5′ to 3′) selected from: sososssssssssssss, soossssssssssssss, sosssosssssssssss, sosssssosssssssss, sosssssssosssssss, sssoossssssssssss, sssssssoossssssss, sssssssssoossssss, and sssssssssssoossss; wherein ‘s’ represents a phosphorothioate internucleoside linkage and ‘o’ represents a phosphodiester internucleoside linkage.

Embodiment 26. The oligomeric compound of any of embodiments 1-25, wherein the modified oligonucleotide comprises a modified nucleobase.

Embodiment 27. The oligomeric compound of embodiment 26, wherein the modified nucleobase is a 5-methyl cytosine.

Embodiment 28. The oligomeric compound of any of embodiments 1-27, wherein the modified oligonucleotide consists of 16, 17, 18, 19, or 20 linked nucleosides.

Embodiment 29. The oligomeric compound of any of embodiments 1-28, wherein the modified oligonucleotide comprises 1 or 2 non-complementary nucleobases.

Embodiment 30. The oligomeric compound of any of embodiments 1-29, wherein the modified oligonucleotide comprises 1 or 2 cleavable moieties.

Embodiment 31. The oligomeric compound of embodiment 30, wherein the cleavable moiety is a phophodiester internucleoside linkage.

Embodiment 32. The oligomeric compound of any of embodiments 1-31, consisting of the modified oligonucleotide.

Embodiment 33. The oligomeric compound of any of embodiments 1-32, wherein the oligomeric compound is a singled-stranded oligomeric compound.

Embodiment 34. An oligomeric compound comprising a modified oligonucleotide according to the following chemical notation: ^{m}Cₑₛ Aₑₒ ^{m}Cₑₛ Tₑₒ Tₑₛ Tₑₛ ^{m}Cₑₛ Aₑₛ Tₑₛ Aₑₛ Aₑₛ Tₑₛ Gₑₛ^{m}Cₑₛ Tₑₛ Gₑₛ Gₑₛ ^{m}Cₑ (SEQ ID NO: 21), wherein:
A=an adenine nucleobase,
^{m}C=a 5-methyl cytosine nucleobase,
G=a guanine nucleobase,
T=a thymine nucleobase,
e=a 2′-MOE sugar moiety,
s=a phosphorothioate internucleoside linkage, and
o=a phosphodiester internucleoside linkage.

Embodiment 35. An oligomeric compound comprising a modified oligonucleotide according to the following chemical notation: Tₑₒ Tₑₛ ^{m}Cₑₛ Aₑₛ ^{m}Cₑₛ Tₑₛ Tₑₛ Tₑₛ ^{m}Cₑₛ Aₑₛ Tₑₛ Aₑₛ Aₑₛ Tₑₛ Gₑₛ ^{m}Cₑₛ Tₑₛ Gₑₛ Gₑₒ ^{m}Cₑ (SEQ ID NO: 22), wherein:
A=an adenine nucleobase,
^{m}C=a 5-methyl cytosine nucleobase,
G=a guanine nucleobase,
T=a thymine nucleobase,
e=a 2′-MOE sugar moiety,
s=a phosphorothioate internucleoside linkage, and
o=a phosphodiester internucleoside linkage.

Embodiment 36. An oligomeric compound comprising a modified oligonucleotide according to the following chemical notation: Tₑₒ Tₙₛ ^{m}Cₙₛ Aₙₛ ^{m}Cₙₛ Tₙₛ Tₙₛ Tₙₛ ^{m}Cₙₛ Aₙₛ Tₙₛ Gₙₛ ^{m}Cₙₛ Tₙₛ Gₙₛ Gₙₒ ^{m}Cₑ (SEQ ID NO: 51), wherein:
A=an adenine nucleobase,
^{m}C=a 5-methyl cytosine nucleobase,
G=a guanine nucleobase,
T=a thymine nucleobase,
e=a 2′-MOE sugar moiety,
n=a 2′-NMA sugar moiety,
s=a phosphorothioate internucleoside linkage, and
o=a phosphodiester internucleoside linkage.

Embodiment 37. An oligomeric compound comprising a modified oligonucleotide according to the following chemical notation: ^{m}Cₙₛ Aₙₒ ^{m}Cₙₛ Tₙₒ Tₙₛ Tₙₛ ^{m}Cₙₛ Aₙₛ Tₙₛ Aₙₛ Aₙₛ Tₙₛ Gₙₛ ^{m}Cₙₛ Tₙₛ Gₙₛ Gₙₛ ^{m}Cₙ (SEQ ID NO: 52), wherein:
A=an adenine nucleobase,
^{m}C=a 5-methyl cytosine nucleobase,
G=a guanine nucleobase,
T=a thymine nucleobase,
n=a 2′-NMA sugar moiety,
s=a phosphorothioate internucleoside linkage, and
o=a phosphodiester internucleoside linkage.

Embodiment 38. A modified oligonucleotide according to the following chemical structure:

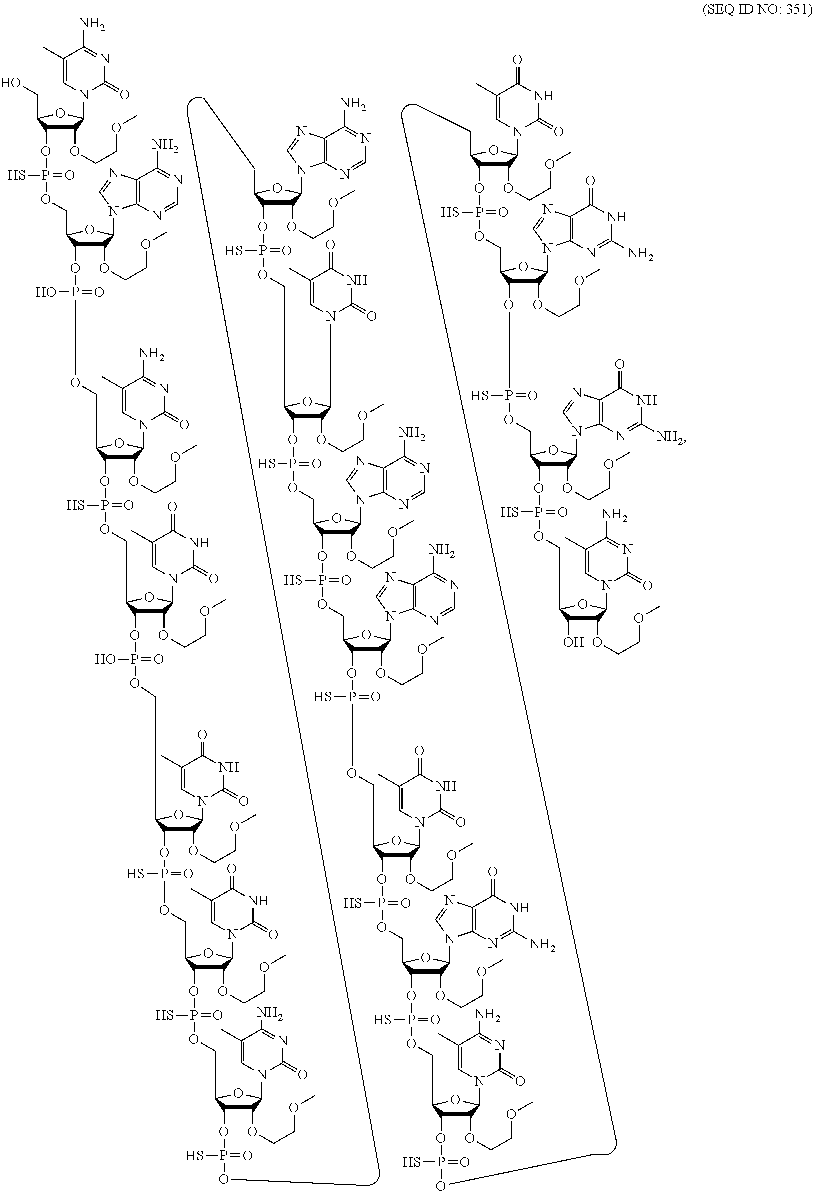

or a salt thereof.

Embodiment 39. The modified oligonucleotide of embodiment 38, which is the sodium salt or the potassium salt.

Embodiment 40. A modified oligonucleotide according to the following chemical structure:

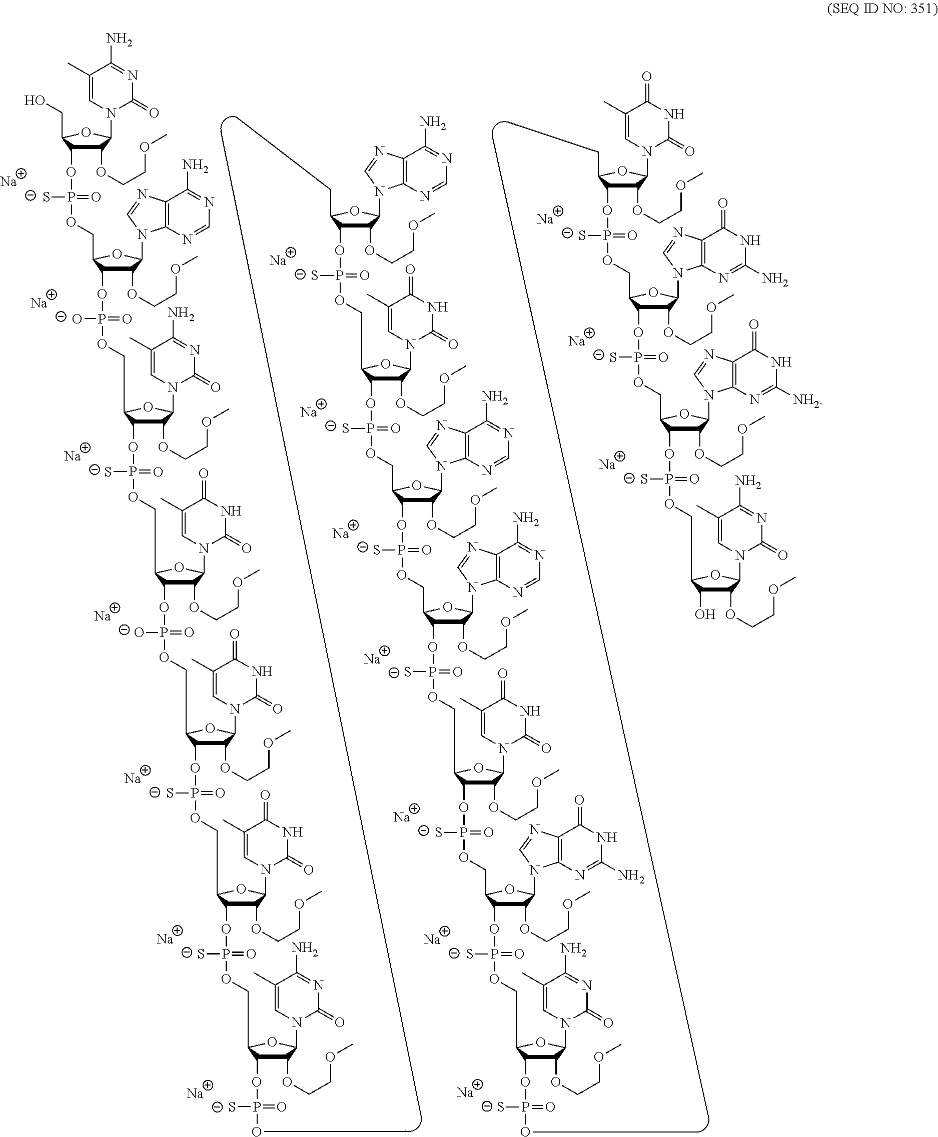

Embodiment 41. A modified oligonucleotide according to the following chemical structure:

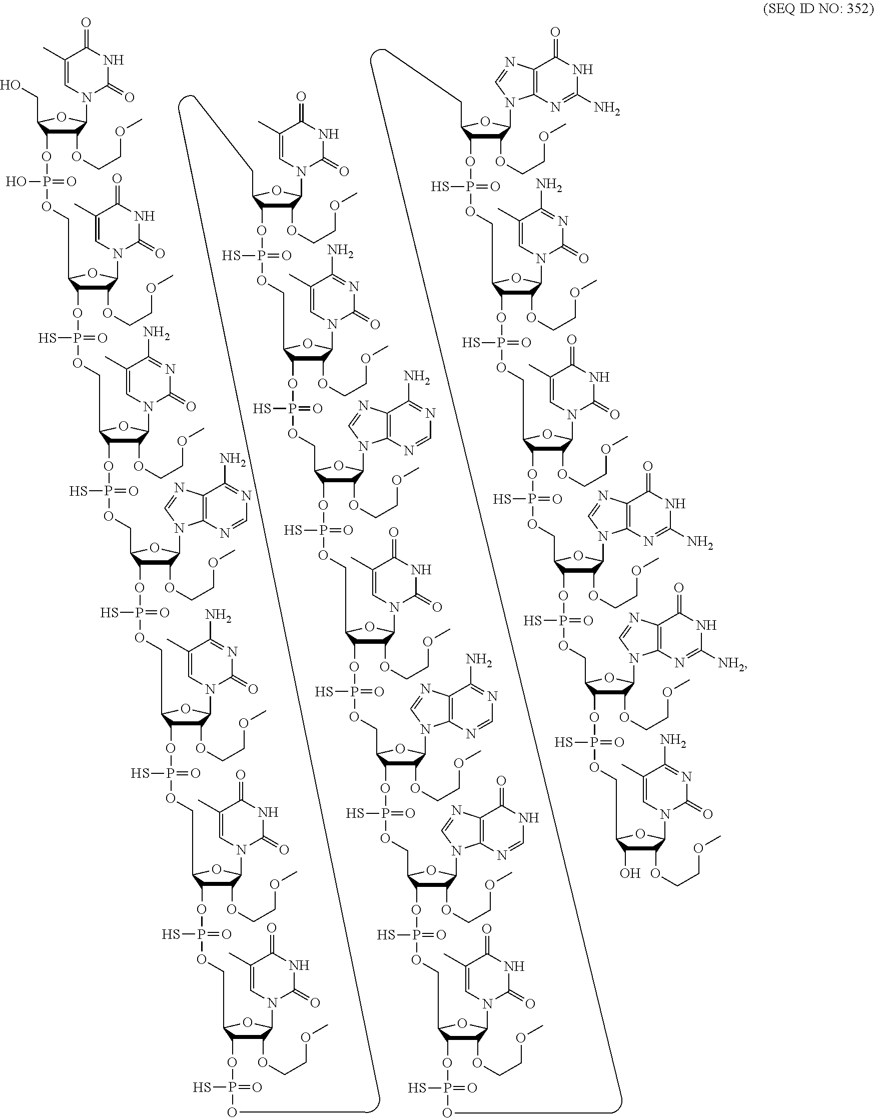

or a salt thereof.

Embodiment 42. The modified oligonucleotide of embodiment 41, which is the sodium salt or the potassium salt.

Embodiment 43. A modified oligonucleotide according to the following chemical structure:

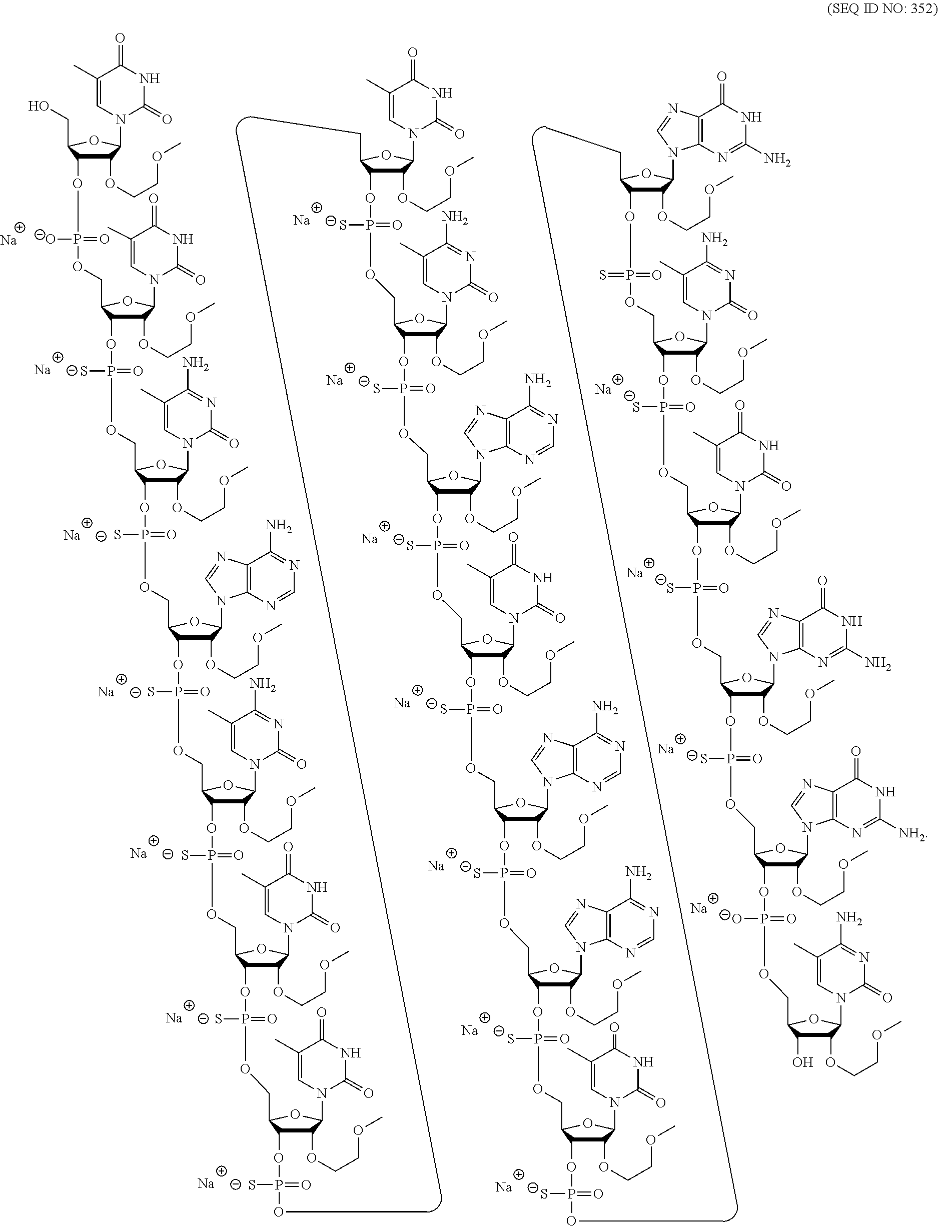

Embodiment 44. A modified oligonucleotide according to the following chemical structure:

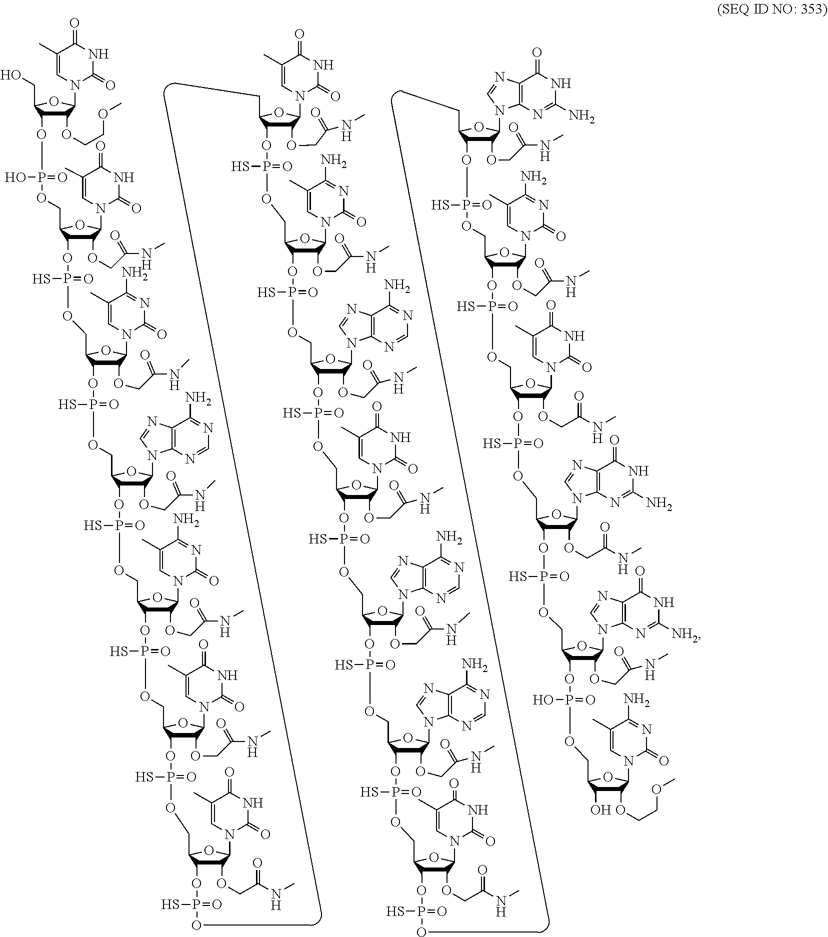

or a salt thereof.

Embodiment 45. The modified oligonucleotide of embodiment 44, which is the sodium salt or the potassium salt.

Embodiment 46. A modified oligonucleotide corresponding to the following chemical structure:

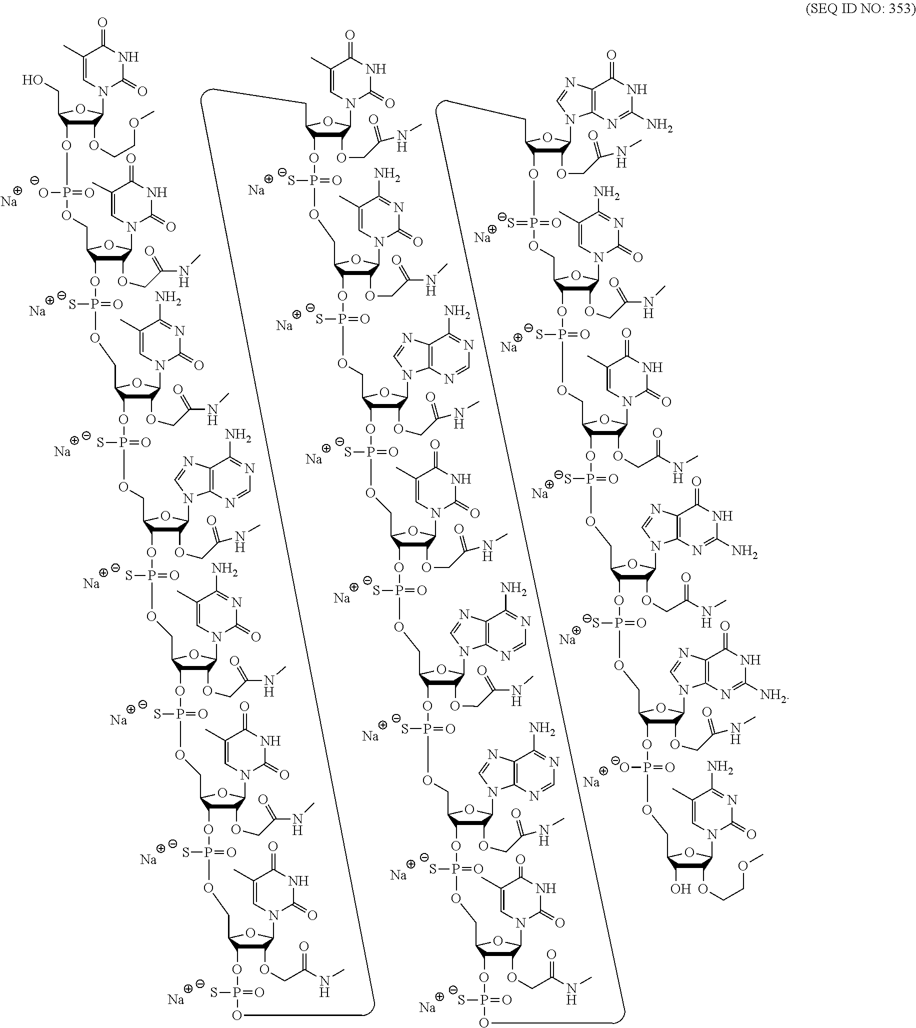

Embodiment 47. A modified oligonucleotide according to the following chemical structure:

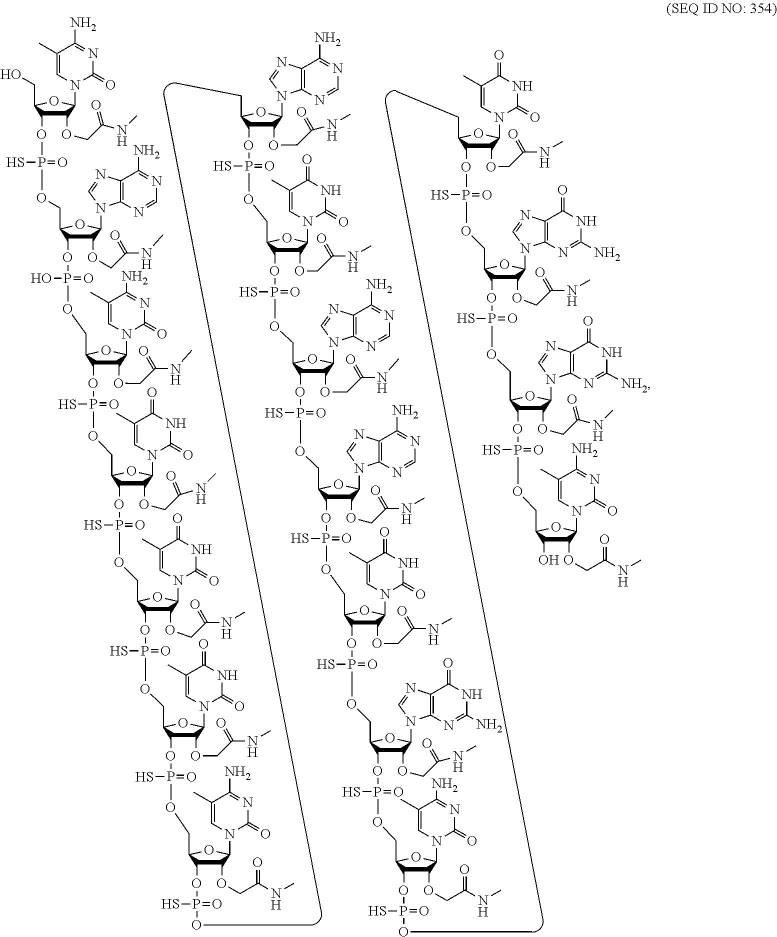

or a salt thereof.

Embodiment 48. The modified oligonucleotide of embodiment 47, which is the sodium salt or the potassium salt.

Embodiment 49. A modified oligonucleotide according to the following chemical structure:

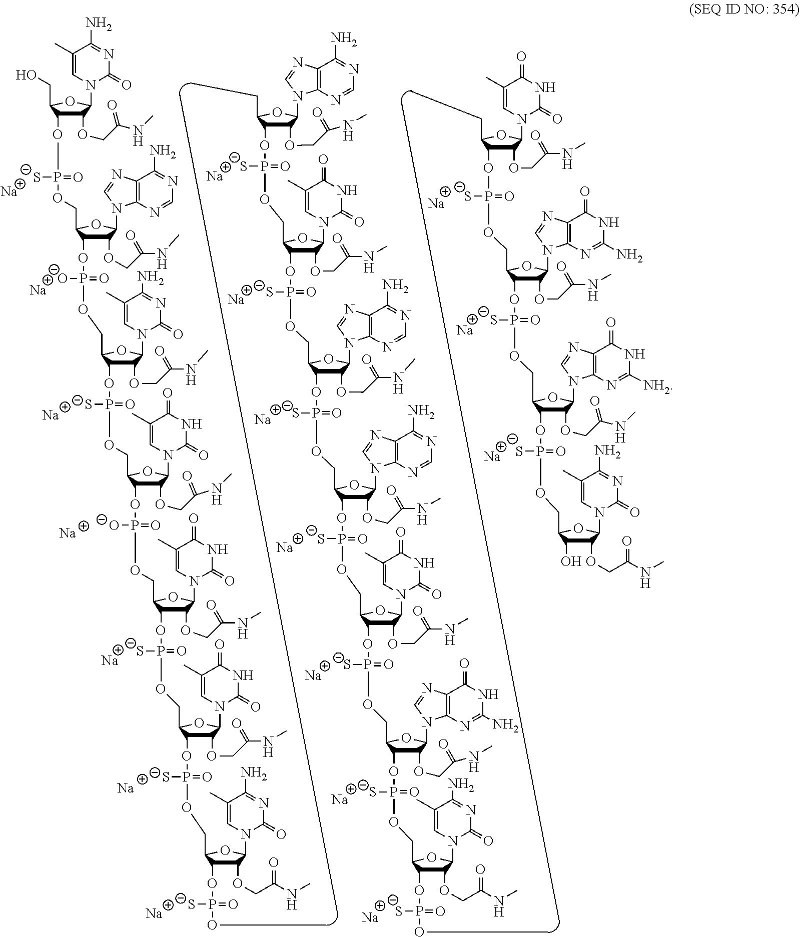

Embodiment 50. A pharmaceutical composition comprising the oligomeric compound of any of embodiments 1-36 or the modified oligonucleotide of any of embodiments 38-49, and a pharmaceutically acceptable diluent or carrier.

Embodiment 51. The pharmaceutical composition of embodiment 50, comprising a pharmaceutically acceptable diluent and wherein the pharmaceutically acceptable diluent is artificial CSF (aCSF) or PBS.

Embodiment 52. The pharmaceutical composition of embodiment 51, wherein the pharmaceutical composition consists essentially of the modified oligonucleotide and artificial CSF (aCSF).

Embodiment 53. The pharmaceutical composition of embodiment 51, wherein the pharmaceutical composition consists essentially of the modified oligonucleotide and PBS.

Embodiment 54. A chirally enriched population of modified oligonucleotides of any of embodiments 38-49, wherein the population is enriched for modified oligonucleotides comprising at least one particular phosphorothioate internucleoside linkage having a particular stereochemical configuration.

Embodiment 55. The chirally enriched population of embodiment 54, wherein the population is enriched for modified oligonucleotides comprising at least one particular phosphorothioate internucleoside linkage having the (Sp) configuration.

Embodiment 56. The chirally enriched population of embodiment 54, wherein the population is enriched for modified oligonucleotides comprising at least one particular phosphorothioate internucleoside linkage having the (Rp) configuration.

Embodiment 57. The chirally enriched population of embodiment 54, wherein the population is enriched for modified oligonucleotides having a particular, independently selected stereochemical configuration at each phosphorothioate internucleoside linkage.

Embodiment 58. The chirally enriched population of embodiment 57, wherein the population is enriched for modified oligonucleotides having the (Sp) configuration at each phosphorothioate internucleoside linkage or for modified oligonucleotides having the (Rp) configuration at each phosphorothioate internucleoside linkage.

Embodiment 59. The chirally enriched population of embodiment 57, wherein the population is enriched for modified oligonucleotides having the (Rp) configuration at one particular phosphorothioate internucleoside linkage and the (Sp) configuration at each of the remaining phosphorothioate internucleoside linkages.

Embodiment 60. The chirally enriched population of embodiment 57, wherein the population is enriched for modified oligonucleotides having at least 3 contiguous phosphorothioate internucleoside linkages in the Sp, Sp, and Rp configurations, in the 5′ to 3′ direction.

Embodiment 61. A population of modified oligonucleotides of any of embodiments 38-49, wherein all of the phosphorothioate internucleoside linkages of the modified oligonucleotide are stereorandom.

Embodiment 62. A method of treating a disease associated with SMN1 or SMN2 comprising administering to a subject having or at risk for developing a disease associated with SMN1 or SMN2 a therapeutically effective amount of a pharmaceutical composition according to any of embodiments 50-53; and thereby treating the disease associated with SMN1 or SMN2.

Embodiment 63. The method of embodiment 62, wherein the disease associated with SMN1 or SMN2 is a neurodegenerative disease.

Embodiment 64. The method of embodiment 63, wherein the neurodegenerative disease is Spinal Muscular Atrophy (SMA).

Embodiment 65. The method of embodiment 64, wherein the SMA is any of Type I SMA, Type II SMA, Type III SMA, or Type IV SMA.

Embodiment 66. The method of embodiment 64 or embodiment 65, wherein at least one symptom of SMA is ameliorated.

Embodiment 67. The method of embodiment 66, wherein the symptom is any of reduced muscle strength; inability or reduced ability to sit upright, to stand, and/or walk; reduced neuromuscular activity; reduced electrical activity in one or more muscles; reduced respiration; inability or reduced ability to eat, drink, and/or breathe without assistance; loss of weight or reduced weight gain; and/or decreased survival.

Embodiment 68. The method of any of embodiments 62-67, wherein the pharmaceutical composition is administered to the central nervous system or systemically.

Embodiment 69. The method of embodiment 68, wherein the pharmaceutical composition is administered to the central nervous system and systemically.

Embodiment 70. The method of any of embodiment 62-67, wherein the pharmaceutical composition is administered any of intrathecally, systemically, subcutaneously, or intramuscularly.

Embodiment 71. A method of increasing SMN2 RNA including exon 7 comprising contacting a cell, tissue, or organ with an oligomeric compound of any of embodiments 1-37, a modified oligonucleotide of any of embodiments 38-49, or a pharmaceutical composition of any of embodiments 50-53.

### Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

### Certain Modified Nucleosides

Modified nucleosides comprise a modified sugar moiety or a modified nucleobase or both a modified sugar moiety and a modified nucleobase.

### Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE” or “O-methoxyethyl”), and 2′-O—N-alkyl acetamide, e.g., 2′-O—N-methyl acetamide (“NMA”), 2′-O—N-dimethyl acetamide, 2′-O—N-ethyl acetamide, or 2′-O—N-propyl acetamide. For example, see U.S. Pat. No. 6,147,200, Prakash et al., 2003, Org. Lett., 5, 403-6. A “2′-O—N-methyl acetamide nucleoside” or “2′-NMA nucleoside” is shown below:

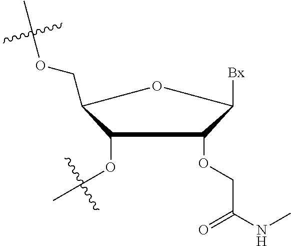

In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂), ON(CH₃)₂, O(CH₂)₂O(CH₂)₂N(CH₃)₂, and N-substituted acetamide (OCH₂C(═O)—N(Rₘ)(Rₙ)), where each Rₘ and R. is, independently, H, an amino protecting group, or substituted or unsubstituted C₁-C₁₀ alkyl, e.g., for example, OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, OCH₂CH₂OCH₃, and OCH₂C(═O)—N(H)CH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙO—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

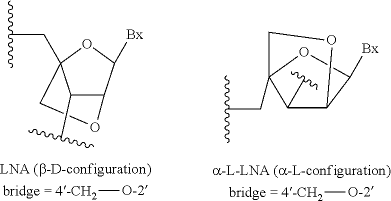

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the j-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

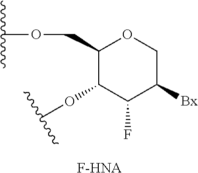

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3-fluoro tetrahydropyran), and nucleosides comprising additional modified THP compounds having the formula:

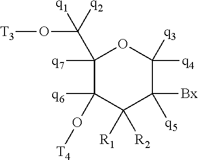

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H.

In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

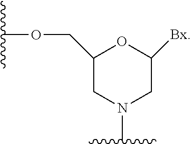

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

### Certain Modified Nucleobases

In certain embodiments, modified oligonucleotides comprise one or more nucleosides comprising an unmodified nucleobase. In certain embodiments, modified oligonucleotides comprise one or more nucleosides comprising a modified nucleobase. In certain embodiments, modified oligonucleotides comprise one or more nucleosides that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyl adenine, 2-thiouracil, 2-thiothymine and 2-thiocytosine, 5-propynyl (—CDC-CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. J., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

### Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphodiesters, which contain a phosphodiester bond, P(O₂)═O, (also referred to as unmodified or naturally occurring linkages); phosphotriesters; methylphosphonates; methoxypropylphosphonates (“MOP”); phosphoramidates; mesyl phosphoramidates; phosphorothioates (P(O₂)═S); and phosphorodithioates (HS—P═S). Representative non-phosphorus containing internucleoside linking groups include but are not limited to methylenemethylimino (—CH₂—N(CH₃)—O—CH₂—); thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate internucleoside linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate internucleoside linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate internucleoside linkage is present in at least 65% of the molecules in the population. In certain embodiments, the particular configuration of the particular phosphorothioate internucleoside linkage is present in at least 70% of the molecules in the population. In certain embodiments, the particular configuration of the particular phosphorothioate internucleoside linkage is present in at least 80% of the molecules in the population. In certain embodiments, the particular configuration of the particular phosphorothioate internucleoside linkage is present in at least 90% of the molecules in the population. In certain embodiments, the particular configuration of the particular phosphorothioate internucleoside linkage is present in at least 99% of the molecules in the population. Such chirally enriched populations of modified oligonucleotides can be generated using synthetic methods known in the art, e.g., methods described in Oka et al., JACS, 2003, 125, 8307, Wan et al. Nuc. Acid. Res., 2014, 42, 13456, and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

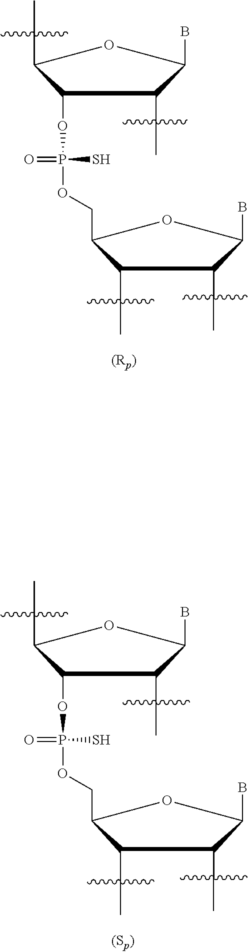

Unless otherwise indicated, chiral internucleoside linkages of modified oligonucleotides described herein can be stereorandom or in a particular stereochemical configuration.

In certain embodiments, modified oligonucleotides comprise an internucleoside motif of (5′ to 3′) sooosssssssssssssss. In certain embodiments, the particular stereochemical configuration of the modified oligonucleotides is (5′ to 3′) Sp-o-o-o-Sp-Sp-Sp-Rp-Sp-Sp-Rp-Sp-Sp-Sp-Sp-Sp-Sp-Sp-Sp or Sp-o-o-o-Sp-Sp-Sp-Rp-Sp-Sp-Sp-Sp-Sp-Sp-Sp-Sp-Sp-Sp-Sp; wherein each ‘Sp’ represents a phosphorothioate internucleoside linkage in the S configuration; Rp represents a phosphorothioate internucleoside linkage in the R configuration; and ‘o’ represents a phosphodiester internucleoside linkage.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (see e.g., Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, a modified internucleoside linkage is any of those described in WO 2021/030778, incorporated by reference herein.

### Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkages. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### Certain Sugar Motifs

In certain embodiments, oligonucleotides comprise one or more type of modified sugar and/or unmodified sugar moiety arranged along the oligonucleotide, or portion thereof, in a defined pattern or sugar motif. In certain instances, such sugar motifs include but are not limited to any of the sugar modifications discussed herein.

In certain embodiments, modified oligonucleotides have a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing of a gapmer comprises a modified sugar moiety. In certain embodiments, at least one, at least two, at least three, at least four, at least five, or at least six nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-deoxyribosyl sugar moiety. In certain embodiments, at least one nucleoside of the gap of a gapmer comprises a modified sugar moiety and each remaining nucleoside comprises a 2′-deoxyribosyl sugar moiety.

Herein, the lengths (number of nucleosides) of the three regions of a gapmer may be provided using the notation [# of nucleosides in the 5′-wing]-[# of nucleosides in the gap]-[# of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprise a 2′-deoxyribosyl sugar moiety. Thus, a 5-10-5 MOE gapmer consists of 5 linked 2′-MOE nucleosides in the 5′-wing, 10 linked 2′-deoxyribonucleosides in the gap, and 5 linked 2′-MOE nucleosides in the 3′-wing.

In certain embodiments, each nucleoside of a modified oligonucleotide, or portion thereof, comprises a 2′-substituted sugar moiety, a bicyclic sugar moiety, a sugar surrogate, or a 2′-deoxyribosyl sugar moiety. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, PNA, THP, and F-HNA.

In certain embodiments, modified oligonucleotides comprise at least 12, at least 13, at least 14, at least 15, at least 16, at least 17, at least 18, at least 19, or at least 20 nucleosides comprising a modified sugar moiety. In certain embodiments, the modified sugar moiety is selected independently from a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA.

In certain embodiments, each nucleoside of a modified oligonucleotide comprises a modified sugar moiety (“fully modified oligonucleotide”). In certain embodiments, each nucleoside of a fully modified oligonucleotide comprises a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, each nucleoside of a fully modified oligonucleotide comprises the same modified sugar moiety (“uniformly modified sugar motif”). In certain embodiments, the uniformly modified sugar motif is 7 to 20 nucleosides in length. In certain embodiments, each nucleoside of the uniformly modified sugar motif comprises a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA. In certain embodiments, modified oligonucleotides having at least one fully modified sugar motif may also comprise at least 1, at least 2, at least 3, or at least 4 2′-deoxyribonucleosides.

### Certain Nucleobase Motifs

In certain embodiments, oligonucleotides comprise modified and/or unmodified nucleobases arranged along the oligonucleotide, or portion thereof, in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of the nucleoside is a 2′-deoxyribosyl sugar moiety. In certain embodiments, the modified nucleobase is selected from: a 2-thiopyrimidine and a 5-propynepyrimidine.

### Certain Internucleoside Linkage Motifs

In certain embodiments, oligonucleotides comprise modified and/or unmodified internucleoside linkages arranged along the oligonucleotide, or portion thereof, in a defined pattern or motif. In certain embodiments, each internucleoside linking group is a phosphodiester internucleoside linkage. In certain embodiments, each internucleoside linking group of a modified oligonucleotide is a phosphorothioate internucleoside linkage. In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester internucleoside linkage is not a terminal internucleoside linkage, and the remaining internucleoside linkages are phosphorothioate internucleoside linkages. In certain such embodiments, all of the phosphorothioate internucleoside linkages are stereorandom. In certain embodiments, all of the phosphorothioate internucleoside linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs. In certain embodiments, one or more internucleoside linkage is a mesyl phosphoramidate internucleoside linkage. In certain embodiments, each internucleoside linkage is independently selected from a phosphodiester internucleoside linkage, a phosphorothioate internucleoside linkage, and a mesyl phosphoramidate internucleoside linkage. In certain embodiments, each internucleoside linkage is independently selected from a phosphorothioate internucleoside linkage and a mesyl phosphoramidate internucleoside linkage. In certain embodiments, one or more internucleoside linkage is a methoxypropylphosphonate internucleoside linkage. In certain embodiments, each internucleoside linkage is independently selected from a phosphodiester internucleoside linkage, a phosphorothioate internucleoside linkage, and a methoxypropylphosphonate internucleoside linkage. In certain embodiments, each internucleoside linkage is independently selected from a phosphorothioate internucleoside linkage and a methoxypropylphosphonate internucleoside linkage.

In certain embodiments, modified oligonucleotides comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 phosphodiester internucleoside linkages. In certain embodiments, modified oligonucleotides comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 phosphorothioate internucleoside linkages. In certain embodiments, modified oligonucleotides comprise at least 1, at least 2, at least 3, at least 4, or at least 5 phosphodiester internucleoside linkages and the remainder of the internucleoside linkages are phosphorothioate internucleoside linkages.

### Certain Lengths

It is possible to increase or decrease the length of an oligonucleotide without eliminating activity. For example, in Woolf et al., Proc. Natl. Acad. Sci. USA, 1992, 89, 7305-7309, 1992), a series of oligonucleotides 13-25 nucleobases in length were tested for their ability to induce cleavage of a target nucleic acid in an oocyte injection model. Oligonucleotides 25 nucleobases in length with 8 or 11 mismatch bases near the ends of the oligonucleotides were able to direct specific cleavage of the target nucleic acid, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, oligonucleotides consist of 16 linked nucleosides. In certain embodiments, oligonucleotides consist of 17 linked nucleosides. In certain embodiments, oligonucleotides consist of 18 linked nucleosides. In certain embodiments, oligonucleotides consist of 19 linked nucleosides. In certain embodiments, oligonucleotides consist of 20 linked nucleosides.

### Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a portion of an oligonucleotide has a nucleobase sequence that is complementary to a second oligonucleotide or an identified reference nucleic acid, such as a target nucleic acid. In certain embodiments, the nucleobase sequence of a portion or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

### Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge, and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

### Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, lipophilic groups, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial, or an antibiotic.

### Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain oligomeric compounds, a conjugate moiety is attached to an oligonucleotide via a more complex conjugate linker comprising one or more conjugate linker moieties, which are sub-units making up a conjugate linker. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxyribonucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate internucleoside linkage. In certain such embodiments, the cleavable moiety is 2′-deoxyadenosine.

### Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphanates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a portion complementary to a target nucleic acid and a second oligomeric compound having a portion complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

### Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce, modulate, or increase the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, provided herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid. In certain embodiments, hybridization of an antisense compound to a target nucleic acid results in exon inclusion. In certain embodiments, hybridization of an antisense compound to a target nucleic acid results in an increase in the amount or activity of a target nucleic acid. In certain embodiments, hybridization of an antisense compound complementary to a target nucleic acid results in alteration of splicing, leading to the inclusion of an exon in the mRNA.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

### Certain Target Nucleic Acids

In certain embodiments, oligomeric compounds comprise or consist of an oligonucleotide comprising a portion that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target nucleic acid is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron.

### Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a portion that is 100% or fully complementary to a target nucleic acid. In certain embodiments, the portion of full complementarity is 6, 7, 8, 9, 10, 11, 12, 13, 14, 15, 16, 17, 18, 19, or 20 nucleobases in length.

In certain embodiments, oligonucleotides comprise one or more mismatched nucleobases relative to the target nucleic acid. In certain embodiments, the mismatch is at position 1, 2, 3, 4, 5, 6, 7, 8, 9, 10, 11, 12, 13, 14, 15, 16, 17, 18, 19, or 20 from the 5′-end of the oligonucleotide.

### SMN2

In certain embodiments, oligomeric compounds comprise or consist of a modified oligonucleotide that is complementary to a target nucleic acid encoding SMN2, or a portion thereof. In certain embodiments, SMN2 has the sequence set forth in SEQ ID NO: 1 (GENBANK Accession No. NT_006713.14 truncated from nucleotides 19939708 to 19967777).

In certain embodiments, contacting a cell with the oligomeric compound complementary to SEQ ID NO: 1 modulates the splicing of SMN2 RNA in a cell. In certain embodiments, contacting a cell with the oligomeric compound complementary to SEQ ID NO: 1 increases the amount of SMN2 RNA including exon 7. In certain embodiments, contacting a cell with the oligomeric compound complementary to SEQ ID NO: 1 increases full-length SMN2 protein expression. In certain embodiments, the oligomeric compound consists of a modified oligonucleotide.

In certain embodiments, contacting a cell in a subject with an oligomeric compound complementary to SEQ ID NO: 1 ameliorates one or more symptom of a neurodegenerative disease. In certain embodiments, the neurodegenerative disease is SMA, including Type I SMA, Type II SMA, Type III SMA, and Type IV SMA. In certain embodiments, the symptom is any of reduced muscle strength; inability or reduced ability to sit upright, to stand, and/or walk; reduced neuromuscular activity; reduced electrical activity in one or more muscles; reduced respiration; inability or reduced ability to eat, drink, and/or breathe without assistance; loss of weight or reduced weight gain; and/or decreased survival.

In certain embodiments, an oligomeric compound complementary to SEQ ID NO: 1 is capable of increasing SMN2 RNA including exon 7 in vivo by at least 1 fold, 2 fold, or 3 fold when administered according to the standard in vivo assay. In certain embodiments, an oligomeric compound complementary to SEQ ID NO: 1 is capable of increasing full-length SMN2 protein in vivo by at least 1 fold, 2 fold, or 3 fold when administered according to the standard in vivo assay.

### Certain Target Nucleic Acids in Certain Tissues

In certain embodiments, oligomeric compounds comprise or consist of an oligonucleotide comprising a portion that is complementary to a target nucleic acid, wherein the target nucleic acid is expressed in a pharmacologically relevant tissue. In certain embodiments, the pharmacologically relevant tissues are the cells and tissues that comprise the central nervous system (CNS). Such tissues include brain tissues, such as, spinal cord, cortex, and coronal brain tissue.

### Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid (“artificial CSF” or “aCSF”). In certain embodiments, the artificial cerebrospinal fluid is pharmaceutical grade. In certain embodiments, a pharmaceutical composition comprises a modified oligonucleotide and artificial cerebrospinal fluid. In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents comprising an oligomeric compound provided herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphate linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a salt thereof” expressly includes all such forms that may be fully or partially protonated/de-protonated/in association with a cation. In certain instances, one or more specific cation is identified.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid. For example, where a modified oligonucleotide or an oligomeric compound is in solution comprising sodium (e.g., saline), the modified oligonucleotide or oligomeric compound may be partially or fully de-protonated and in association with Na+ ions. However, the mass of the protons are nevertheless counted toward the weight of the dose, and the mass of the Na+ ions are not counted toward the weight of the dose. Thus, for example, a dose, or dosage unit, of 10 mg of Compound No. 1263789, Compound No. 1287717, Compound No. 1287745, and Compound No. 1358996 equals the number of fully protonated molecules that weighs 10 mg. This would be equivalent to 10.53 mg of solvent-free, sodium acetate-free, anhydrous sodiated Compound No. 1263789, 10.53 mg of solvent-free, sodium acetate-free, anhydrous sodiated Compound No. 1287717, 10.52 mg of solvent-free, sodium acetate-free, anhydrous sodiated Compound No. 1287745, and 10.51 mg of solvent-free, sodium acetate-free, anhydrous sodiated Compound No. 1358996. When an oligomeric compound comprises a conjugate group, the mass of the conjugate group is included in calculating the dose of such oligomeric compound. If the conjugate group also has an acid, the conjugate group is likewise assumed to be fully protonated for the purpose of calculating dose.

### Certain Compositions

### Compound No: 1263789

In certain embodiments, Compound No. 1263789 is characterized as a modified oligonucleotide having a sequence of (from 5′ to 3′) CACTTTCATAATGCTGGC (SEQ ID NO: 21), wherein each nucleoside comprises a 2′-MOE sugar moiety, wherein the internucleoside linkages between nucleosides 2 to 3 and 4 to 5 are phosphodiester internucleoside linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 16 to 17, and 17 to 18 are phosphorothioate internucleoside linkages, and wherein each cytosine is a 5-methyl cytosine.

In certain embodiments, Compound No. 1263789 is represented by the following chemical notation (5′ to 3′): ^{m}Cₑₛ Aₑₒ ^{m}Cₑₛ Tₑₒ Tₑₛ Tₑₛ ^{m}Cₑₛ Aₑₛ Tₑₛ Aₑₛ Aₑₛ Tₑₛ Gₑₛ ^{m}Cₑₛ Tₑₛ Gₑₛ Gₑₛ ^{m}Cₑ (SEQ ID NO: 21),

wherein,
A=an adenine nucleobase,
^{m}C=a 5-methyl cytosine nucleobase,
G=a guanine nucleobase,
T=a thymine nucleobase,
e=a 2′-MOE sugar moiety,
s=a phosphorothioate internucleoside linkage, and
o=a phosphodiester internucleoside linkage.

In certain embodiments, Compound No. 1263789 is represented by the following chemical structure:

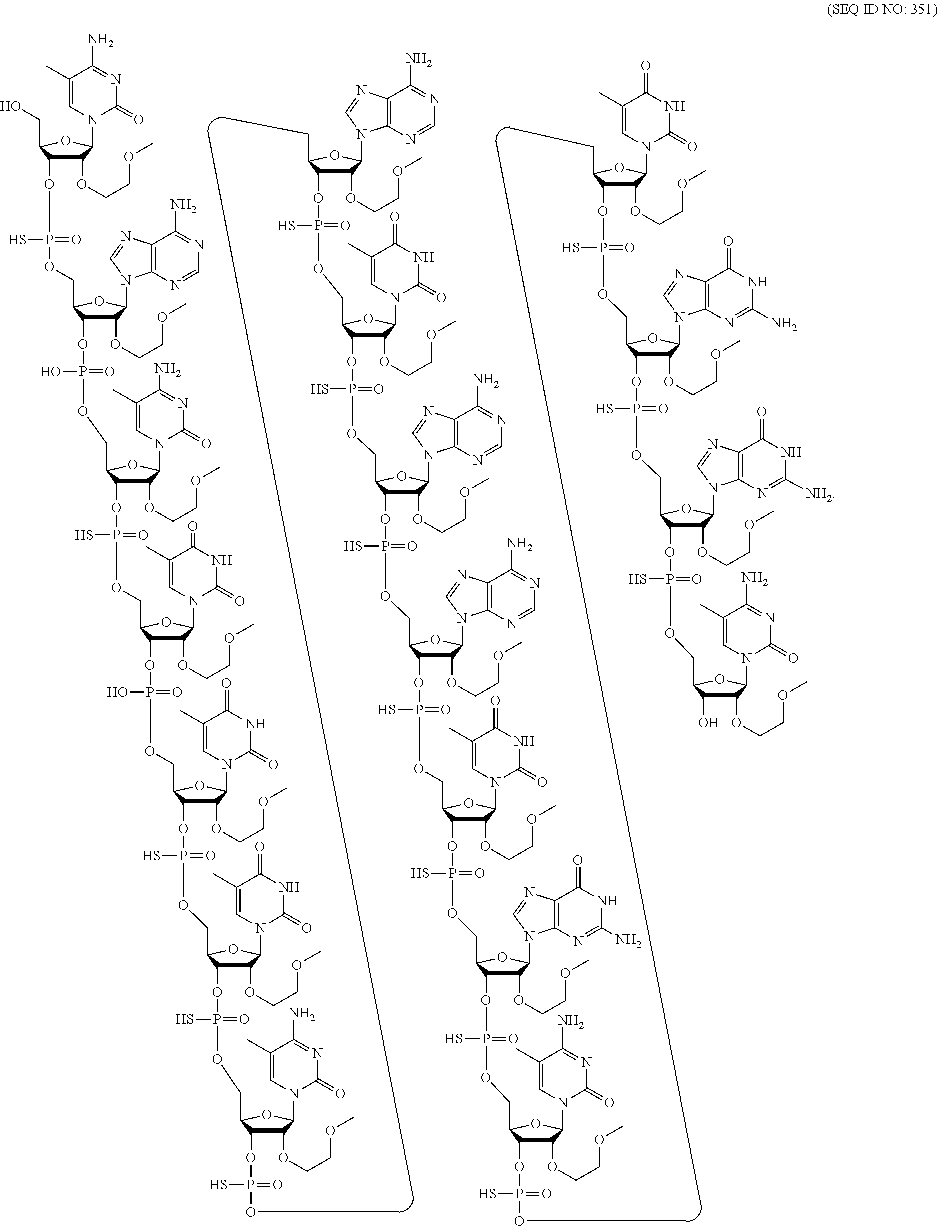

### Structure 1. Compound No. 1263789

In certain embodiments, the sodium salt of Compound No. 1263789 is represented by the following chemical structure:

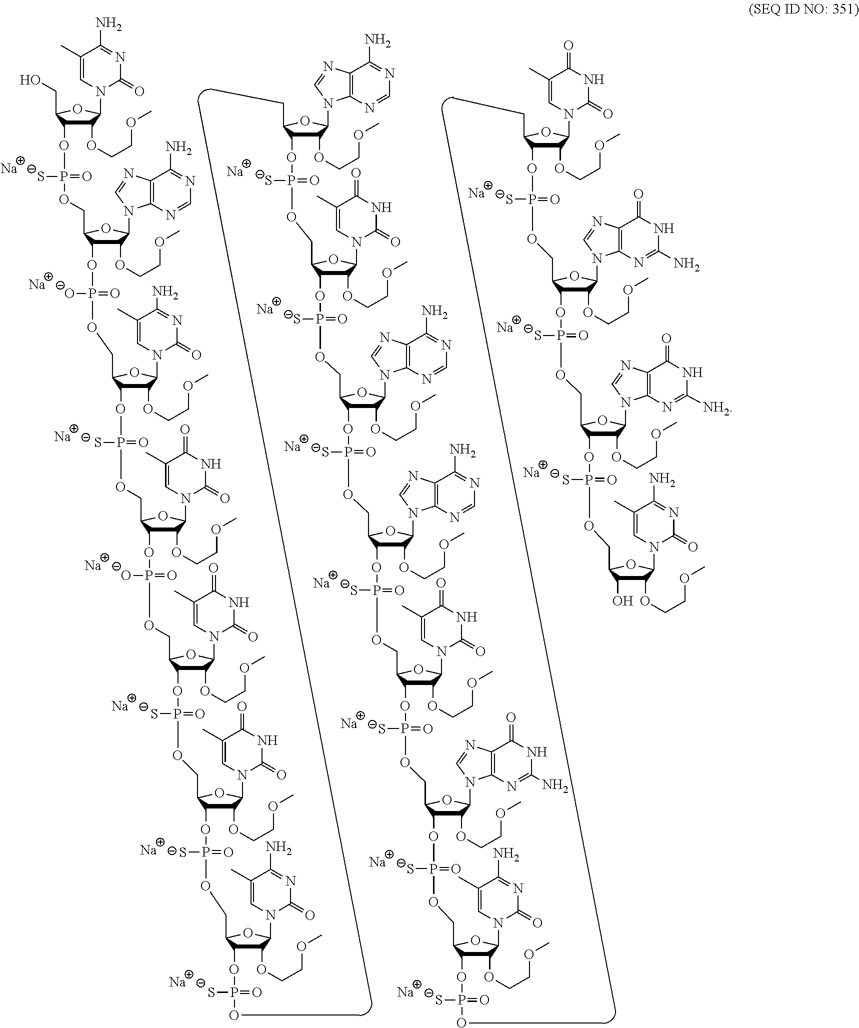

Structure 2. The sodium salt of Compound No. 1263789

### Compound No: 1287717

In certain embodiments, Compound No. 1287717 is characterized as a modified oligonucleotide having a sequence of (from 5′ to 3′) TTCACTTTCATAATGCTGGC (SEQ ID NO: 22), wherein each nucleoside comprises a 2′-MOE sugar moiety, wherein the internucleoside linkages between nucleosides 1 to 2 and 19 to 20 are phosphodiester internucleoside linkages and the internucleoside linkages between nucleosides 2 to 3, 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 are phosphorothioate internucleoside linkages, and wherein each cytosine is a 5-methyl cytosine.

In certain embodiments, Compound No. 1287717 is represented by the following chemical notation (5′ to 3′): Tₑₒ Tₑₛ ^{m}Cₑₛ Aₑₛ ^{m}Cₑₛ Tₑₛ Tₑₛ Tₑₛ ^{m}Cₑₛ Aₑₛ Tₑₛ Aₑₛ Aₑₛ Tₑₛ Gₑₛ ^{m}Cₑₛ Tₑₛ Gₑₛ Gₑₒ ^{m}Cₑ (SEQ ID NO: 22)

wherein,
A=an adenine nucleobase,
^{m}C=a 5-methyl cytosine nucleobase,
G=a guanine nucleobase,
T=a thymine nucleobase,
e=a 2′-MOE sugar moiety,
s=a phosphorothioate internucleoside linkage, and
o=a phosphodiester internucleoside linkage.

In certain embodiments, Compound No. 1287717 is represented by the following chemical structure:

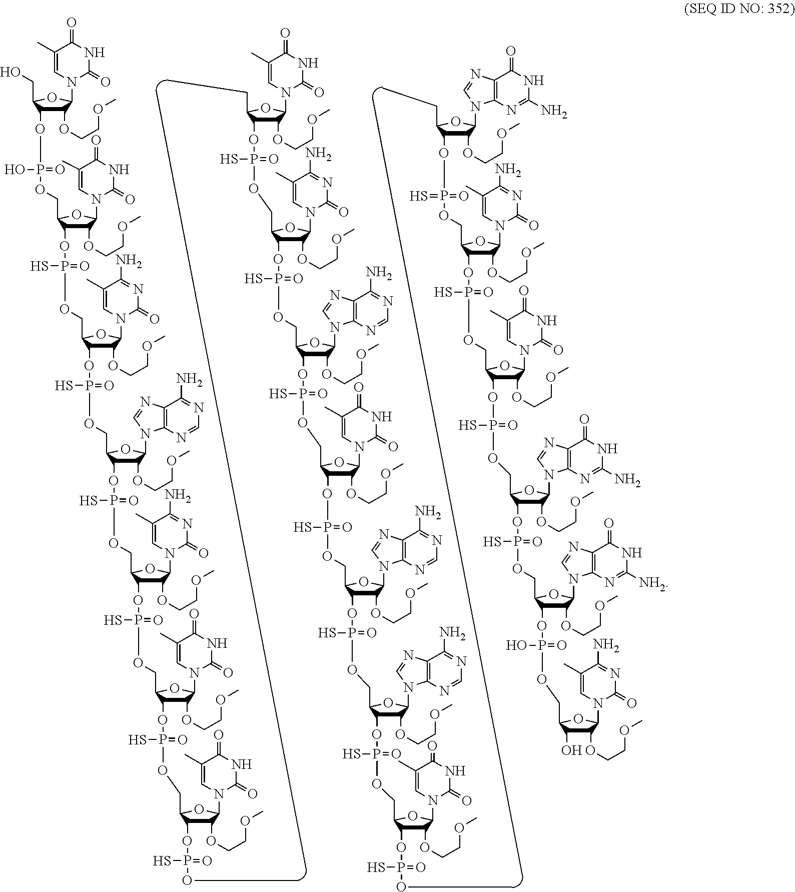

### Structure 3. Compound No. 1287717

In certain embodiments, the sodium salt of Compound No. 1287717 is represented by the following chemical structure:

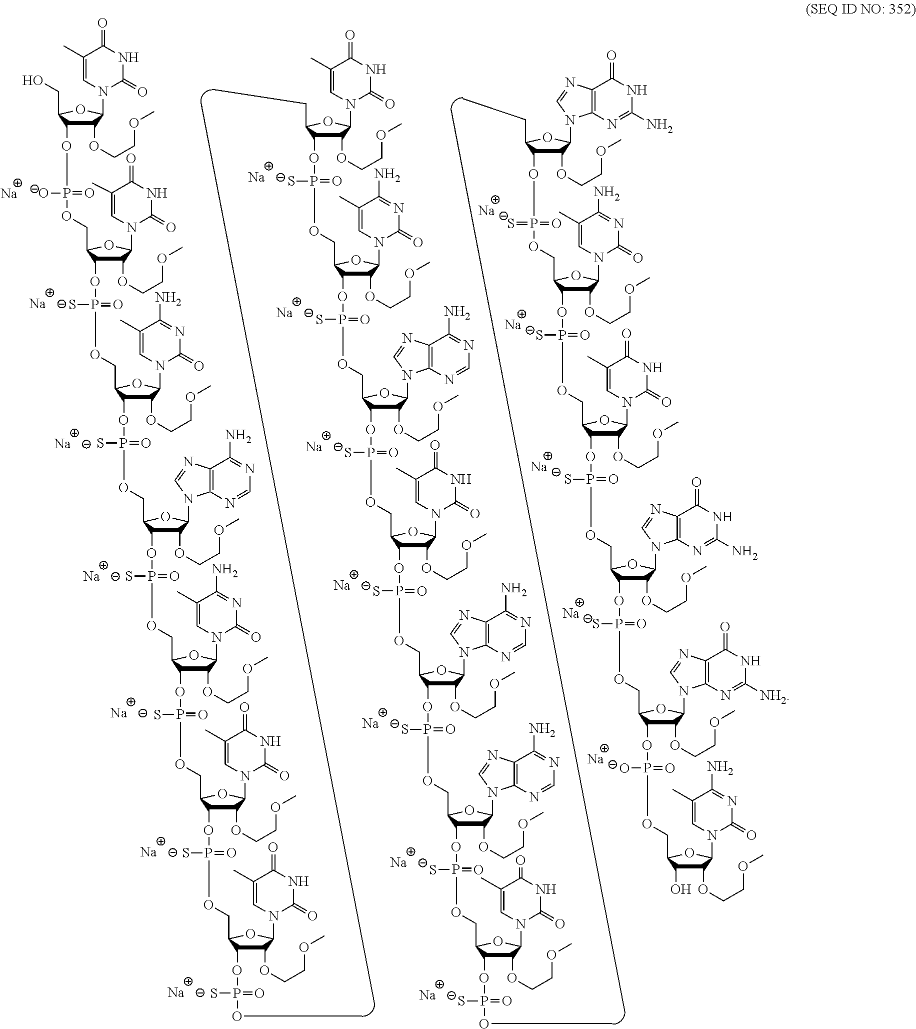

### Structure 4. The Sodium Salt of Compound No. 1287717

### Compound No: 1287745

In certain embodiments, Compound No. 1287745 is characterized as a modified oligonucleotide having a sequence of (from 5′ to 3′) TTCACTTTCATAATGCTGGC (SEQ ID NO: 51), wherein each of nucleosides 1 and 20 comprises a 2′-MOE sugar moiety, each of nucleosides 2-19 comprises a 2′-NMA sugar moiety, wherein the internucleoside linkages between nucleosides 1 to 2 and 19 to 20 are phosphodiester internucleoside linkages and the internucleoside linkages between nucleosides 2 to 3, 3 to 4, 4 to 5, 5 to 6, 6 to 7, 7 to 8, 8 to 9, 9 to 10, 10 to 11, 11 to 12, 12 to 13, 13 to 14, 14 to 15, 15 to 16, 16 to 17, 17 to 18, and 18 to 19 are phosphorothioate internucleoside linkages, and wherein each cytosine is a 5-methyl cytosine.

In certain embodiments, Compound No. 1287745 is represented by the following chemical notation (5′ to 3′): Tₑₒ Tₙₛ ^{m}Cₙₛ ^{m}Cₙₛ Tₙₛ ^{m}Cₙₛ Aₙₛ Tₙₛ Aₙₛ Aₙₛ Tₙₛ Gₙₛ ^{m}Cₙₛ Tₙₛ Gₙₛ Gₙₒ ^{m}Cₑ (SEQ ID NO: 51) wherein,
A=an adenine nucleobase,
^{m}C=a 5-methyl cytosine nucleobase,
G=a guanine nucleobase,
T=a thymine nucleobase,
e=a 2′-MOE sugar moiety,
n=a 2′-NMA sugar moiety,
s=a phosphorothioate internucleoside linkage, and
o=a phosphodiester internucleoside linkage.

In certain embodiments, Compound No. 1287745 is represented by the following chemical structure:

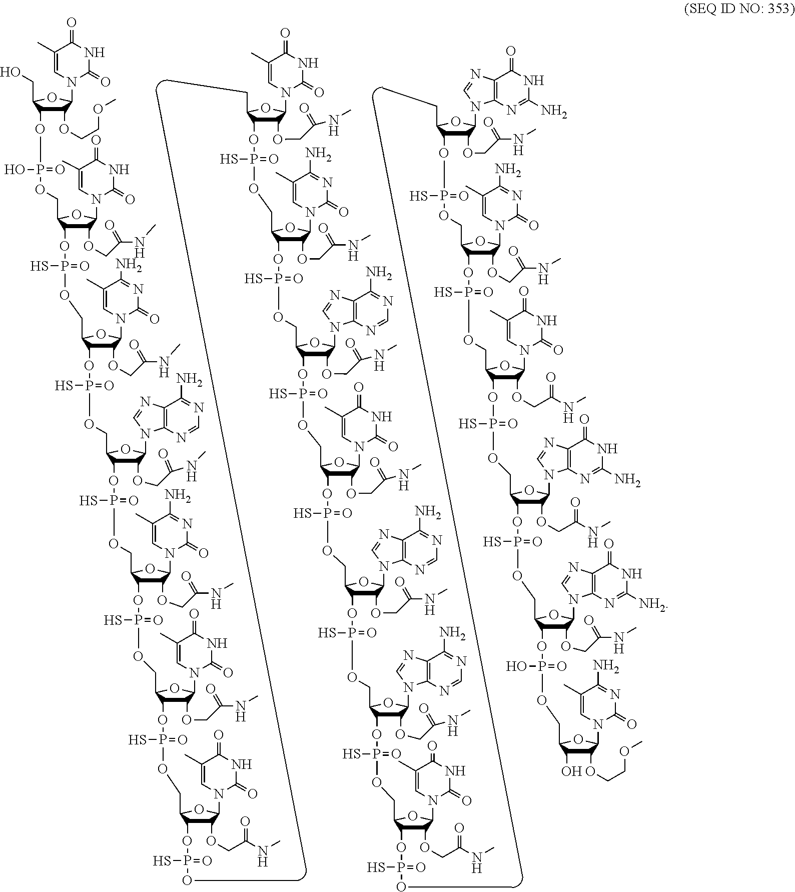

### Structure 5. Compound No. 1287745

In certain embodiments, the sodium salt of Compound No. 1287745 is represented by the following chemical structure:

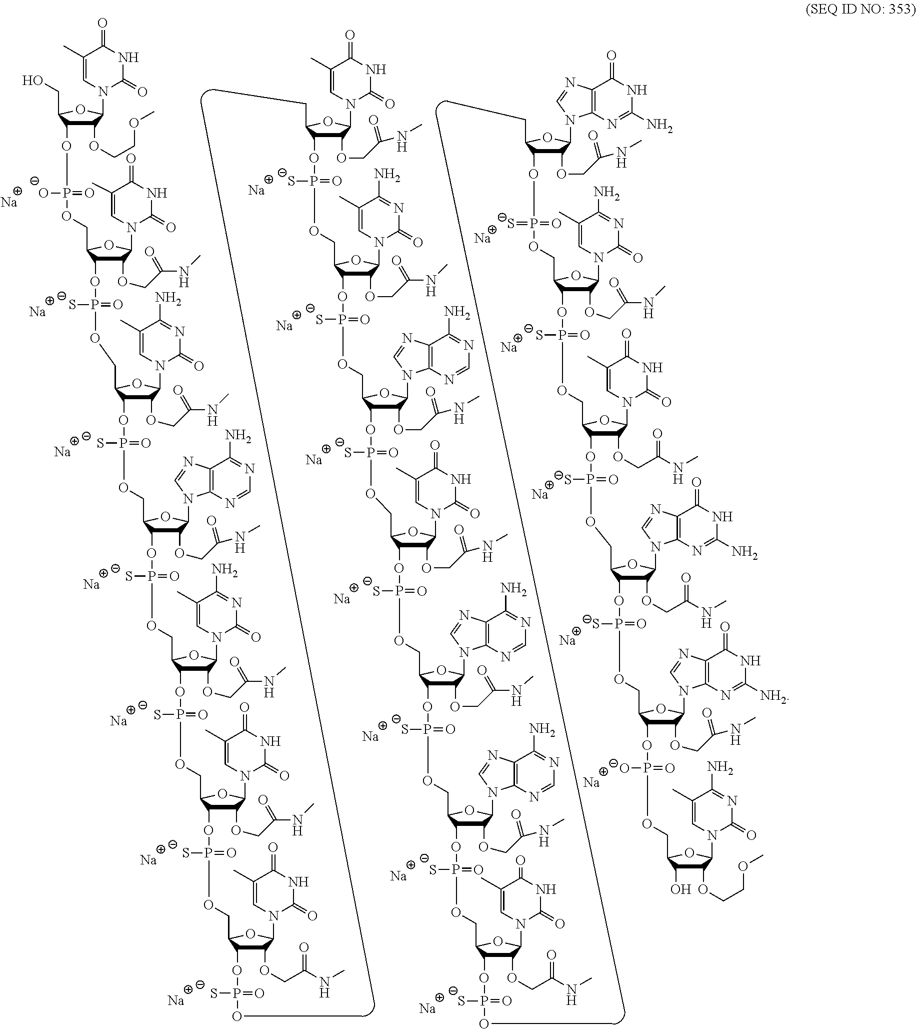

### Structure 6. The Sodium Salt of Compound No. 1287745

### Compound No: 1358996

In certain embodiments, Compound No. 1358996 is characterized as modified oligonucleotide having a sequence of (from 5′ to 3′) CACTTTCATAATGCTGGC (SEQ ID NO: 52), wherein each nucleoside comprises a 2′-NMA sugar moiety, wherein the internucleoside linkages between nucleosides 2 to 3 and 4 to 5 are phosphodiester internucleoside linkages and the internucleoside linkages between nucleosides 1 to 2, 3 to 4, 5 to 6, 6 to 7, 7 to 8, 8 to 9, 9 to 10, 10 to 11, 11 to 12, 12 to 13, 13 to 14, 14 to 15, 15 to 16, 16 to 17, and 17 to 18 are phosphorothioate internucleoside linkages, and wherein each cytosine is a 5-methyl cytosine.

In certain embodiments, Compound No. 1358996 is represented by the following chemical notation (5′ to 3′): ^{m}Cₙₛ ^{m}Cₙₛ Tₙₒ Tₙₛ Tₙₛ ^{m}Cₙₛ Aₙₛ Tₙₛ Aₙₛ Aₙₛ Tₙₛ Gₙₛ ^{m}Cₙₛ Tₙₛ Gₙₛ Gₙₛ ^{m}Cₙ (SEQ ID NO: 52)

wherein,
A=an adenine nucleobase,
^{m}C=a 5-methyl cytosine nucleobase,
G=a guanine nucleobase,
T=a thymine nucleobase,
n=a 2′-NMA sugar moiety,
s=a phosphorothioate internucleoside linkage, and
o=a phosphodiester internucleoside linkage.

In certain embodiments, Compound No. 1358996 is represented by the following chemical structure:

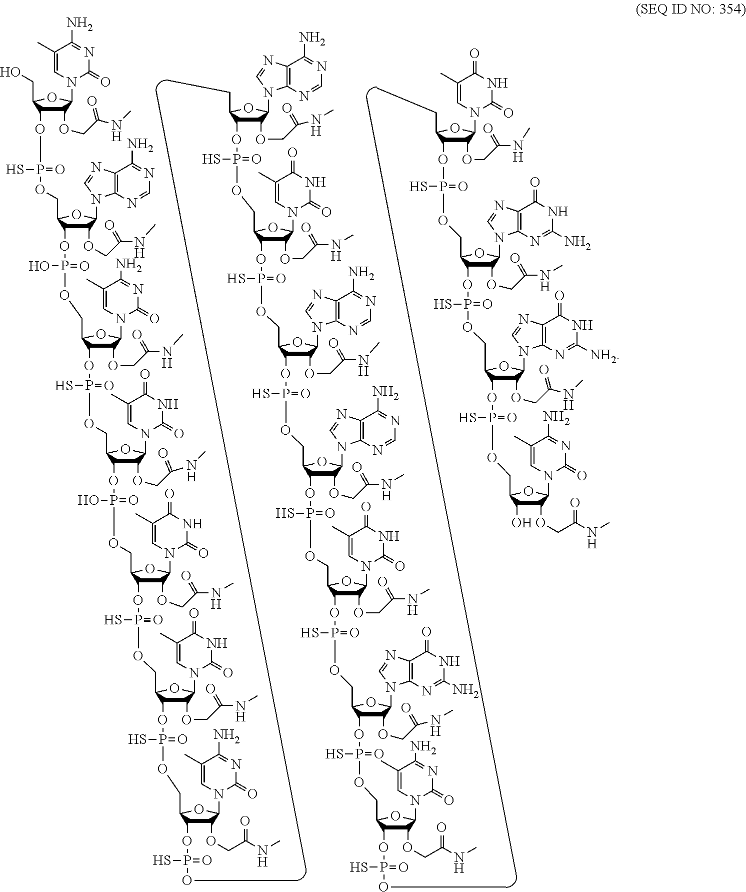

### Structure 7. Compound No. 1358996

In certain embodiments, the sodium salt of Compound No. 1358996 is represented by the following chemical structure:

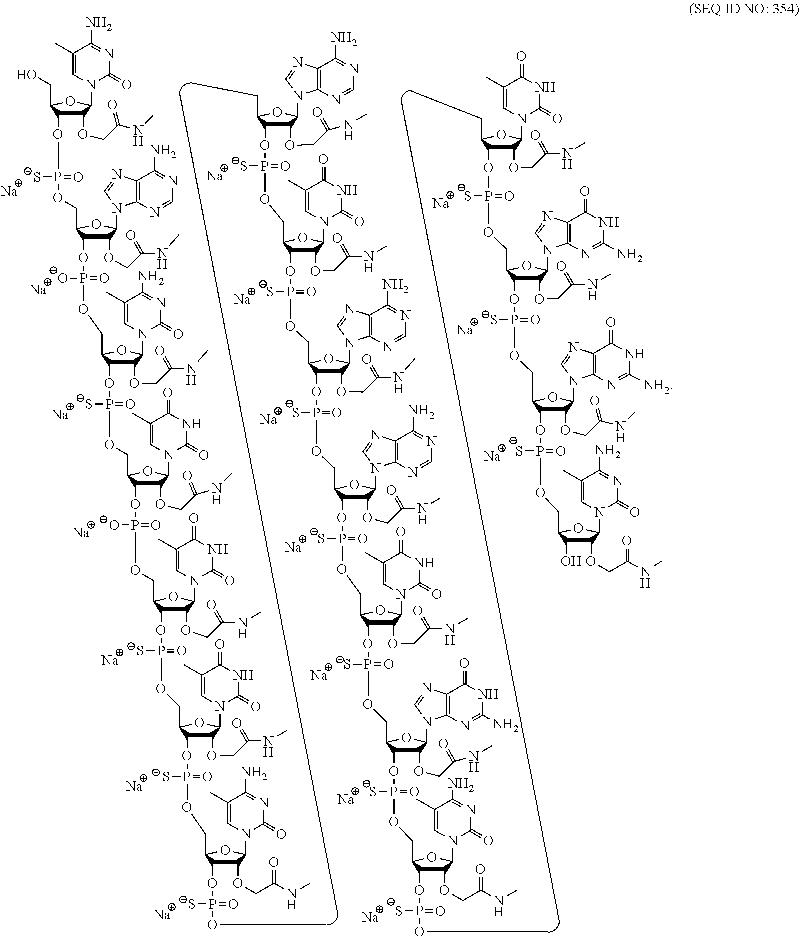

### Structure 8. The Sodium Salt of Compound No. 1358996

### Certain Comparator Compositions

In certain embodiments, Spinraza® (generic name nusinersen; Compound No. 396443), approved for treatment of SMA, is a comparator compound (See, e.g., Chiroboga, et al., Neurology, 86(10): 890-897, 2016; Finkel, et al., Lancet, 338(10063): 3017-3026, 2016; Finkel, et al., N. Engl. J. Med., 377(18):1723-1732 2017; Mercuri, et al., N. Engl. J. Med., 378(7):625-635, 2018; Montes, et al., Muscle Nerve. 60(4): 409-414, 2019; Darras, et al., Neurology, 92(21):e2492-e2506, 2019). Spinraza® was previously described in WO2010120820, incorporated herein by reference, and has a sequence (from 5′ to 3′) of TCACTTTCATAATGCTGG (SEQ ID NO: 355), wherein each nucleoside comprises a 2′-MOE sugar moiety, each internucleoside linkage is a phosphorothioate internucleoside linkage, and each cytosine is a 5-methyl cytosine.

In certain embodiments, although not approved for human therapy, other previously described compounds, including Compound Nos. 387954, 396442, 443305, and 819735, are comparator compounds.

Compound No. 387954 was previously described in WO 2014/179620, incorporated herein by reference. Compound No. 387954 has a sequence (from 5′ to 3′) of ATTCACTTTCATAATGCTGG (SEQ ID NO: 20), wherein each nucleoside comprises a 2′-MOE sugar moiety, each internucleoside linkage is a phosphorothioate internucleoside linkage, and each cytosine is a 5-methyl cytosine.

Compound No. 396442 was previously described in WO 2010/120820, incorporated herein by reference. Compound No. 396442 has a sequence (from 5′ to 3′) of CACTTTCATAATGCTGGC (SEQ ID NO: 53), wherein each nucleoside comprises a 2′-MOE sugar moiety, each internucleoside linkage is a phosphorothioate internucleoside linkage, and each cytosine is a 5-methyl cytosine.

Compound No. 443305 was previously described in WO 2018/014041, incorporated herein by reference. Compound No. 443305 has a sequence (from 5′ to 3′) of TCACTTTCATAATGCTGG (SEQ ID NO: 54), wherein each nucleoside comprises a 2′-NMA sugar moiety, each internucleoside linkage is a phosphorothioate internucleoside linkage, and each cytosine is a 5-methyl cytosine.

Compound No. 819735 was previously described in WO 2018/014041, incorporated herein by reference. Compound No. 819735 has a sequence (from 5′ to 3′) of CACTTTCATAATGCTGGC (SEQ ID NO: 55), wherein each nucleoside comprises a 2′-NMA sugar moiety, each internucleoside linkage is a phosphorothioate internucleoside linkage, and each cytosine is a 5-methyl cytosine.

TABLE 1
Certain Comparator Compositions

| | | | | SEQ | |
|---|---|---|---|---|---|
| Compound | Nucleobase Sequence | | Internucleoside | ID | |
| Number | (5′ to 3′) | Sugar Motif | Linkage Motif | NO: | Reference Number |
| 396443 | TCACTTTCATAATGCTGG | Full 2′- MOE | Full PS | 23 | WO 2010/120820 |
| 387954 | ATTCACTTTCATAATGCTGG | Full 2′- MOE | Full PS | 20 | WO 2014/179620 |
| 396442 | CACTTTCATAATGCTGGC | Full 2′- MOE | Full PS | 21 | WO 2010/120820 |
| 443305 | TCACTTTCATAATGCTGG | Full 2′- NMA | Full PS | 23 | WO 2018/014041 |
| 819735 | CACTTTCATAATGCTGGC | Full 2′- NMA | Full PS | 21 | WO 2018/014041 |

In certain embodiments, compounds described herein are superior relative to compounds described in WO 2007/002390, WO2010/120820, WO 2015/161170, and WO 2018/014041, because they demonstrate one or more improved properties, such as, potency, efficacy, and tolerability.

For example, Compound No. 1263789, Compound No. 1287745, and Compound No. 1358996 each demonstrated improved potency in vivo as compared to Compound No. 396443. As shown in Example 5, Compound No. 1263789, Compound No. 1287745, and Compound No. 1358996 achieved an ED₅₀ in spinal cord of 13.3, 8.8, and 7.4, respectively. In comparison, Compound No. 396443 achieved an ED₅₀ in spinal cord of 22.0. Therefore, each of Compound No. 1263789, Compound No. 1287745, and Compound No. 1358996 are more potent than Compound No. 396443 in this assay.

For example, Compound No. 1263789, Compound No. 1287717, Compound No. 1287745, and Compound No. 1358996 each demonstrated improved 3 hour FOB scores as compared to Compound No. 396443, Compound No. 387954, and Compound No. 443305. As shown in Example 6, at 700 μg, Compound No. 1263789, Compound No. 1287717, Compound No. 1287745, and Compound No. 1358996 achieved 3 hour FOB scores of 0, 3.25, 1, and 0, respectively. In comparison, at half the dose (350 μg) Compound No. 396443 achieved a 3 hour FOB score of 4.0; and at the same dose (700 μg) Compound No. 387954 and Compound No. 443305 achieved a 3 hour FOB score of 4.0 and 4.75, respectively. Therefore, each of Compound No. 1263789, Compound No. 1287717, Compound No. 1287745, and Compound No. 1358996 are more tolerable than Compound No. 396443, Compound No. 387954, and Compound No. 443305 in this assay.

For example, Compound No. 1263789, Compound No. 1287717, Compound No. 1287745, and Compound No. 1358996 each demonstrated improved long-term tolerability as compared to Compound No. 396442 and Compound No. 819735. As shown in Example 7, Compound No. 1263789, Compound No. 1287717, Compound No. 1287745, and Compound No. 1358996 demonstrated no adverse events, no Purkinje cell loss, and cortex GFAP mRNA less than 2-fold of control. In comparison, 396442 and 819735 each demonstrated adverse events, Purkinje cell loss, and cortex GFAP mRNA greater than 2-fold of control in certain treated animals. Therefore, each of Compound No. 1263789, Compound No. 1287717, Compound No. 1287745, and Compound No. 1358996 are more tolerable than Compound No. 396442 and Compound No. 819735 in this assay.

### Nonlimiting Disclosure and Incorporation by Reference

Each of the literature and patent publications listed herein is incorporated by reference in its entirety. While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar moiety (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, all cis- and trans-isomers and tautomeric forms of the compounds herein are also included unless otherwise indicated. Oligomeric compounds described herein include chirally pure or enriched mixtures as well as racemic mixtures. For example, oligomeric compounds having a plurality of phosphorothioate internucleoside linkages include such compounds in which chirality of the phosphorothioate internucleoside linkages is controlled or is random. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments.

### Example 1: Design of Modified Oligonucleotides Complementary to a Human SMN2 Nucleic Acid

Modified oligonucleotides complementary to a human SMN2 nucleic acid were designed and synthesized as indicated in the tables below.

The modified oligonucleotides in the tables below are 16, 17, 18, 19, or 20 nucleosides in length, as specified. The modified oligonucleotides comprise 2′-MOE sugar moieties, 2′-NMA sugar moieties, cEt sugar moieties, 2′-OMe sugar moieties, and/or 2′-β-D-deoxyribosyl sugar moieties, as specified. Each internucleoside linkage throughout the modified oligonucleotides is either a phosphorothioate internucleoside linkage or a phosphodiester internucleoside linkage, as specified. Cytosines are either non-methylated cytosines or 5-methyl cytosines, as specified.

Each modified oligonucleotide listed in the tables below is 100% complementary to SEQ ID NO: 1 (GENBANK Accession No. NT_006713.14 truncated from nucleotides 19939708 to 19967777), unless specifically stated otherwise. Non-complementary nucleobases are specified in the Nucleobase Sequence column in underlined, bold, italicized font. Each modified oligonucleotide listed in the tables below targets an active site on the SMN2 transcript for inclusion of exon 7. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

### Table 2

The modified oligonucleotides in Table 2 below are 16, 17, 18, 19 or 20 nucleosides in length. Each nucleoside comprises a 2′-MOE sugar moiety. The sugar motif for each modified oligonucleotide is provided in the Sugar Motif column, wherein each ‘e’ represents a 2′-MOE sugar moiety. Each internucleoside linkage is either a phosphorothioate internucleoside linkage or a phosphodiester internucleoside linkage. The internucleoside linkage motif for each modified oligonucleotide is provided in the Internucleoside Linkage Motif column, wherein each ‘s’ represents a phosphorothioate internucleoside linkage, and each ‘o’ represents a phosphodiester internucleoside linkage. Each cytosine is a 5-methyl cytosine.

Each modified oligonucleotide listed in Table 2 below is 100% complementary to SEQ ID NO: 1 (GENBANK Accession No. NT_006713.14 truncated from nucleotides 19939708 to 19967777), unless specifically stated otherwise. Non-complementary nucleobases are specified in the Nucleobase Sequence column in underlined, bold, italicized font. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

TABLE 2
2′-MOE modified oligonucleotides with PS or mixed PS/PO internucleoside linkages

| | | | | SEQ | SEQ | |
|---|---|---|---|---|---|---|
| | | | | ID | ID | |
| | | | | No: 1 | No: 1 | SEQ |
| Compound | Nucleobase Sequence | Sugar Motif | Internucleoside | Start | Stop | ID |
| Number | (5′ to 3′) | (5′ to 3′) | Linkage Motif (5′ to 3′) | Site | Site | No. |
| 1287063 | ACTTTCATAATGCTGGCAG | eeeeeeeeeeeeeeeeeee | ssssssssssssssssss | 27059 | 27077 | 24 |
| 1287048 | CACTTTCATAATGCTGGCAG | eeeeeeeeeeeeeeeeeeee | sssssssssssssssssss | 27059 | 27078 | 25 |
| 1287064 | CACTTTCATAATGCTGGCA | eeeeeeeeeeeeeeeeeee | ssssssssssssssssss | 27060 | 27078 | 26 |
| 1287049 | TCACTTTCATAATGCTGGCA | eeeeeeeeeeeeeeeeeeee | sssssssssssssssssss | 27060 | 27079 | 27 |
| 1210340 | CTTTCATAATGCTGGC | eeeeeeeeeeeeeeee | sssssssssssssss | 27061 | 27076 | 28 |
| 1212868 | CTTTCATAATGCTGGC | eeeeeeeeeeeeeeee | sssssssssooooss | 27061 | 27076 | 28 |
| 1212867 | CTTTCATAATGCTGGC | eeeeeeeeeeeeeeee | ssssssssoooosss | 27061 | 27076 | 28 |
| 1212863 | CTTTCATAATGCTGGC | eeeeeeeeeeeeeeee | ssssssooossssss | 27061 | 27076 | 28 |
| 1212866 | CTTTCATAATGCTGGC | eeeeeeeeeeeeeeee | ssssssoooosssss | 27061 | 27076 | 28 |
| 1212861 | CTTTCATAATGCTGGC | eeeeeeeeeeeeeeee | sssssooooosssss | 27061 | 27076 | 28 |
| 1212860 | CTTTCATAATGCTGGC | eeeeeeeeeeeeeeee | sssssoooooossss | 27061 | 27076 | 28 |
| 1212865 | CTTTCATAATGCTGGC | eeeeeeeeeeeeeeee | ssssoooosssssss | 27061 | 27076 | 28 |
| 1212859 | CTTTCATAATGCTGGC | eeeeeeeeeeeeeeee | ssssooooooossss | 27061 | 27076 | 28 |
| 1212851 | CTTTCATAATGCTGGC | eeeeeeeeeeeeeeee | sssosssosssosss | 27061 | 27076 | 28 |
| 1212850 | CTTTCATAATGCTGGC | eeeeeeeeeeeeeeee | ssosssssssssoss | 27061 | 27076 | 28 |
| 1212852 | CTTTCATAATGCTGGC | eeeeeeeeeeeeeeee | ssossossossosss | 27061 | 27076 | 28 |
| 1212853 | CTTTCATAATGCTGGC | eeeeeeeeeeeeeeee | ssossossosososs | 27061 | 27076 | 28 |
| 1212854 | CTTTCATAATGCTGGC | eeeeeeeeeeeeeeee | ssososososososs | 27061 | 27076 | 28 |
| 1212864 | CTTTCATAATGCTGGC | eeeeeeeeeeeeeeee | ssoooosssssssss | 27061 | 27076 | 28 |
| 1212855 | CTTTCATAATGCTGGC | eeeeeeeeeeeeeeee | soossssssssooss | 27061 | 27076 | 28 |
| 1212856 | CTTTCATAATGCTGGC | eeeeeeeeeeeeeeee | sooosssssssooss | 27061 | 27076 | 28 |
| 1212857 | CTTTCATAATGCTGGC | eeeeeeeeeeeeeeee | sooossssssoooss | 27061 | 27076 | 28 |
| 1212858 | CTTTCATAATGCTGGC | eeeeeeeeeeeeeeee | soooosssssoooss | 27061 | 27076 | 28 |
| 1210339 | ACTTTCATAATGCTGGC | eeeeeeeeeeeeeeeee | ssssssssssssssss | 27061 | 27077 | 29 |
| 1212849 | ACTTTCATAATGCTGGC | eeeeeeeeeeeeeeeee | ssssssssssooooss | 27061 | 27077 | 29 |
| 1212848 | ACTTTCATAATGCTGGC | eeeeeeeeeeeeeeeee | ssssssssoooossss | 27061 | 27077 | 29 |
| 1212845 | ACTTTCATAATGCTGGC | eeeeeeeeeeeeeeeee | sssssssooossssss | 27061 | 27077 | 29 |
| 1212844 | ACTTTCATAATGCTGGC | eeeeeeeeeeeeeeeee | ssssssoooossssss | 27061 | 27077 | 29 |
| 1212843 | ACTTTCATAATGCTGGC | eeeeeeeeeeeeeeeee | ssssssooooosssss | 27061 | 27077 | 29 |
| 1212842 | ACTTTCATAATGCTGGC | eeeeeeeeeeeeeeeee | sssssoooooosssss | 27061 | 27077 | 29 |
| 1212841 | ACTTTCATAATGCTGGC | eeeeeeeeeeeeeeeee | sssssooooooossss | 27061 | 27077 | 29 |
| 1212847 | ACTTTCATAATGCTGGC | eeeeeeeeeeeeeeeee | ssssoooossssssss | 27061 | 27077 | 29 |
| 1212832 | ACTTTCATAATGCTGGC | eeeeeeeeeeeeeeeee | ssossssssssssoss | 27061 | 27077 | 29 |
| 1212833 | ACTTTCATAATGCTGGC | eeeeeeeeeeeeeeeee | ssosssssossssoss | 27061 | 27077 | 29 |
| 1212834 | ACTTTCATAATGCTGGC | eeeeeeeeeeeeeeeee | ssosssosssossoss | 27061 | 27077 | 29 |
| 1212835 | ACTTTCATAATGCTGGC | eeeeeeeeeeeeeeeee | ssossossossososs | 27061 | 27077 | 29 |
| 1212836 | ACTTTCATAATGCTGGC | eeeeeeeeeeeeeeeee | ssososososososss | 27061 | 27077 | 29 |
| 1212846 | ACTTTCATAATGCTGGC | eeeeeeeeeeeeeeeee | ssoooossssssssss | 27061 | 27077 | 29 |
| 1212837 | ACTTTCATAATGCTGGC | eeeeeeeeeeeeeeeee | soosssssssssooss | 27061 | 27077 | 29 |
| 1212838 | ACTTTCATAATGCTGGC | eeeeeeeeeeeeeeeee | sooossssssssooss | 27061 | 27077 | 29 |
| 1212839 | ACTTTCATAATGCTGGC | eeeeeeeeeeeeeeeee | sooosssssssoooss | 27061 | 27077 | 29 |
| 1212840 | ACTTTCATAATGCTGGC | eeeeeeeeeeeeeeeee | soooossssssoooss | 27061 | 27077 | 29 |
| 1263814 | CACTTTCATAATGCTGGC | eeeeeeeeeeeeeeeeee | ssossssssssssssss | 27061 | 27078 | 21 |
| 1263816 | CACTTTCATAATGCTGGC | eeeeeeeeeeeeeeeeee | ssssossssssssssss | 27061 | 27078 | 21 |
| 1263818 | CACTTTCATAATGCTGGC | eeeeeeeeeeeeeeeeee | ssssssossssssssss | 27061 | 27078 | 21 |
| 1263820 | CACTTTCATAATGCTGGC | eeeeeeeeeeeeeeeeee | ssssssssossssssss | 27061 | 27078 | 21 |
| 1263822 | CACTTTCATAATGCTGGC | eeeeeeeeeeeeeeeeee | ssssssssssossssss | 27061 | 27078 | 21 |
| 1263824 | CACTTTCATAATGCTGGC | eeeeeeeeeeeeeeeeee | ssssssssssssossss | 27061 | 27078 | 21 |
| 1263826 | CACTTTCATAATGCTGGC | eeeeeeeeeeeeeeeeee | ssssssssssssssoss | 27061 | 27078 | 21 |
| 1210342 | CACTTTCATAATGCTGGC | eeeeeeeeeeeeeeeeee | ssosssssssssssoss | 27061 | 27078 | 21 |
| 1263778 | CACTTTCATAATGCTGGC | eeeeeeeeeeeeeeeeee | sossssssssssssoss | 27061 | 27078 | 21 |
| 1263781 | CACTTTCATAATGCTGGC | eeeeeeeeeeeeeeeeee | sosssssssssosssss | 27061 | 27078 | 21 |
| 1263783 | CACTTTCATAATGCTGGC | eeeeeeeeeeeeeeeeee | sosssssssosssssss | 27061 | 27078 | 21 |
| 1263785 | CACTTTCATAATGCTGGC | eeeeeeeeeeeeeeeeee | sosssssosssssssss | 27061 | 27078 | 21 |
| 1263787 | CACTTTCATAATGCTGGC | eeeeeeeeeeeeeeeeee | sosssosssssssssss | 27061 | 27078 | 21 |
| 1263789 | CACTTTCATAATGCTGGC | eeeeeeeeeeeeeeeeee | sososssssssssssss | 27061 | 27078 | 21 |
| 1263791 | CACTTTCATAATGCTGGC | eeeeeeeeeeeeeeeeee | ssssosssssssssoss | 27061 | 27078 | 21 |
| 1263793 | CACTTTCATAATGCTGGC | eeeeeeeeeeeeeeeeee | ssssssosssssssoss | 27061 | 27078 | 21 |
| 1263795 | CACTTTCATAATGCTGGC | eeeeeeeeeeeeeeeeee | ssssssssosssssoss | 27061 | 27078 | 21 |
| 1263797 | CACTTTCATAATGCTGGC | eeeeeeeeeeeeeeeeee | ssssssssssosssoss | 27061 | 27078 | 21 |
| 1263799 | CACTTTCATAATGCTGGC | eeeeeeeeeeeeeeeeee | ssssssssssssososs | 27061 | 27078 | 21 |
| 1263800 | CACTTTCATAATGCTGGC | eeeeeeeeeeeeeeeeee | soossssssssssssss | 27061 | 27078 | 21 |
| 1263802 | CACTTTCATAATGCTGGC | eeeeeeeeeeeeeeeeee | sssoossssssssssss | 27061 | 27078 | 21 |
| 1263804 | CACTTTCATAATGCTGGC | eeeeeeeeeeeeeeeeee | sssssoossssssssss | 27061 | 27078 | 21 |
| 1263806 | CACTTTCATAATGCTGGC | eeeeeeeeeeeeeeeeee | sssssssoossssssss | 27061 | 27078 | 21 |
| 1263808 | CACTTTCATAATGCTGGC | eeeeeeeeeeeeeeeeee | sssssssssoossssss | 27061 | 27078 | 21 |
| 1263810 | CACTTTCATAATGCTGGC | eeeeeeeeeeeeeeeeee | sssssssssssoossss | 27061 | 27078 | 21 |
| 1263812 | CACTTTCATAATGCTGGC | eeeeeeeeeeeeeeeeee | sssssssssssssooss | 27061 | 27078 | 21 |
| 1210343 | CACTTTCATAATGCTGGC | eeeeeeeeeeeeeeeeee | ssosssssosssssoss | 27061 | 27078 | 21 |
| 1212825 | CACTTTCATAATGCTGGC | eeeeeeeeeeeeeeeeee | sssssssooosssssss | 27061 | 27078 | 21 |
| 1212817 | CACTTTCATAATGCTGGC | eeeeeeeeeeeeeeeeee | soossssssssssooss | 27061 | 27078 | 21 |
| 1212824 | CACTTTCATAATGCTGGC | eeeeeeeeeeeeeeeeee | sssssssoooossssss | 27061 | 27078 | 21 |
| 1212826 | CACTTTCATAATGCTGGC | eeeeeeeeeeeeeeeeee | ssoooosssssssssss | 27061 | 27078 | 21 |
| 1212827 | CACTTTCATAATGCTGGC | eeeeeeeeeeeeeeeeee | ssssoooosssssssss | 27061 | 27078 | 21 |
| 1212828 | CACTTTCATAATGCTGGC | eeeeeeeeeeeeeeeeee | ssssssoooosssssss | 27061 | 27078 | 21 |
| 1212829 | CACTTTCATAATGCTGGC | eeeeeeeeeeeeeeeeee | ssssssssoooosssss | 27061 | 27078 | 21 |
| 1212830 | CACTTTCATAATGCTGGC | eeeeeeeeeeeeeeeeee | ssssssssssoooosss | 27061 | 27078 | 21 |
| 1212831 | CACTTTCATAATGCTGGC | eeeeeeeeeeeeeeeeee | sssssssssssooooss | 27061 | 27078 | 21 |
| 1212818 | CACTTTCATAATGCTGGC | eeeeeeeeeeeeeeeeee | sooosssssssssooss | 27061 | 27078 | 21 |
| 1212823 | CACTTTCATAATGCTGGC | eeeeeeeeeeeeeeeeee | ssssssooooossssss | 27061 | 27078 | 21 |
| 1212819 | CACTTTCATAATGCTGGC | eeeeeeeeeeeeeeeeee | sooossssssssoooss | 27061 | 27078 | 21 |
| 1212822 | CACTTTCATAATGCTGGC | eeeeeeeeeeeeeeeeee | ssssssoooooosssss | 27061 | 27078 | 21 |
| 1212820 | CACTTTCATAATGCTGGC | eeeeeeeeeeeeeeeeee | soooosssssssoooss | 27061 | 27078 | 21 |
| 1212821 | CACTTTCATAATGCTGGC | eeeeeeeeeeeeeeeeee | sssssooooooosssss | 27061 | 27078 | 21 |
| 1287065 | TCACTTTCATAATGCTGGC | eeeeeeeeeeeeeeeeeee | ssssssssssssssssss | 27061 | 27079 | 30 |
| 1210341 | ACTTTCATAATGCTGG | eeeeeeeeeeeeeeee | sssssssssssssss | 27062 | 27077 | 31 |
| 524403 | CACTTTCATAATGCTGG | eeeeeeeeeeeeeeeee | ssssssssssssssss | 27062 | 27078 | 32 |
| 1287121 | TCACTTTCATAATGCTGG | eeeeeeeeeeeeeeeeee | ssssssssssssssoss | 27062 | 27079 | 23 |
| 1287120 | TCACTTTCATAATGCTGG | eeeeeeeeeeeeeeeeee | sssssssssssssosss | 27062 | 27079 | 23 |
| 1287113 | TCACTTTCATAATGCTGG | eeeeeeeeeeeeeeeeee | sssssssssssssooss | 27062 | 27079 | 23 |
| 1287110 | TCACTTTCATAATGCTGG | eeeeeeeeeeeeeeeeee | ssssssssssssososs | 27062 | 27079 | 23 |
| 1287119 | TCACTTTCATAATGCTGG | eeeeeeeeeeeeeeeeee | sssssssssssosssss | 27062 | 27079 | 23 |
| 1364782 | TCACTTTCATAATGCTGG | eeeeeeeeeeeeeeeeee | sssssssssssososss | 27062 | 27079 | 23 |
| 1364777 | TCACTTTCATAATGCTGG | eeeeeeeeeeeeeeeeee | ssssssssssossosss | 27062 | 27079 | 23 |
| 1287118 | TCACTTTCATAATGCTGG | eeeeeeeeeeeeeeeeee | sssssssssosssssss | 27062 | 27079 | 23 |
| 1364783 | TCACTTTCATAATGCTGG | eeeeeeeeeeeeeeeeee | sssssssssosssosss | 27062 | 27079 | 23 |
| 1287109 | TCACTTTCATAATGCTGG | eeeeeeeeeeeeeeeeee | ssssssssosssssoss | 27062 | 27079 | 23 |
| 1364784 | TCACTTTCATAATGCTGG | eeeeeeeeeeeeeeeeee | ssssssssossssosss | 27062 | 27079 | 23 |
| 1287117 | TCACTTTCATAATGCTGG | eeeeeeeeeeeeeeeeee | sssssssosssssssss | 27062 | 27079 | 23 |
| 1287112 | TCACTTTCATAATGCTGG | eeeeeeeeeeeeeeeeee | sssssssoossssssss | 27062 | 27079 | 23 |
| 1287116 | TCACTTTCATAATGCTGG | eeeeeeeeeeeeeeeeee | sssssosssssssssss | 27062 | 27079 | 23 |
| 1287115 | TCACTTTCATAATGCTGG | eeeeeeeeeeeeeeeeee | sssosssssssssssss | 27062 | 27079 | 23 |
| 1287114 | TCACTTTCATAATGCTGG | eeeeeeeeeeeeeeeeee | sosssssssssssssss | 27062 | 27079 | 23 |
| 1287106 | TCACTTTCATAATGCTGG | eeeeeeeeeeeeeeeeee | sossssssssssssoss | 27062 | 27079 | 23 |
| 1287107 | TCACTTTCATAATGCTGG | eeeeeeeeeeeeeeeeee | sossssssossssssss | 27062 | 27079 | 23 |
| 1287108 | TCACTTTCATAATGCTGG | eeeeeeeeeeeeeeeeee | sososssssssssssss | 27062 | 27079 | 23 |
| 1287111 | TCACTTTCATAATGCTGG | eeeeeeeeeeeeeeeeee | soossssssssssssss | 27062 | 27079 | 23 |
| 1287066 | TTCACTTTCATAATGCTGG | eeeeeeeeeeeeeeeeeee | ssssssssssssssssss | 27062 | 27080 | 33 |
| 1287074 | TTCACTTTCATAATGCTGG | eeeeeeeeeeeeeeeeeee | sssssssssssssssoss | 27062 | 27080 | 33 |
| 1287071 | TTCACTTTCATAATGCTGG | eeeeeeeeeeeeeeeeeee | sssssssssssssososs | 27062 | 27080 | 33 |
| 1287073 | TTCACTTTCATAATGCTGG | eeeeeeeeeeeeeeeeeee | ssssssssosssssssss | 27062 | 27080 | 33 |
| 1287070 | TTCACTTTCATAATGCTGG | eeeeeeeeeeeeeeeeeee | ssssssssossssssoss | 27062 | 27080 | 33 |
| 1287072 | TTCACTTTCATAATGCTGG | eeeeeeeeeeeeeeeeeee | sossssssssssssssss | 27062 | 27080 | 33 |
| 1287067 | TTCACTTTCATAATGCTGG | eeeeeeeeeeeeeeeeeee | sosssssssssssssoss | 27062 | 27080 | 33 |
| 1287068 | TTCACTTTCATAATGCTGG | eeeeeeeeeeeeeeeeeee | sossssssosssssssss | 27062 | 27080 | 33 |
| 1287069 | TTCACTTTCATAATGCTGG | eeeeeeeeeeeeeeeeeee | sosossssssssssssss | 27062 | 27080 | 33 |
| 1287060 | ATTCACTTTCATAATGCTGG | eeeeeeeeeeeeeeeeeeee | ssssssssssssssssoss | 27062 | 27081 | 20 |
| 1287057 | ATTCACTTTCATAATGCTGG | eeeeeeeeeeeeeeeeeeee | sssssssssssssssooss | 27062 | 27081 | 20 |
| 1287054 | ATTCACTTTCATAATGCTGG | eeeeeeeeeeeeeeeeeeee | ssssssssssssssososs | 27062 | 27081 | 20 |
| 1287059 | ATTCACTTTCATAATGCTGG | eeeeeeeeeeeeeeeeeeee | sssssssssosssssssss | 27062 | 27081 | 20 |
| 1287053 | ATTCACTTTCATAATGCTGG | eeeeeeeeeeeeeeeeeeee | sssssssssossssssoss | 27062 | 27081 | 20 |
| 1287056 | ATTCACTTTCATAATGCTGG | eeeeeeeeeeeeeeeeeeee | ssssssssoosssssssss | 27062 | 27081 | 20 |
| 1287058 | ATTCACTTTCATAATGCTGG | eeeeeeeeeeeeeeeeeeee | sosssssssssssssssss | 27062 | 27081 | 20 |
| 1287050 | ATTCACTTTCATAATGCTGG | eeeeeeeeeeeeeeeeeeee | sossssssssssssssoss | 27062 | 27081 | 20 |
| 1287051 | ATTCACTTTCATAATGCTGG | eeeeeeeeeeeeeeeeeeee | sosssssssosssssssss | 27062 | 27081 | 20 |
| 1287052 | ATTCACTTTCATAATGCTGG | eeeeeeeeeeeeeeeeeeee | sososssssssssssssss | 27062 | 27081 | 20 |
| 1287055 | ATTCACTTTCATAATGCTGG | eeeeeeeeeeeeeeeeeeee | soossssssssssssssss | 27062 | 27081 | 20 |
| 1287075 | ATTCACTTTCATAATGCTG | eeeeeeeeeeeeeeeeeee | ssssssssssssssssss | 27063 | 27081 | 34 |
| 1287062 | AGATTCACTTTCATAATGCT | eeeeeeeeeeeeeeeeeeee | sssssssssssssssssss | 27064 | 27083 | 35 |
| 1287061 | GATTCACTTTCATAATGCTG | eeeeeeeeeeeeeeeeeeee | sssssssssssssssssss | 27063 | 27082 | 49 |
| 1287076 | GATTCACTTTCATAATGCT | eeeeeeeeeeeeeeeeeee | ssssssssssssssssss | 27064 | 27082 | 50 |
| 1287701 | TCACTTTCATAATGCTGGT | eeeeeeeeeeeeeeeeeee | ssssssssssssssssss | 27062 | 27079 | 36 |
| 1287702 | TCACTTTCATAATGCTGGA | eeeeeeeeeeeeeeeeeee | ssssssssssssssssss | 27062 | 27079 | 37 |

### Table 3

The modified oligonucleotides in Table 3 below are 16, 17, 18, 19 or 20 nucleosides in length. Each nucleoside comprises a 2′-NMA sugar moiety. The sugar motif for each modified oligonucleotide is provided in the Sugar Motif column, wherein each ‘n’ represents a 2′-NMA sugar moiety. Each internucleoside linkage is either a phosphorothioate internucleoside linkage or a phosphodiester internucleoside linkage. The internucleoside linkage motif for each modified oligonucleotide is provided in the Internucleoside Linkage Motif column, wherein each ‘s’ represents a phosphorothioate internucleoside linkage, and each ‘o’ represents a phosphodiester internucleoside linkage. Each cytosine is a 5-methyl cytosine.

Each modified oligonucleotide listed in Table 3 below is 100% complementary to SEQ ID NO: 1 (GENBANK Accession No. NT_006713.14 truncated from nucleotides 19939708 to 19967777). “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

TABLE 3
2′-NMA modified oligonucleotides with PS or mixed PS/PO internucleoside linkages

| | | | | SEQ | SEQ | |
|---|---|---|---|---|---|---|
| | | | | ID | ID | |
| | | | Internucleoside | No: 1 | No: 1 | SEQ |
| Compound | Nucleobase Sequence | Sugar Motif | Linkage Motif | Start | Stop | ID |
| Number | (5′ to 3′) | (5′ to 3′) | (5′ to 3′) | Site | Site | No. |
| 1287127 | CACTTTCATAATGCTGGCA | nnnnnnnnnnnnnnnnnnn | ssssssssssssssssss | 27060 | 27078 | 26 |
| 1287122 | TCACTTTCATAATGCTGGCA | nnnnnnnnnnnnnnnnnnnn | sssssssssssssssssss | 27060 | 27079 | 27 |
| 1212871 | CTTTCATAATGCTGGC | nnnnnnnnnnnnnnnn | sssssssssssssss | 27061 | 27076 | 28 |
| 1212869 | ACTTTCATAATGCTGGC | nnnnnnnnnnnnnnnnn | ssssssssssssssss | 27061 | 27077 | 29 |
| 1358996 | CACTTTCATAATGCTGGC | nnnnnnnnnnnnnnnnnn | sososssssssssssss | 27061 | 27078 | 21 |
| 1212873 | CACTTTCATAATGCTGGC | nnnnnnnnnnnnnnnnnn | ssosssssssssssoss | 27061 | 27078 | 21 |
| 1212874 | CACTTTCATAATGCTGGC | nnnnnnnnnnnnnnnnnn | ssosssssosssssoss | 27061 | 27078 | 21 |
| 1212875 | CACTTTCATAATGCTGGC | nnnnnnnnnnnnnnnnnn | ssosssosssosssoss | 27061 | 27078 | 21 |
| 1212879 | CACTTTCATAATGCTGGC | nnnnnnnnnnnnnnnnnn | soossssssssssooss | 27061 | 27078 | 21 |
| 1212880 | CACTTTCATAATGCTGGC | nnnnnnnnnnnnnnnnnn | sooosssssssssooss | 27061 | 27078 | 21 |
| 1212881 | CACTTTCATAATGCTGGC | nnnnnnnnnnnnnnnnnn | sooossssssssoooss | 27061 | 27078 | 21 |
| 1212885 | CACTTTCATAATGCTGGC | nnnnnnnnnnnnnnnnnn | ssssssooooossssss | 27061 | 27078 | 21 |
| 1212887 | CACTTTCATAATGCTGGC | nnnnnnnnnnnnnnnnnn | sssssssooosssssss | 27061 | 27078 | 21 |
| 1287128 | TCACTTTCATAATGCTGGC | nnnnnnnnnnnnnnnnnnn | ssssssssssssssssss | 27061 | 27079 | 30 |
| 1212870 | CACTTTCATAATGCTGG | nnnnnnnnnnnnnnnnn | ssssssssssssssss | 27062 | 27078 | 32 |
| 1287132 | TCACTTTCATAATGCTGG | nnnnnnnnnnnnnnnnnn | sossssssssssssoss | 27062 | 27079 | 23 |
| 1287133 | TCACTTTCATAATGCTGG | nnnnnnnnnnnnnnnnnn | ssssssssossssssss | 27062 | 27079 | 23 |
| 1332246 | TCACTTTCATAATGCTGG | nnnnnnnnnnnnnnnnnn | ssssssssosssssoss | 27062 | 27079 | 23 |
| 1332265 | TCACTTTCATAATGCTGG | nnnnnnnnnnnnnnnnnn | ssssssssssssososs | 27062 | 27079 | 23 |
| 1364778 | TCACTTTCATAATGCTGG | nnnnnnnnnnnnnnnnnn | sssssssssssososss | 27062 | 27079 | 23 |
| 1364779 | TCACTTTCATAATGCTGG | nnnnnnnnnnnnnnnnnn | ssssssssssossosss | 27062 | 27079 | 23 |
| 1364780 | TCACTTTCATAATGCTGG | nnnnnnnnnnnnnnnnnn | sssssssssosssosss | 27062 | 27079 | 23 |
| 1364781 | TCACTTTCATAATGCTGG | nnnnnnnnnnnnnnnnnn | ssssssssossssosss | 27062 | 27079 | 23 |
| 1287129 | TTCACTTTCATAATGCTGG | nnnnnnnnnnnnnnnnnnn | ssssssssssssssssss | 27062 | 27080 | 33 |
| 1287130 | TTCACTTTCATAATGCTGG | nnnnnnnnnnnnnnnnnnn | sosssssssssssssoss | 27062 | 27080 | 33 |
| 1287131 | TTCACTTTCATAATGCTGG | nnnnnnnnnnnnnnnnnnn | ssssssssosssssssss | 27062 | 27080 | 33 |
| 1332263 | TTCACTTTCATAATGCTGG | nnnnnnnnnnnnnnnnnnn | ssssssssossssssoss | 27062 | 27080 | 33 |
| 1332264 | TTCACTTTCATAATGCTGG | nnnnnnnnnnnnnnnnnnn | sssssssssssssssoss | 27062 | 27080 | 33 |
| 1332266 | TTCACTTTCATAATGCTGG | nnnnnnnnnnnnnnnnnnn | sssssssssssssososs | 27062 | 27080 | 33 |
| 1332270 | TTCACTTTCATAATGCTGG | nnnnnnnnnnnnnnnnnnn | ssssssssssssssooss | 27062 | 27080 | 33 |
| 1287124 | ATTCACTTTCATAATGCTGG | nnnnnnnnnnnnnnnnnnnn | sssssssssssssssssss | 27062 | 27081 | 20 |
| 1287125 | ATTCACTTTCATAATGCTGG | nnnnnnnnnnnnnnnnnnnn | sossssssssssssssoss | 27062 | 27081 | 20 |
| 1287126 | ATTCACTTTCATAATGCTGG | nnnnnnnnnnnnnnnnnnnn | sssssssssosssssssss | 27062 | 27081 | 20 |
| 1332267 | ATTCACTTTCATAATGCTGG | nnnnnnnnnnnnnnnnnnnn | ssssssssssssssssoss | 27062 | 27081 | 20 |
| 1332268 | ATTCACTTTCATAATGCTGG | nnnnnnnnnnnnnnnnnnnn | sssssssssssssssooss | 27062 | 27081 | 20 |
| 1332269 | ATTCACTTTCATAATGCTGG | nnnnnnnnnnnnnnnnnnnn | ssssssssssssssososs | 27062 | 27081 | 20 |
| 1332271 | ATTCACTTTCATAATGCTGG | nnnnnnnnnnnnnnnnnnnn | sssssssssossssssoss | 27062 | 27081 | 20 |
| 1287123 | TTCACTTTCATAATGCTGGC | nnnnnnnnnnnnnnnnnnnn | sssssssssssssssssss | 27061 | 27080 | 22 |

### Table 4

The modified oligonucleotides in Table 4 below are 18 or 19 nucleosides in length. Each nucleoside comprises either a 2′-MOE sugar moiety or a 2′-NMA sugar moiety. The sugar motif for each modified oligonucleotide is provided in the Sugar Motif column, wherein each ‘e’ represents a 2′-MOE sugar moiety, and each ‘n’ represents a 2′-NMA sugar moiety. Each internucleoside linkage is a phosphorothioate internucleoside linkage. The internucleoside linkage motif for each modified oligonucleotide is provided in the Internucleoside Linkage Motif column, wherein each ‘s’ represents a phosphorothioate internucleoside linkage. Each cytosine is a 5-methyl cytosine.

Each modified oligonucleotide listed in Table 4 below is 100% complementary to SEQ ID NO: 1 (GENBANK Accession No. NT_006713.14 truncated from nucleotides 19939708 to 19967777), unless specifically stated otherwise. Non-complementary nucleobases are specified in the Nucleobase Sequence column in underlined, bold, italicized font. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

TABLE 4
Mixed 2′-MOE/2′-NMA modified oligonucleotides with PS internucleoside linkages

| | | | | SEQ | SEQ | |
|---|---|---|---|---|---|---|
| | | | | ID | ID | |
| | | | Internucleoside | No: 1 | No: 1 | SEQ |
| Compound | Nucleobase Sequence | Sugar Motif | Linkage Motif | Start | Stop | ID |
| Number | (5′ to 3′) | (5′ to 3′) | (5′ to 3′) | Site | Site | No. |
| 1212931 | CACTTTCATAATGCTGGC | nennnnneneennnnnnn | sssssssssssssssss | 27061 | 27078 | 21 |
| 1212936 | CACTTTCATAATGCTGGC | nnnnnnnnnmmnenneen | sssssssssssssssss | 27061 | 27078 | 21 |
| 1212941 | CACTTTCATAATGCTGGC | nennnnneneenenneen | sssssssssssssssss | 27061 | 27078 | 21 |
| 1287728 | TCACTTTCATAATGCTGGC | nnnnnnnnnnnnnnnnnne | ssssssssssssssssss | 27061 | 27079 | 30 |
| 1287729 | TCACTTTCATAATGCTGGT | nnnnnnnnnnnnnnnnnne | ssssssssssssssssss | 27062 | 27079 | 36 |
| 1287730 | TCACTTTCATAATGCTGGA | nnnnnnnnnnnnnnnnnne | ssssssssssssssssss | 27062 | 27079 | 37 |

### Table 5

The modified oligonucleotides in Table 5 below are 16, 17, or 18 nucleosides in length. Each nucleoside comprises either a 2′-MOE sugar moiety or a cEt sugar moiety. The sugar motif for each modified oligonucleotide is provided in the Sugar Motif column, wherein each ‘e’ represents a 2′-MOE sugar moiety, and each ‘k’ represents a cEt sugar moiety. Each internucleoside linkage is a phosphorothioate internucleoside linkage. The internucleoside linkage motif for each modified oligonucleotide is provided in the Internucleoside Linkage Motif column, wherein each ‘s’ represents a phosphorothioate internucleoside linkage. Each cytosine is a 5-methyl cytosine.

Each modified oligonucleotide listed in Table 5 below is 100% complementary to SEQ ID NO: 1 (GENBANK Accession No. NT_006713.14 truncated from nucleotides 19939708 to 19967777). “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

TABLE 5
Mixed 2′-MOE/cEt modified oligonucleotides with PS internucleoside linkages

| | | | | SEQ | SEQ | |
|---|---|---|---|---|---|---|
| | | | | ID | ID | |
| | | | Internucleoside | No: 1 | No: 1 | SEQ |
| Compound | Nucleobase Sequence | Sugar Motif | Linkage Motif | Start | Stop | ID |
| Number | (5′ to 3′) | (5′ to 3′) | (5′ to 3′) | Site | Site | No. |
| 1212961 | CACTTTCATAATGCTGGC | keekeekeekeekeeeek | sssssssssssssssss | 27061 | 27078 | 21 |
| 1212962 | CACTTTCATAATGCTGGC | keeekeeekeeekeeeek | sssssssssssssssss | 27061 | 27078 | 21 |
| 1212963 | CACTTTCATAATGCTGGC | keeeeekeeeeekeeeek | sssssssssssssssss | 27061 | 27078 | 21 |
| 1212964 | CACTTTCATAATGCTGGC | keeeeeeekeeeeeeeek | sssssssssssssssss | 27061 | 27078 | 21 |
| 1212965 | CACTTTCATAATGCTGGC | keeeeeeeeeeeeeeeek | sssssssssssssssss | 27061 | 27078 | 21 |
| 1212966 | CACTTTCATAATGCTGGC | eeekeekeekeekeekek | sssssssssssssssss | 27061 | 27078 | 21 |
| 1212967 | CACTTTCATAATGCTGGC | eeekeekeekeekeekee | sssssssssssssssss | 27061 | 27078 | 21 |
| 1212968 | CACTTTCATAATGCTGGC | eeeeeekeekeekeekee | sssssssssssssssss | 27061 | 27078 | 21 |
| 1212969 | CACTTTCATAATGCTGGC | eeeeeekeekeekeeeee | sssssssssssssssss | 27061 | 27078 | 21 |
| 1212970 | CACTTTCATAATGCTGGC | eeeeeekeeeeekeeeee | sssssssssssssssss | 27061 | 27078 | 21 |
| 1212971 | CACTTTCATAATGCTGGC | keekeekeekeeeeeeee | sssssssssssssssss | 27061 | 27078 | 21 |
| 1212972 | CACTTTCATAATGCTGGC | eeeeeeeekeekeekeek | sssssssssssssssss | 27061 | 27078 | 21 |
| 1212973 | CACTTTCATAATGCTGGC | keekeekeeeeeeeeeee | sssssssssssssssss | 27061 | 27078 | 21 |
| 1212974 | CACTTTCATAATGCTGGC | eeeeeeeeeeekeekeek | sssssssssssssssss | 27061 | 27078 | 21 |
| 1212975 | CACTTTCATAATGCTGGC | keekeeeeeeeeeeeeee | sssssssssssssssss | 27061 | 27078 | 21 |
| 1212976 | CACTTTCATAATGCTGGC | eeeeeeeeeeeeeekeek | sssssssssssssssss | 27061 | 27078 | 21 |
| 1212977 | ACTTTCATAATGCTGGC | keekeekeekeekeeek | ssssssssssssssss | 27061 | 27077 | 29 |
| 1212978 | ACTTTCATAATGCTGGC | keeekeeekeeekeeek | ssssssssssssssss | 27061 | 27077 | 29 |
| 1212979 | ACTTTCATAATGCTGGC | keeeekeeeeekeeeek | ssssssssssssssss | 27061 | 27077 | 29 |
| 1212980 | ACTTTCATAATGCTGGC | keeeeeeekeeeeeeek | ssssssssssssssss | 27061 | 27077 | 29 |
| 1212981 | ACTTTCATAATGCTGGC | keeeeeeeeeeeeeeek | ssssssssssssssss | 27061 | 27077 | 29 |
| 1212982 | ACTTTCATAATGCTGGC | eekeekeekeekeekek | ssssssssssssssss | 27061 | 27077 | 29 |
| 1212983 | ACTTTCATAATGCTGGC | eekeekeekeekeekee | ssssssssssssssss | 27061 | 27077 | 29 |
| 1212984 | ACTTTCATAATGCTGGC | eeeeekeekeekeekee | ssssssssssssssss | 27061 | 27077 | 29 |
| 1212985 | ACTTTCATAATGCTGGC | eeeeekeekeekeeeee | ssssssssssssssss | 27061 | 27077 | 29 |
| 1212986 | ACTTTCATAATGCTGGC | eeeeekeeeeekeeeee | ssssssssssssssss | 27061 | 27077 | 29 |
| 1212987 | ACTTTCATAATGCTGGC | keekeekeekeeeeeee | ssssssssssssssss | 27061 | 27077 | 29 |
| 1212988 | ACTTTCATAATGCTGGC | eeeeeeekeekeekeek | ssssssssssssssss | 27061 | 27077 | 29 |
| 1212989 | ACTTTCATAATGCTGGC | keekeekeeeeeeeeee | ssssssssssssssss | 27061 | 27077 | 29 |
| 1212990 | ACTTTCATAATGCTGGC | eeeeeeeeeekeekeek | ssssssssssssssss | 27061 | 27077 | 29 |
| 1212991 | ACTTTCATAATGCTGGC | keekeeeeeeeeeeeee | ssssssssssssssss | 27061 | 27077 | 29 |
| 1212992 | ACTTTCATAATGCTGGC | eeeeeeeeeeeeekeek | ssssssssssssssss | 27061 | 27077 | 29 |
| 1212993 | CTTTCATAATGCTGGC | keekeekeekeekeek | sssssssssssssss | 27061 | 27076 | 28 |
| 1212994 | CTTTCATAATGCTGGC | keeekeeekeeekeek | sssssssssssssss | 27061 | 27076 | 28 |
| 1212995 | CTTTCATAATGCTGGC | keeeekeeeekeeeek | sssssssssssssss | 27061 | 27076 | 28 |
| 1212996 | CTTTCATAATGCTGGC | keeeeeeekeeeeeek | sssssssssssssss | 27061 | 27076 | 28 |
| 1212997 | CTTTCATAATGCTGGC | keeeeeeeeeeeeeek | sssssssssssssss | 27061 | 27076 | 28 |
| 1212998 | CTTTCATAATGCTGGC | kekeekeekeekeeke | sssssssssssssss | 27061 | 27076 | 28 |
| 1212999 | CTTTCATAATGCTGGC | eekeekeekeekeeke | sssssssssssssss | 27061 | 27076 | 28 |
| 1213000 | CTTTCATAATGCTGGC | eeeeekeekeekeeke | sssssssssssssss | 27061 | 27076 | 28 |
| 1213001 | CTTTCATAATGCTGGC | eeeeekeekeekeeee | sssssssssssssss | 27061 | 27076 | 28 |
| 1213002 | CTTTCATAATGCTGGC | eeeeekeeeeekeeee | sssssssssssssss | 27061 | 27076 | 28 |
| 1213003 | CTTTCATAATGCTGGC | keekeekeekeeeeee | sssssssssssssss | 27061 | 27076 | 28 |
| 1213004 | CTTTCATAATGCTGGC | eeeeeekeekeekeek | sssssssssssssss | 27061 | 27076 | 28 |
| 1213005 | CTTTCATAATGCTGGC | keekeekeeeeeeeee | sssssssssssssss | 27061 | 27076 | 28 |
| 1213006 | CTTTCATAATGCTGGC | eeeeeeeeekeekeek | sssssssssssssss | 27061 | 27076 | 28 |
| 1213007 | CTTTCATAATGCTGGC | keekeeeeeeeeeeee | sssssssssssssss | 27061 | 27076 | 28 |
| 1213008 | CTTTCATAATGCTGGC | eeeeeeeeeeeekeek | sssssssssssssss | 27061 | 27076 | 28 |

### Table 6

The modified oligonucleotides in Table 6 below are 19 or 20 nucleosides in length. Each nucleoside comprises a 2′-MOE sugar moiety, a 2′-NMA sugar moiety, a 2′-OMe sugar moiety, or a 2′--D-deoxyribosyl sugar moiety. The sugar motif for each modified oligonucleotide is provided in the Sugar Motif column, wherein each ‘e’ represents a 2′-MOE sugar moiety, each ‘n’ represents a 2′-NMA sugar moiety, each ‘y’ represents a 2′-OMe sugar moiety, and each ‘d’ represents a 2′-β-D-deoxyribosyl sugar moiety. Each internucleoside linkage is either a phosphorothioate internucleoside linkage or a phosphodiester internucleoside linkage. The internucleoside linkage motif for each modified oligonucleotide, provided in the Internucleoside Linkage Motif column, is (from 5′ to 3′): ssssssssssssssssso; wherein each ‘s’ represents a phosphorothioate internucleoside linkage, and each ‘o’ represents a phosphodiester internucleoside linkage. Cytosines are either non-methylated cytosines or 5-methyl cytosines, wherein each lowercase ‘c’ in the Nucleobase Sequence column represents a non-methylated cytosine, and each uppercase ‘C’ in the Nucleobase Sequence column represents a 5-methyl cytosine.

Each nucleobase in the modified oligonucleotides listed in Table 6 below is complementary to SEQ ID NO: 1 (GENBANK Accession No. NT_006713.14 truncated from nucleotides 19939708 to 19967777), unless specifically stated otherwise. Non-complementary nucleobases are specified in the Nucleobase Sequence column in underlined, bold, italicized font. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

TABLE 6
Modified oligonucleotides with mixed PS/PO internucleoside linkages

| | | | | SEQ | SEQ | |
|---|---|---|---|---|---|---|
| | | | | ID | ID | |
| | | | Internucleoside | No: 1 | No: 1 | SEQ |
| Compound | Nucleobase Sequence | Sugar Motif | Linkage Motif | Start | Stop | ID |
| Number | (5′ to 3′) | (5′ to 3′) | (5′ to 3′) | Site | Site | No. |
| 1287707 | TCACTTTCATAATGCTGGC | eeeeeeeeeeeeeeeeeed | ssssssssssssssssso | 27061 | 27079 | 30 |
| 1287708 | TCACTTTCATAATGCTGGc | eeeeeeeeeeeeeeeeeed | ssssssssssssssssso | 27061 | 27079 | 30 |
| 1287709 | TCACTTTCATAATGCTGGT | eeeeeeeeeeeeeeeeeed | ssssssssssssssssso | 27062 | 27079 | 36 |
| 1287710 | TCACTTTCATAATGCTGGA | eeeeeeeeeeeeeeeeeed | ssssssssssssssssso | 27062 | 27079 | 37 |
| 1287711 | TCACTTTCATAATGCTGGc | eeeeeeeeeeeeeeeeeey | ssssssssssssssssso | 27061 | 27079 | 30 |
| 1287712 | TCACTTTCATAATGCTGGU | eeeeeeeeeeeeeeeeeey | ssssssssssssssssso | 27062 | 27079 | 38 |
| 1287713 | TCACTTTCATAATGCTGGA | eeeeeeeeeeeeeeeeeey | ssssssssssssssssso | 27062 | 27079 | 37 |
| 1287731 | TCACTTTCATAATGCTGGC | nnnnnnnnnnnnnnnnnne | ssssssssssssssssso | 27061 | 27079 | 30 |
| 1287732 | TCACTTTCATAATGCTGGc | nnnnnnnnnnnnnnnnnne | ssssssssssssssssso | 27061 | 27079 | 30 |
| 1287733 | TCACTTTCATAATGCTGGT | nnnnnnnnnnnnnnnnnne | ssssssssssssssssso | 27062 | 27079 | 36 |
| 1287734 | TCACTTTCATAATGCTGGA | nnnnnnnnnnnnnnnnnne | ssssssssssssssssso | 27062 | 27079 | 37 |
| 1287735 | TCACTTTCATAATGCTGGC | nnnnnnnnnnnnnnnnnnd | ssssssssssssssssso | 27061 | 27079 | 30 |
| 1287736 | TCACTTTCATAATGCTGGc | nnnnnnnnnnnnnnnnnnd | ssssssssssssssssso | 27061 | 27079 | 30 |
| 1287737 | TCACTTTCATAATGCTGGT | nnnnnnnnnnnnnnnnnnd | ssssssssssssssssso | 27062 | 27079 | 36 |
| 1287738 | TCACTTTCATAATGCTGGA | nnnnnnnnnnnnnnnnnnd | ssssssssssssssssso | 27062 | 27079 | 37 |
| 1287739 | TCACTTTCATAATGCTGGc | nnnnnnnnnnnnnnnnnny | ssssssssssssssssso | 27061 | 27079 | 30 |
| 1287740 | TCACTTTCATAATGCTGGU | nnnnnnnnnnnnnnnnnny | ssssssssssssssssso | 27062 | 27079 | 38 |
| 1287741 | TCACTTTCATAATGCTGGA | nnnnnnnnnnnnnnnnnny | ssssssssssssssssso | 27062 | 27079 | 37 |
| 1287705 | TCACTTTCATAATGCTGGT | eeeeeeeeeeeeeeeeeee | ssssssssssssssssso | 27062 | 27079 | 36 |
| 1287706 | TCACTTTCATAATGCTGGA | eeeeeeeeeeeeeeeeeee | ssssssssssssssssso | 27062 | 27079 | 37 |
| 1287704 | TCACTTTCATAATGCTGGc | eeeeeeeeeeeeeeeeeee | ssssssssssssssssso | 27061 | 27079 | 30 |
| 1287703 | TCACTTTCATAATGCTGGC | eeeeeeeeeeeeeeeeeee | ssssssssssssssssso | 27061 | 27079 | 30 |

### Table 7

The modified oligonucleotides in Table 7 below are 19 or 20 nucleosides in length. Each nucleoside comprises a 2′-MOE sugar moiety, a 2′-NMA sugar moiety, or a 2′-β-D-deoxyribosyl sugar moiety. The sugar motif for each modified oligonucleotide is provided in the Sugar Motif column, wherein each ‘e’ represents a 2′-MOE sugar moiety, each ‘n’ represents a 2′-NMA sugar moiety, and each ‘d’ represents a 2′-β-D-deoxyribosyl sugar moiety. Each internucleoside linkage is either a phosphorothioate internucleoside linkage or a phosphodiester internucleoside linkage. The internucleoside linkage motif for each modified oligonucleotide, provided in the Internucleoside Linkage Motif column, is (from 5′ to 3′): sssssssssssssssssoo; wherein each ‘s’ represents a phosphorothioate internucleoside linkage, and each ‘o’ represents a phosphodiester internucleoside linkage. Each cytosine is a 5-methyl cytosine. Each nucleobase in the modified oligonucleotide listed in Table 6 below is complementary to SEQ ID NO: 1 (GENBANK Accession No. NT_006713.14 truncated from nucleotides 19939708 to 19967777), unless specifically stated otherwise. Non-complementary nucleobases are specified in the Nucleobase Sequence column in underlined, bold, italicized font. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

TABLE 7
Modified oligonucleotides with mixed PS/PO internucleoside linkages

| | | | | SEQ | SEQ | |
|---|---|---|---|---|---|---|
| | | | | ID | ID | |
| | | | Internucleoside | No: 1 | No: 1 | SEQ |
| Compound | Nucleobase Sequence | Sugar Motif | Linkage Motif | Start | Stop | ID |
| Number | (5 to 3′) | (5′ to 3′) | (5′ to 3′) | Site | Site | No. |
| 1318749 | TCACTTTCATAATGCTGGAA | nnnnnnnnnnnnnnnnnndd | sssssssssssssssssoo | 27062 | 27079 | 39 |
| 1318750 | TCACTTTCATAATGCTGGCA | nnnnnnnnnnnnnnnnnned | sssssssssssssssssoo | 27060 | 27079 | 27 |
| 1318751 | TCACTTTCATAATGCTGGCA | nnnnnnnnnnnnnnnnnndd | sssssssssssssssssoo | 27060 | 27079 | 27 |
| 1318752 | TCACTTTCATAATGCTGGAA | nnnnnnnnnnnnnnnnnned | sssssssssssssssssoo | 27062 | 27079 | 39 |
| 1318753 | TCACTTTCATAATGCTGGAA | nnnnnnnnnnnnnnnnnnde | sssssssssssssssssoo | 27062 | 27079 | 39 |
| 1318754 | TCACTTTCATAATGCTGGCA | nnnnnnnnnnnnnnnnnnde | sssssssssssssssssoo | 27060 | 27079 | 27 |
| 1318755 | TCACTTTCATAATGCTGGAA | nnnnnnnnnnnnnnnnnnee | sssssssssssssssssoo | 27062 | 27079 | 39 |
| 1318756 | TCACTTTCATAATGCTGGCA | nnnnnnnnnnnnnnnnnnee | sssssssssssssssssoo | 27060 | 27079 | 27 |
| 1318757 | TCACTTTCATAATGCTGGAT | eeeeeeeeeeeeeeeeeedd | sssssssssssssssssoo | 27062 | 27079 | 40 |
| 1318758 | TCACTTTCATAATGCTGGAC | eeeeeeeeeeeeeeeeeedd | sssssssssssssssssoo | 27062 | 27079 | 41 |
| 1318759 | TCACTTTCATAATGCTGGTC | eeeeeeeeeeeeeeeeeedd | sssssssssssssssssoo | 27062 | 27079 | 42 |
| 1318760 | TCACTTTCATAATGCTGGAA | eeeeeeeeeeeeeeeeeedd | sssssssssssssssssoo | 27062 | 27079 | 39 |
| 1318761 | TCACTTTCATAATGCTGGTT | eeeeeeeeeeeeeeeeeedd | sssssssssssssssssoo | 27062 | 27079 | 43 |
| 1318762 | TCACTTTCATAATGCTGGTA | eeeeeeeeeeeeeeeeeedd | sssssssssssssssssoo | 27062 | 27079 | 44 |
| 1318763 | TCACTTTCATAATGCTGGCC | eeeeeeeeeeeeeeeeeedd | sssssssssssssssssoo | 27061 | 27079 | 45 |
| 1318764 | TCACTTTCATAATGCTGGAA | eeeeeeeeeeeeeeeeeeed | sssssssssssssssssoo | 27062 | 27079 | 39 |
| 1318765 | TCACTTTCATAATGCTGGAC | eeeeeeeeeeeeeeeeeede | sssssssssssssssssoo | 27062 | 27079 | 41 |
| 1318766 | TCACTTTCATAATGCTGGCT | eeeeeeeeeeeeeeeeeedd | sssssssssssssssssoo | 27061 | 27079 | 46 |
| 1318767 | TCACTTTCATAATGCTGGTT | eeeeeeeeeeeeeeeeeede | sssssssssssssssssoo | 27062 | 27079 | 43 |
| 1318768 | TCACTTTCATAATGCTGGCA | eeeeeeeeeeeeeeeeeedd | sssssssssssssssssoo | 27060 | 27079 | 27 |
| 1318769 | TCACTTTCATAATGCTGGCA | eeeeeeeeeeeeeeeeeeed | sssssssssssssssssoo | 27060 | 27079 | 27 |
| 1318770 | TCACTTTCATAATGCTGGAT | eeeeeeeeeeeeeeeeeede | sssssssssssssssssoo | 27062 | 27079 | 40 |
| 1318771 | TCACTTTCATAATGCTGGTA | eeeeeeeeeeeeeeeeeede | sssssssssssssssssoo | 27062 | 27079 | 44 |
| 1318772 | TCACTTTCATAATGCTGGAA | eeeeeeeeeeeeeeeeeede | sssssssssssssssssoo | 27062 | 27079 | 39 |
| 1318773 | TCACTTTCATAATGCTGGTC | eeeeeeeeeeeeeeeeeede | sssssssssssssssssoo | 27062 | 27079 | 42 |
| 1318774 | TCACTTTCATAATGCTGGCC | eeeeeeeeeeeeeeeeeede | sssssssssssssssssoo | 27061 | 27079 | 45 |
| 1318775 | TCACTTTCATAATGCTGGCT | eeeeeeeeeeeeeeeeeede | sssssssssssssssssoo | 27061 | 27079 | 46 |
| 1318776 | TCACTTTCATAATGCTGGCA | eeeeeeeeeeeeeeeeeede | sssssssssssssssssoo | 27060 | 27079 | 27 |
| 1333508 | TCACTTTCATAATGCTGGCT | nnnnnnnnnnnnnnnnnnee | sssssssssssssssssoo | 27061 | 27079 | 46 |
| 1318777 | TCACTTTCATAATGCTGGAC | eeeeeeeeeeeeeeeeeeee | sssssssssssssssssoo | 27062 | 27079 | 41 |
| 1318778 | TCACTTTCATAATGCTGGTT | eeeeeeeeeeeeeeeeeeee | sssssssssssssssssoo | 27062 | 27079 | 43 |
| 1318779 | TCACTTTCATAATGCTGGAA | eeeeeeeeeeeeeeeeeeee | sssssssssssssssssoo | 27062 | 27079 | 39 |
| 1318780 | TCACTTTCATAATGCTGGTC | eeeeeeeeeeeeeeeeeeee | sssssssssssssssssoo | 27062 | 27079 | 42 |
| 1318781 | TCACTTTCATAATGCTGGAT | eeeeeeeeeeeeeeeeeeee | sssssssssssssssssoo | 27062 | 27079 | 40 |
| 1318782 | TCACTTTCATAATGCTGGCT | eeeeeeeeeeeeeeeeeeee | sssssssssssssssssoo | 27061 | 27079 | 46 |
| 1318783 | TCACTTTCATAATGCTGGTA | eeeeeeeeeeeeeeeeeeee | sssssssssssssssssoo | 27062 | 27079 | 44 |
| 1318784 | TCACTTTCATAATGCTGGCC | eeeeeeeeeeeeeeeeeeee | sssssssssssssssssoo | 27061 | 27079 | 45 |
| 1318748 | TCACTTTCATAATGCTGGCA | eeeeeeeeeeeeeeeeeeee | sssssssssssssssssoo | 27060 | 27079 | 27 |

### Table 8

The modified oligonucleotides in Table 8 below are each 19 nucleosides in length. Each nucleoside comprises a 2′-MOE sugar moiety, a 2′-NMA sugar moiety, or a 2′-β-D-deoxyribosyl sugar moiety. The sugar motif for each modified oligonucleotide is provided in the Sugar Motif column, wherein each ‘e’ represents a 2′-MOE sugar moiety, each ‘n’ represents a 2′-NMA sugar moiety, and each ‘d’ represents a 2′-β-D-deoxyribosyl sugar moiety. Each internucleoside linkage is either a phosphorothioate internucleoside linkage or a phosphodiester internucleoside linkage. The internucleoside linkage motif for each modified oligonucleotide, provided in the Internucleoside Linkage Motif column, is (from 5′ to 3′): ssssssssssssososso; wherein each ‘s’ represents a phosphorothioate internucleoside linkage, and each ‘o’ represents a phosphodiester internucleoside linkage. Each cytosine is a 5-methyl cytosine. Each nucleobase in the modified oligonucleotide listed in Table 8 below is complementary to SEQ ID NO: 1 (GENBANK Accession No. NT_006713.14 truncated from nucleotides 19939708 to 19967777), unless specifically stated otherwise. Non-complementary nucleobases are specified in the Nucleobase Sequence column in underlined, bold, italicized font. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

TABLE 8
Modified oligonucleotides with mixed PS/PO internucleoside linkages

| | | | | SEQ | SEQ | |
|---|---|---|---|---|---|---|
| | | | | ID | ID | |
| | | | Internucleoside | No: 1 | No: 1 | SEQ |
| Compound | Nucleobase Sequence | Sugar Motif | Linkage Motif | Start | Stop | ID |
| Number | (5′ to 3′) | (5′ to 3′) | (5′ to 3′) | Site | Site | No. |
| 1332247 | TCACTTTCATAATGCTGGC | nnnnnnnnnnnnnnnnnnd | ssssssssssssososso | 27061 | 27079 | 30 |
| 1332248 | TCACTTTCATAATGCTGGA | nnnnnnnnnnnnnnnnnnd | ssssssssssssososso | 27062 | 27079 | 37 |
| 1332249 | TCACTTTCATAATGCTGGA | nnnnnnnnnnnnnnnnnne | ssssssssssssososso | 27062 | 27079 | 37 |
| 1332251 | TCACTTTCATAATGCTGGC | nnnnnnnnnnnnnnnnnne | ssssssssssssososso | 27061 | 27079 | 30 |
| 1332255 | TCACTTTCATAATGCTGGC | eeeeeeeeeeeeeeeeeed | ssssssssssssososso | 27061 | 27079 | 30 |
| 1332257 | TCACTTTCATAATGCTGGA | eeeeeeeeeeeeeeeeeed | ssssssssssssososso | 27062 | 27079 | 37 |
| 1332256 | TCACTTTCATAATGCTGGA | eeeeeeeeeeeeeeeeeee | ssssssssssssososso | 27062 | 27079 | 37 |
| 1332258 | TCACTTTCATAATGCTGGC | eeeeeeeeeeeeeeeeeee | ssssssssssssososso | 27061 | 27079 | 30 |

### Table 9

The modified oligonucleotides in Table 9 below are each 19 nucleosides in length. Each nucleoside comprises a 2′-MOE sugar moiety, a 2′-NMA sugar moiety, or a 2′-β-D-deoxyribosyl sugar moiety. The sugar motif for each modified oligonucleotide is provided in the Sugar Motif column, wherein each ‘e’ represents a 2′-MOE sugar moiety, each ‘n’ represents a 2′-NMA sugar moiety, and each ‘d’ represents a 2′-β-D-deoxyribosyl sugar moiety. Each internucleoside linkage is either a phosphorothioate internucleoside linkage or a phosphodiester internucleoside linkage. The internucleoside linkage motif for each modified oligonucleotide, provided in the Internucleoside Linkage Motif column, is (from 5′ to 3′): ssssssssssssssosso; wherein each ‘s’ represents a phosphorothioate internucleoside linkage, and each ‘o’ represents a phosphodiester internucleoside linkage. Each cytosine is a 5-methyl cytosine. Each nucleobase in the modified oligonucleotide listed in Table 9 below is complementary to SEQ ID NO: 1 (GENBANK Accession No. NT_006713.14 truncated from nucleotides 19939708 to 19967777), unless specifically stated otherwise. Non-complementary nucleobases are specified in the Nucleobase Sequence column in underlined, bold, italicized font. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

TABLE 9
Modified oligonucleotides with mixed PS/PO internucleoside linkages

| | | | | SEQ | SEQ | |
|---|---|---|---|---|---|---|
| | | | | ID | ID | |
| | | | Internucleoside | No: 1 | No: 1 | SEQ |
| Compound | Nucleobase Sequence | Sugar Motif | Linkage Motif | Start | Stop | ID |
| Number | (5′ to 3′) | (5′ to 3′) | (5′ to 3′) | Site | Site | No. |
| 1332250 | TCACTTTCATAATGCTGGA | nnnnnnnnnnnnnnnnnnd | ssssssssssssssosso | 27062 | 27079 | 37 |
| 1332252 | TCACTTTCATAATGCTGGC | nnnnnnnnnnnnnnnnnnd | ssssssssssssssosso | 27061 | 27079 | 30 |
| 1332253 | TCACTTTCATAATGCTGGA | nnnnnnnnnnnnnnnnnne | ssssssssssssssosso | 27062 | 27079 | 37 |
| 1332254 | TCACTTTCATAATGCTGGC | nnnnnnnnnnnnnnnnnne | ssssssssssssssosso | 27061 | 27079 | 30 |
| 1332259 | TCACTTTCATAATGCTGGA | eeeeeeeeeeeeeeeeeed | ssssssssssssssosso | 27062 | 27079 | 37 |
| 1332260 | TCACTTTCATAATGCTGGC | eeeeeeeeeeeeeeeeeed | ssssssssssssssosso | 27061 | 27079 | 30 |
| 1332261 | TCACTTTCATAATGCTGGA | eeeeeeeeeeeeeeeeeee | ssssssssssssssosso | 27062 | 27079 | 37 |
| 1332262 | TCACTTTCATAATGCTGGC | eeeeeeeeeeeeeeeeeee | ssssssssssssssosso | 27061 | 27079 | 30 |

### Table 10

The modified oligonucleotides in Table 10 below are each 19 nucleosides in length. Each nucleoside comprises a 2′-MOE sugar moiety or a 2′-NMA sugar moiety. The sugar motif for each modified oligonucleotide is provided in the Sugar Motif column, wherein each ‘e’ represents a 2′-MOE sugar moiety, and each ‘n’ represents a 2′-NMA sugar moiety. Each internucleoside linkage is either a phosphorothioate internucleoside linkage or a phosphodiester internucleoside linkage. The internucleoside linkage motif for each modified oligonucleotide, provided in the Internucleoside Linkage Motif column, is (from 5′ to 3′): osssssssssssssssss; wherein each ‘s’ represents a phosphorothioate internucleoside linkage, and each ‘o’ represents a phosphodiester internucleoside linkage. Each cytosine is a 5-methyl cytosine.

Each nucleobase in the modified oligonucleotide listed in Table 10 below is complementary to SEQ ID NO: 1 (GENBANK Accession No. NT_006713.14 truncated from nucleotides 19939708 to 19967777), unless specifically stated otherwise. Non-complementary nucleobases are specified in the Nucleobase Sequence column in underlined, bold, italicized font. “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

TABLE 10
Modified oligonucleotides with mixed PS/PO internucleoside linkages

| | | | | SEQ | SEQ | |
|---|---|---|---|---|---|---|
| | | | | ID | ID | |
| | | | Internucleoside | No: 1 | No: 1 | SEQ |
| Compound | Nucleobase Sequence | Sugar Motif | Linkage Motif | Start | Stop | ID |
| Number | (5′ to 3′) | (5′ to 3′) | (5′ to 3′) | Site | Site | No. |
| 1287742 | CTCACTTTCATAATGCTGG | ennnnnnnnnnnnnnnnnn | osssssssssssssssss | 27062 | 27079 | 47 |
| 1287743 | TTCACTTTCATAATGCTGG | ennnnnnnnnnnnnnnnnn | osssssssssssssssss | 27062 | 27080 | 33 |
| 1287744 | ATCACTTTCATAATGCTGG | ennnnnnnnnnnnnnnnnn | osssssssssssssssss | 27062 | 27079 | 48 |
| 1287714 | CTCACTTTCATAATGCTGG | eeeeeeeeeeeeeeeeeee | osssssssssssssssss | 27062 | 27079 | 47 |
| 1287716 | ATCACTTTCATAATGCTGG | eeeeeeeeeeeeeeeeeee | osssssssssssssssss | 27062 | 27079 | 48 |
| 1287715 | TTCACTTTCATAATGCTGG | eeeeeeeeeeeeeeeeeee | osssssssssssssssss | 27062 | 27080 | 33 |

### Table 11

The modified oligonucleotides in Table 11 below are each 20 nucleosides in length. Each nucleoside comprises a 2′-MOE sugar moiety or a 2′-NMA sugar moiety. The sugar motif for each modified oligonucleotide is provided in the Sugar Motif column, wherein each ‘e’ represents a 2′-MOE sugar moiety, and each ‘n’ represents a 2′-NMA sugar moiety. Each internucleoside linkage is either a phosphorothioate internucleoside linkage or a phosphodiester internucleoside linkage. The internucleoside linkage motif for each modified oligonucleotide, provided in the Internucleoside Linkage Motif column, is (from 5′ to 3′): ossssssssssssssssso; wherein each ‘s’ represents a phosphorothioate internucleoside linkage, and each ‘o’ represents a phosphodiester internucleoside linkage. Each cytosine is a 5-methyl cytosine.

Each modified oligonucleotide listed in Table 11 below is 100% complementary to SEQ ID NO: 1 (GENBANK Accession No. NT_006713.14 truncated from nucleotides 19939708 to 19967777). “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

TABLE 11
Modified oligonucleotides with mixed PS/PO internucleoside linkages

| | | | | SEQ | SEQ | |
|---|---|---|---|---|---|---|
| | | | Internucleoside | ID No: | ID No: | SEQ |
| Compound | Nucleobase Sequence | Sugar Motif | Linkage Motif | 1 Start | 1 Stop | ID |
| Number | (5′ to 3′) | (5′ to 3′) | (5′ to 3′) | Site | Site | No. |
| 1287745 | TTCACTTTCATAATGCTGGC | ennnnnnnnnnnnnnnnnne | ossssssssssssssssso | 27061 | 27080 | 22 |
| 1287717 | TTCACTTTCATAATGCTGGC | eeeeeeeeeeeeeeeeeeee | ossssssssssssssssso | 27061 | 27080 | 22 |

### Example 2: Activity of Modified Oligonucleotides Complementary to Human SMN2 in Transgenic Mice, Single Dose (35 μg)

Activity of selected modified oligonucleotides described above was tested in human SMN2 transgenic mice. Taiwan strain of SMA Type III mice were obtained from The Jackson Laboratory (Bar Harbor, Maine). These mice lack mouse SMN and are homozygous for human SMN2 (mSMN −/−; hSMN2 +/+; FVB.Cg-Tg(SMN2)₂HungSMN1tm1Hung/J, stock number 005058; Bar Harbor, Maine), or are heterozygous for mouse SMN and heterozygous for human SMN2(mSMN +/−; hSMN2 +/−; FVB.Cg-Tg(SMN2)2HungSMN1tm1Hung/J) obtained by breeding HOM/HOM (stock #00005058) to FVB/NJ (Stock #001800).

### Treatment

Homozygous or heterozygous transgenic mice were divided into groups of 4 mice each. Each mouse received a single ICV bolus of 35 μg of modified oligonucleotide. Comparator Compound Nos. 387954, 396442, and 396443 were also tested in this assay. A group of 4 mice received PBS as a negative control.

### RNA Analysis

Two weeks post treatment, mice were sacrificed and RNA was extracted from cortical brain tissue and spinal cord for real-time qPCR analysis of SMN2 RNA. Primer probe set hSMN2vd #4_LTS00216_MGB (forward sequence: GCTGATGCTTTGGGAAGTATGTTA (SEQ ID NO: 11); reverse sequence CACCTTCCTTCTTTTTGATTTTGTC, designated herein as SEQ ID NO: 12; probe sequence TACATGAGTGGCTATCATACT (SEQ ID NO: 13)) was used to determine the amount of SMN2 RNA including exon 7 (exon 7⁺). Primer probe set hSMN2_Sumner68_PPS50481 (forward sequence: CATGGTACATGAGTGGCTATCATACTG (SEQ ID NO: 14); reverse sequence: TGGTGTCATTTAGTGCTGCTCTATG (SEQ ID NO: 15); probe sequence CCAGCATTTCCATATAATAGC (SEQ ID NO: 16) was used to determine the amount of SMN2 RNA excluding exon 7 (exon 7⁻). Total SMN2 RNA levels were measured using primer probe set hSMN2_LTS00935 (forward sequence: CAGGAGGATTCCGTGCTGTT (SEQ ID NO: 17); reverse sequence CAGTGCTGTATCATCCCAAATGTC, (SEQ ID NO: 18); probe sequence: ACAGGCCAGAGCGAT (SEQ ID NO: 19)).

Results are presented as fold change in RNA levels relative to PBS control, normalized to total SMN2 levels. Each of Tables 12-18 represents a different experiment.

TABLE 12
Effect of modified oligonucleotides on human SMN2
RNA splicing in homozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1 | 1 | 1 | 1 |
| 396442 | 35 | 3.3 | 0.3 | 3.4 | 0.3 |
| 396443 | 35 | 3.0 | 0.5 | 2.3 | 0.5 |
| 524403 | 35 | 3.3 | 0.4 | 2.5 | 0.5 |
| 1210339 | 35 | 2.5 | 0.5 | 3.0 | 0.3 |
| 1210340 | 35 | 2.1 | 0.6 | 2.6 | 0.4 |
| 1210341 | 35 | 1.8 | 0.7 | 2.0 | 0.6 |
| 1210342 | 35 | 2.5 | 0.5 | 2.9 | 0.3 |
| 1210343 | 35 | 3.0 | 0.4 | 2.4 | 0.5 |
| 1212817 | 35 | 2.4 | 0.6 | 2.2 | 0.6 |
| 1212818 | 35 | 2.4 | 0.5 | 2.1 | 0.6 |
| 1212823 | 35 | 2.0 | 0.6 | 2.0 | 0.6 |
| 1212824 | 35 | 2.1 | 0.6 | 2.1 | 0.6 |
| 1212825 | 35 | 2.9 | 0.4 | 2.5 | 0.5 |
| 1212826 | 35 | 2.5 | 0.6 | 2.2 | 0.7 |
| 1212827 | 35 | 2.5 | 0.6 | 2.6 | 0.5 |
| 1212828 | 35 | 2.9 | 0.5 | 2.4 | 0.6 |
| 1212830 | 35 | 2.8 | 0.7 | 2.1 | 0.8 |
| 1212831 | 35 | 2.5 | 0.7 | 2.3 | 0.7 |
| 1212832 | 35 | 2.9 | 0.6 | 2.9 | 0.5 |
| 1212833 | 35 | 2.4 | 0.7 | 2.7 | 0.5 |
| 1212837 | 35 | 2.5 | 0.6 | 2.7 | 0.5 |
| 1212838 | 35 | 2.1 | 0.7 | 2.5 | 0.6 |
| 1212844 | 35 | 2.6 | 0.6 | 2.4 | 0.7 |
| 1212845 | 35 | 2.3 | 0.7 | 2.5 | 0.7 |
| 1212846 | 35 | 2.8 | 0.6 | 2.6 | 0.6 |
| 1212849 | 35 | 2.1 | 0.7 | 2.3 | 0.6 |
| 1212850 | 35 | 1.8 | 0.8 | 2.2 | 0.7 |
| 1212855 | 35 | 2.0 | 0.7 | 2.1 | 0.8 |

TABLE 13
Effect of modified oligonucleotides on human SMN2
RNA splicing in homozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1.0 | 1.0 | 1.0 | 1.0 |
| 396443 | 35 | 2.7 | 0.3 | 1.9 | 0.5 |
| 1210342 | 35 | 2.4 | 0.5 | 2.5 | 0.4 |
| 1212961 | 35 | 1.8 | 0.7 | 1.7 | 0.6 |
| 1212962 | 35 | 2.0 | 0.6 | 1.9 | 0.5 |
| 1212963 | 35 | 2.3 | 0.5 | 2.5 | 0.3 |
| 1212966 | 35 | 1.6 | 0.8 | 2.0 | 0.5 |
| 1212967 | 35 | 1.9 | 0.6 | 1.9 | 0.4 |
| 1212971 | 35 | 1.6 | 0.5 | 2.0 | 0.4 |
| 1212972 | 35 | 1.8 | 0.6 | 2.2 | 0.5 |
| 1212977 | 35 | 2.1 | 0.5 | 2.2 | 0.4 |
| 1212978 | 35 | 2.1 | 0.6 | 2.2 | 0.4 |
| 1212979 | 35 | 2.1 | 0.5 | 2.6 | 0.3 |
| 1212982 | 35 | 2.0 | 0.7 | 1.8 | 0.6 |
| 1212983 | 35 | 1.9 | 0.6 | 1.7 | 0.5 |
| 1212984 | 35 | 1.9 | 0.6 | 1.9 | 0.5 |
| 1212987 | 35 | 2.4 | 0.4 | 2.5 | 0.4 |
| 1212988 | 35 | 1.8 | 0.7 | 1.8 | 0.5 |
| 1212995 | 35 | 2.5 | 0.5 | 2.5 | 0.4 |
| 1212998 | 35 | 1.8 | 0.6 | 1.8 | 0.7 |
| 1212999 | 35 | 2.0 | 0.6 | 2.0 | 0.5 |
| 1213003 | 35 | 1.9 | 0.7 | 2.3 | 0.5 |
| 1213004 | 35 | 1.8 | 0.7 | 2.3 | 0.6 |

TABLE 14
Effect of modified oligonucleotides on human SMN2
RNA splicing in homozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1.0 | 1.0 | 1.0 | 1.0 |
| 396443 | 35 | 2.6 | 0.5 | 3.1 | 0.5 |
| 1212964 | 35 | 2.5 | 0.6 | 3.6 | 0.4 |
| 1212965 | 35 | 2.9 | 0.5 | 3.3 | 0.4 |
| 1212968 | 35 | 2.2 | 0.6 | 2.3 | 0.6 |
| 1212973 | 35 | 2.6 | 0.5 | 3.2 | 0.4 |
| 1212974 | 35 | 2.3 | 0.6 | 2.8 | 0.5 |
| 1212975 | 35 | 2.9 | 0.3 | 3.1 | 0.4 |
| 1212976 | 35 | 2.5 | 0.5 | 2.8 | 0.5 |
| 1212980 | 35 | 2.6 | 0.5 | 3.2 | 0.4 |
| 1212981 | 35 | 2.9 | 0.4 | 3.6 | 0.3 |
| 1212985 | 35 | 2.4 | 0.6 | 2.9 | 0.5 |
| 1212986 | 35 | 2.8 | 0.4 | 3.3 | 0.4 |
| 1212989 | 35 | 3.3 | 0.3 | 3.6 | 0.2 |
| 1212990 | 35 | 1.8 | 0.8 | 2.1 | 0.7 |
| 1212991 | 35 | 3.2 | 0.3 | 3.8 | 0.3 |
| 1212992 | 35 | 2.4 | 0.5 | 2.2 | 0.6 |
| 1212996 | 35 | 2.2 | 0.6 | 3.2 | 0.5 |
| 1212997 | 35 | 2.9 | 0.4 | 3.9 | 0.4 |
| 1213001 | 35 | 2.1 | 0.5 | 2.8 | 0.6 |
| 1213002 | 35 | 2.0 | 0.6 | 2.9 | 0.6 |
| 1213005 | 35 | 2.8 | 0.5 | 3.2 | 0.3 |
| 1213006 | 35 | 1.9 | 0.9 | 2.0 | 0.8 |
| 1213007 | 35 | 3.3 | 0.2 | 2.9 | 0.5 |
| 1213008 | 35 | 2.3 | 0.7 | 2.2 | 0.7 |

TABLE 15
Effect of modified oligonucleotides on human SMN2
RNA splicing in heterozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1.0 | 1.0 | 1.0 | 1.0 |
| 387954 | 35 | 2.3 | 0.6 | 2.2 | 0.5 |
| 396443 | 35 | 2.5 | 0.5 | 2.4 | 0.5 |
| 1287048 | 35 | 2.2 | 0.5 | 2.2 | 0.5 |
| 1287049 | 35 | 2.3 | 0.6 | 2.5 | 0.4 |
| 1287061 | 35 | 2.4 | 0.5 | 2.2 | 0.4 |
| 1287062 | 35 | 3.0 | 0.3 | 2.3 | 0.4 |
| 1287050 | 35 | 2.8 | 0.5 | 2.3 | 0.4 |
| 1287054 | 35 | 2.2 | 0.5 | 2.3 | 0.4 |
| 1287063 | 35 | 1.8 | 0.7 | 1.7 | 0.6 |
| 1287064 | 35 | 2.6 | 0.3 | 2.4 | 0.4 |
| 1287065 | 35 | 2.5 | 0.4 | 2.3 | 0.4 |
| 1287066 | 35 | 2.2 | 0.5 | 2 | 0.5 |
| 1287075 | 35 | 2.3 | 0.6 | 1.8 | 0.7 |
| 1287076 | 35 | 2.6 | 0.4 | 1.9 | 0.6 |
| 1287067 | 35 | 2.7 | 0.4 | 1.9 | 0.6 |
| 1287070 | 35 | 2.5 | 0.5 | 1.8 | 0.7 |
| 1287071 | 35 | 2.6 | 0.4 | 1.8 | 0.7 |
| 1287074 | 35 | 2.6 | 0.5 | 2 | 0.6 |
| 1287109 | 35 | 2.7 | 0.6 | 2.4 | 0.5 |
| 1287110 | 35 | 2.6 | 0.5 | 2.3 | 0.5 |
| 1287701 | 35 | 2.6 | 0.6 | 2.8 | 0.3 |
| 1287702 | 35 | 3 | 0.5 | 2.8 | 0.4 |
| 1287703 | 35 | 2.3 | 0.6 | 2.4 | 0.4 |
| 1287704 | 35 | 2.7 | 0.5 | 2.3 | 0.4 |
| 1287717 | 35 | 3.3 | 0.3 | 2.4 | 0.6 |

TABLE 16
Effect of modified oligonucleotides on human SMN2
RNA splicing in heterozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1 | 1 | 1 | 1 |
| 396442 | 35 | 2.5 | 0.6 | 3.2 | 0.3 |
| 396443 | 35 | 3 | 0.5 | 2.8 | 0.5 |
| 1263783 | 35 | 3 | 0.3 | 2.6 | 0.5 |
| 1263785 | 35 | 3.1 | 0.4 | 2.9 | 0.5 |
| 1263787 | 35 | 2.4 | 0.6 | 2.9 | 0.4 |
| 1263789 | 35 | 3.8 | 0.2 | 2.6 | 0.5 |
| 1263800 | 35 | 3.6 | 0.2 | 2.6 | 0.5 |
| 1263802 | 35 | 3.4 | 0.3 | 2.9 | 0.4 |
| 1263806 | 35 | 3.5 | 0.2 | 2.7 | 0.5 |
| 1263808 | 35 | 3.2 | 0.4 | 2.7 | 0.5 |
| 1263810 | 35 | 2.8 | 0.5 | 2.4 | 0.5 |

TABLE 17
Effect of modified oligonucleotides on human SMN2
RNA splicing in heterozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1 | 1 | 1 | 1 |
| 396443 | 35 | 2.5 | 0.6 | 2.9 | 0.4 |
| 1364784 | 35 | 2.3 | 0.7 | 2.8 | 0.5 |
| 1364783 | 35 | 2.9 | 0.5 | 2.4 | 0.5 |
| 1364777 | 35 | 2.7 | 0.6 | 2.3 | 0.5 |
| 1364782 | 35 | 2.7 | 0.6 | 2.6 | 0.5 |

TABLE 18
Effect of modified oligonucleotides on human SMN2
RNA splicing in heterozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1 | 1 | 1 | 1 |
| 396443 | 35 | 2 | 0.7 | 2.7 | 0.5 |
| 1318748 | 35 | 2.1 | 0.7 | 2.5 | 0.6 |
| 1318782 | 35 | 2.2 | 0.8 | 2.5 | 0.6 |
| 1332262 | 35 | 3.3 | 0.4 | 2.9 | 0.5 |
| 1332258 | 35 | 2.4 | 0.7 | 2.3 | 0.6 |

### Example 3: Activity of Modified Oligonucleotides Complementary to Human SMN2 in Transgenic Mice, Single Dose (15 μg)

Activity of selected modified oligonucleotides described above was tested in human SMN2 transgenic mice essentially as described above in Example 2. Comparator Compound Nos. 396443 and 819735 were also tested in this assay. The transgenic mice were divided into groups of 4 mice each. Each mouse received a single ICV bolus of 15 μg of modified oligonucleotide. A group of 4 mice received PBS as a negative control. Two weeks post treatment, mice were sacrificed and RNA was extracted from cortical brain tissue and spinal cord for real-time qPCR analysis of SMN2 RNA. Results are presented as fold change in RNA levels relative to PBS control, normalized to total SMN2 levels. Each of Tables 19-23 represents a different experiment.

TABLE 19
Effect of modified oligonucleotides on human SMN2
RNA splicing in homozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1.0 | 1.0 | 1.0 | 1.0 |
| 819735 | 15 | 2.4 | 0.4 | 3.3 | 0.3 |
| 1212869 | 15 | 2.4 | 0.4 | 3.2 | 0.4 |
| 1212870 | 15 | 2.1 | 0.5 | 2.8 | 0.4 |
| 1212873 | 15 | 2.2 | 0.4 | 2.0 | 0.6 |
| 1212874 | 15 | 2.1 | 0.5 | 2.4 | 0.6 |
| 1212875 | 15 | 2.1 | 0.5 | 2.3 | 0.5 |
| 1212880 | 15 | 1.7 | 0.6 | 2.0 | 0.6 |
| 1212881 | 15 | 1.8 | 0.6 | 2.3 | 0.6 |
| 1212885 | 15 | 2.3 | 0.4 | 2.4 | 0.5 |
| 1212887 | 15 | 2.0 | 0.5 | 2.2 | 0.5 |
| 1212931 | 15 | 2.9 | 0.2 | 2.9 | 0.3 |
| 1212936 | 15 | 2.9 | 0.3 | 3.3 | 0.3 |
| 1212941 | 15 | 3.0 | 0.1 | 3.4 | 0.2 |

TABLE 20
Effect of modified oligonucleotides on human SMN2
RNA splicing in heterozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1.0 | 1.0 | 1.0 | 1.0 |
| 396443 | 15 | 2.2 | 0.5 | 2.2 | 0.7 |
| 819735 | 15 | 2.7 | 0.5 | 3.1 | 0.5 |
| 1287122 | 15 | 2.9 | 0.5 | 2.3 | 0.6 |
| 1287123 | 15 | 3.0 | 0.4 | 3.0 | 0.4 |
| 1287124 | 15 | 3.0 | 0.4 | 3.2 | 0.3 |
| 1287125 | 15 | 3.0 | 0.4 | 3.0 | 0.4 |
| 1287126 | 15 | 2.8 | 0.4 | 2.8 | 0.4 |
| 1287127 | 15 | 2.7 | 0.5 | 3.0 | 0.5 |
| 1287128 | 15 | 2.6 | 0.5 | 3.2 | 0.5 |
| 1287129 | 15 | 2.9 | 0.4 | 2.9 | 0.5 |
| 1287130 | 15 | 3.7 | 0.1 | 3.1 | 0.5 |
| 1287131 | 15 | 2.2 | 0.6 | 2.7 | 0.4 |
| 1287132 | 15 | 3.2 | 0.3 | 2.2 | 0.6 |
| 1287133 | 15 | 2.9 | 0.4 | 2.8 | 0.4 |
| 1287728 | 15 | 2.8 | 0.6 | 3.4 | 0.3 |
| 1287729 | 15 | 3.1 | 0.4 | 3.0 | 0.3 |
| 1287730 | 15 | 3.1 | 0.3 | 2.7 | 0.4 |
| 1287731 | 15 | 3.3 | 0.3 | 2.8 | 0.5 |
| 1287735 | 15 | 2.9 | 0.5 | 2.6 | 0.5 |
| 1287738 | 15 | 3.7 | 0.2 | 3.2 | 0.3 |
| 1287739 | 15 | 3.3 | 0.4 | 3.2 | 0.4 |
| 1287743 | 15 | 3.6 | 0.4 | 3.8 | 0.4 |
| 1287745 | 15 | 3.1 | 0.5 | 3.8 | 0.5 |

TABLE 21
Effect of modified oligonucleotides on human SMN2
RNA splicing in heterozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1 | 1 | 1 | 1.0 |
| 396443 | 15 | 1.9 | 0.6 | 1.7 | 0.7 |
| 819735 | 15 | 2.3 | 0.5 | 1.9 | 0.6 |

TABLE 22
Effect of modified oligonucleotides on human SMN2
RNA splicing in heterozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1 | 1 | 1 | 1 |
| 1364781 | 15 | 2.5 | 0.6 | 2.7 | 0.4 |
| 1364780 | 15 | 2.8 | 0.5 | 2.6 | 0.5 |
| 1364779 | 15 | 2.7 | 0.5 | 2.6 | 0.5 |
| 1364778 | 15 | 3 | 0.5 | 2.7 | 0.4 |

TABLE 23
Effect of modified oligonucleotides on human SMN2
RNA splicing in heterozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1 | 1 | 1 | 1 |
| 819735 | 15 | 2.8 | 0.5 | 2.4 | 0.6 |
| 1332265 | 15 | 2.1 | 0.7 | 2.6 | 0.6 |
| 1332269 | 15 | 2.5 | 0.6 | 2.7 | 0.5 |
| 1332268 | 15 | 2.9 | 0.5 | 2.3 | 0.6 |
| 1318756 | 15 | 2.2 | 0.7 | 2.4 | 0.6 |
| 1333508 | 15 | 2 | 0.6 | 2.2 | 0.6 |
| 1332251 | 15 | 2.9 | 0.5 | 1.9 | 0.7 |
| 1332249 | 15 | 2.3 | 0.7 | 2.3 | 0.7 |

### Example 4: Activity of Modified Oligonucleotides Complementary to Human SMN2 in Transgenic Mice, Single Dose (70 μg)

Activity of modified oligonucleotides was tested in human SMN2 transgenic mice essentially as described above in Example 2. The transgenic mice were divided into groups of 4 mice each. Each mouse received a single ICV bolus of 70 μg modified oligonucleotide. A group of 4 mice received PBS as a negative control. Two weeks post treatment, mice were sacrificed and RNA was extracted from cortical brain tissue and spinal cord for real-time qPCR analysis of SMN2 RNA. Results are presented as fold change in RNA levels relative to PBS control, normalized to total SMN2 levels.

TABLE 24
Effect of modified oligonucleotides on human SMN2
RNA splicing in homozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1 | 1 | 1 | 1 |
| U12969 | 70 | 2.5 | 0.4 | 2.4 | 0.3 |
| U12970 | 70 | 2.7 | 0.3 | 2.6 | 0.3 |

### Example 5: Activity of Modified Oligonucleotides Complementary to Human SMN2 in Transgenic Mice, Multiple Dose

Activity of selected modified oligonucleotides described above was tested in human SMN2 transgenic mice essentially as described above in Example 2. Comparator Compound No. 396443 was also tested in this assay. The transgenic mice were divided into groups of 4 mice each. Each mouse received a single ICV bolus of modified oligonucleotide at multiple doses as indicated in the tables below. A group of 4 mice received PBS as a negative control. Two weeks post treatment, mice were sacrificed and RNA was extracted from coronal brain and spinal cord for real-time qPCR analysis of SMN2 RNA. Results are presented as fold change in RNA levels relative to PBS control, normalized to total SMN2 levels. ED₅₀ for exon inclusion (exon 7⁺) was calculated in GraphPad Prism 7 using nonlinear regression, 4-parameter dose response curve [Y=Bottom+(Top−Bottom)/(1+(10\hat{ } log EC50/X)\hat{ }HillSlope)].

TABLE 25
Effect of modified oligonucleotides on human
SMN2 RNA splicing in homozygous transgenic mice

| | Dose | CORONAL BRAIN | | ED50 | SPINAL CORD | | ED50 |
|---|---|---|---|---|---|---|---|
| Compound No. | (μg) | exon 7⁺ | exon 7⁻ | (μg) | exon 7⁺ | exon 7⁻ | (μg) |
| PBS | — | 1.0 | 1.0 | | 1.0 | 1.0 | |
| 396443 | 3 | 1.4 | 0.9 | 32.5 | 1.3 | 0.9 | 22.1 |
| | 10 | 1.8 | 0.8 | | 2.0 | 0.7 | |
| | 30 | 2.6 | 0.5 | | 2.6 | 0.4 | |
| | 100 | 3.5 | 0.4 | | 3.2 | 0.3 | |
| | 300 | 4.2 | 0.1 | | 3.6 | 0.2 | |
| 1263789 | 3 | 1.5 | 0.9 | 38.3 | 1.5 | 0.8 | 13.3 |
| | 10 | 2.0 | 0.7 | | 2.2 | 0.6 | |
| | 30 | 2.3 | 0.6 | | 3.0 | 0.4 | |
| | 100 | 3.4 | 0.3 | | 3.4 | 0.3 | |
| | 300 | 3.9 | 0.1 | | 3.7 | 0.2 | |
| 1287717 | 3 | 1.3 | 0.8 | 38.7 | 1.3 | 0.9 | 20.5 |
| | 10 | 1.8 | 0.7 | | 1.9 | 0.7 | |
| | 30 | 2.4 | 0.7 | | 2.7 | 0.5 | |
| | 100 | 3.5 | 0.4 | | 3.3 | 0.3 | |
| | 300 | 4.1 | 0.1 | | 3.8 | 0.2 | |
| 1358996 | 3 | 1.6 | 0.9 | 16.6 | 1.7 | 0.8 | 7.4 |
| | 10 | 2.5 | 0.6 | | 2.6 | 0.5 | |
| | 30 | 3.0 | 0.4 | | 3.5 | 0.2 | |
| | 100 | 4.0 | 0.2 | | 3.6 | 0.2 | |
| | 300 | 4.0 | 0.1 | | 3.9 | 0.1 | |
| 1287745 | 3 | 1.5 | 0.8 | 22.8 | 1.7 | 0.7 | 8.8 |
| | 10 | 2.1 | 0.6 | | 2.4 | 0.5 | |
| | 30 | 3.0 | 0.3 | | 3.3 | 0.3 | |
| | 100 | 3.6 | 0.1 | | 3.5 | 0.2 | |
| | 300 | 4.2 | 0.1 | | 3.8 | 0.1 | |

### Example 6: Tolerability of Modified Oligonucleotides Complementary to SMN2 in Wild-Type Mice, 3 Hour Study

Modified oligonucleotides described above were tested in wild-type female C57/B16 mice to assess tolerability. Wild-type female C57/B16 mice each received a single ICV dose of 700 ag of modified oligonucleotide listed in the tables below. Comparator Compound No. 396443 was also tested in this assay with a dose of 350 μg. Comparator Compound Nos. 387954, 396442, 443305, and 819735 were also tested in this assay with a dose of 700 μg. Each treatment group consisted of 4 mice. A group of 4 mice received PBS as a negative control for each experiment (identified in separate tables below). At 3 hours post-injection, mice were evaluated according to seven different criteria. The criteria are (1) the mouse was bright, alert, and responsive; (2) the mouse was standing or hunched without stimuli; (3) the mouse showed any movement without stimuli; (4) the mouse demonstrated forward movement after it was lifted: (5) the mouse demonstrated any movement after it was lifted: (6) the mouse responded to tail pinching: (7) regular breathing. For each of the 7 criteria, a mouse was given a subscore of 0 if it met the criteria and 1 if it did not (the functional observational battery score or FOB). After all 7 criteria were evaluated, the scores were summed and averaged within each treatment group. The results are presented in the tables below. Each of Tables 26-49 represents a different experiment.

TABLE 26
Tolerability dose scores
in mice at 350 μg

| Compound | 3 hr |
|---|---|
| Number | FOB |
| PBS | 0 |
| 396443 | 4.0 |

TABLE 27
Tolerability dose scores
in mice at 700 μg

| Compound | 3 hr |
|---|---|
| Number | FOB |
| PBS | 0.00 |
| 443305 | 4.75 |

TABLE 28
Tolerability scores in
mice at 700 μg dose

| Compound | 3 hr |
|---|---|
| Number | FOB |
| PBS | 0 |
| 396442 | 2.5 |
| 524403 | 3.25 |
| 1210339 | 1.25 |
| 1210340 | 2.25 |
| 1210341 | 3.75 |
| 1210342 | 0 |
| 1210343 | 0 |
| 1212817 | 0 |
| 1212818 | 0 |
| 1212819 | 0 |
| 1212820 | 0 |
| 1212821 | 0 |
| 1212822 | 0 |
| 1212823 | 0 |
| 1212824 | 0 |
| 1212825 | 1 |
| 1212826 | 0 |
| 1212827 | 0 |
| 1212828 | 0 |
| 1212829 | 0 |
| 1212830 | 0 |
| 1212831 | 0 |

TABLE 29
Tolerability scores in
mice at 700 μg dose

| Compound | 3 hr |
|---|---|
| Number | FOB |
| PBS | 0.00 |
| 396442 | 2.50 |
| 1210340 | 3.50 |
| 1212850 | 0.50 |
| 1212851 | 0.75 |
| 1212852 | 0.00 |
| 1212853 | 0.00 |
| 1212854 | 0.25 |
| 1212855 | 0.25 |
| 1212856 | 0.00 |
| 1212857 | 0.00 |
| 1212858 | 0.00 |
| 1212859 | 0.00 |
| 1212860 | 0.75 |
| 1212861 | 1.00 |
| 1212863 | 2.00 |
| 1212864 | 0.00 |
| 1212866 | 0.75 |
| 1212867 | 0.00 |
| 1212868 | 0.00 |

TABLE 30
Tolerability scores in
mice at 700 μg dose

| Compound | 3 hr |
|---|---|
| Number | FOB |
| PBS | 0.00 |
| 396442 | 3.25 |
| 1212961 | 0.00 |
| 1212963 | 1.00 |
| 1212964 | 2.00 |
| 1212965 | 1.25 |
| 1212966 | 1.25 |
| 1212968 | 0.00 |
| 1212971 | 1.00 |
| 1212972 | 3.25 |
| 1212973 | 0.50 |
| 1212974 | 2.00 |
| 1212975 | 0.50 |
| 1212976 | 1.75 |

TABLE 31
Tolerability scores in
mice at 700 μg dose

| Compound | 3 hr |
|---|---|
| Number | FOB |
| PBS | 0.00 |
| 1212977 | 0.75 |
| 1212978 | 0.00 |
| 1212979 | 1.75 |
| 1212980 | 1.50 |
| 1212981 | 0.00 |
| 1212982 | 0.50 |
| 1212983 | 0.75 |
| 1212984 | 2.75 |
| 1212985 | 0.00 |
| 1212986 | 1.00 |
| 1212987 | 1.75 |
| 1212988 | 4.50 |
| 1212989 | 1.75 |
| 1212990 | 4.50 |
| 1212991 | 1.25 |
| 1212992 | 3.75 |

TABLE 32
Tolerability scores in
mice at 700 μg dose

| Compound | 3 hr |
|---|---|
| Number | FOB |
| PBS | 0.00 |
| 1212993 | 7.00 |
| 1212994 | 6.50 |
| 1212995 | 4.25 |
| 1212996 | 3.25 |
| 1212997 | 4.00 |
| 1212998 | 2.00 |
| 1212999 | 1.00 |
| 1213000 | 1.25 |
| 1213001 | 3.00 |
| 1213002 | 2.00 |
| 1213003 | 4.00 |
| 1213004 | 3.00 |
| 1213005 | 3.75 |
| 1213006 | 4.00 |
| 1213007 | 4.00 |
| 1213008 | 3.50 |

TABLE 33
Tolerability scores in
mice at 700 μg dose

| Compound | 3 hr |
|---|---|
| Number | FOB |
| PBS | 0.00 |
| 1212832 | 0.00 |
| 1212833 | 0.00 |
| 1212834 | 0.00 |
| 1212835 | 0.00 |
| 1212836 | 0.00 |
| 1212837 | 0.00 |
| 1212838 | 0.00 |
| 1212839 | 0.00 |
| 1212840 | 0.00 |
| 1212841 | 0.00 |
| 1212842 | 0.00 |
| 1212843 | 0.00 |
| 1212844 | 0.25 |
| 1212845 | 1.00 |
| 1212846 | 0.00 |
| 1212847 | 0.00 |
| 1212848 | 0.00 |
| 1212849 | 0.00 |

TABLE 34
Tolerability scores in
mice at 700 μg dose

| Compound | 3 hr |
|---|---|
| Number | FOB |
| PBS | 0.00 |
| 396442 | 1.75 |
| 1210339 | 1.00 |
| 1212865 | 1.00 |
| 1212962 | 0.00 |
| 1212967 | 0.50 |
| 1212969 | 0.50 |
| 1212970 | 1.25 |

TABLE 35
Tolerability scores in
mice at 700 μg dose

| Compound | 3 hr |
|---|---|
| Number | FOB |
| PBS | 0.00 |
| 819735 | 2.00 |
| 1212869 | 2.00 |
| 1212870 | 4.75 |
| 1212871 | 1.00 |
| 1212873 | 0.00 |
| 1212874 | 0.00 |
| 1212875 | 0.00 |
| 1212879 | 3.00 |
| 1212880 | 0.00 |
| 1212881 | 4.00 |
| 1212885 | 1.00 |
| 1212887 | 2.25 |
| 1212931 | 2.00 |
| 1212936 | 2.00 |
| 1212941 | 1.25 |

TABLE 36
Tolerability scores in
mice at 700 μg dose

| Compound | 3 hr |
|---|---|
| Number | FOB |
| PBS | 0.00 |
| 1263778 | 0.00 |
| 1263781 | 0.00 |
| 1263783 | 0.00 |
| 1263785 | 1.00 |
| 1263787 | 0.00 |
| 1263789 | 0.00 |
| 1263791 | 0.00 |
| 1263793 | 0.00 |
| 1263795 | 0.00 |
| 1263797 | 0.00 |
| 1263799 | 0.00 |
| 1263800 | 0.00 |
| 1263802 | 0.00 |
| 1263804 | 0.00 |
| 1263806 | 0.00 |
| 1263808 | 1.00 |
| 1263810 | 0.00 |
| 1263812 | 0.00 |
| 1263814 | 1.00 |
| 1263816 | 0.50 |
| 1263818 | 0.00 |
| 1263820 | 0.00 |
| 1263822 | 0.25 |
| 1263824 | 0.00 |

TABLE 37
Tolerability scores in
mice at 700 μg dose

| Compound | 3 hr |
|---|---|
| Number | FOB |
| PBS | 0.00 |
| 1263826 | 0.00 |

TABLE 38
Tolerability scores in
mice at 700 μg dose

| Compound | 3 hr |
|---|---|
| Number | FOB |
| PBS | 0.00 |
| 387954 | 4.00 |
| 1287048 | 0.00 |
| 1287049 | 0.00 |
| 1287050 | 2.00 |
| 1287051 | 3.25 |
| 1287052 | 3.50 |
| 1287053 | 2.75 |
| 1287054 | 2.00 |
| 1287055 | 3.25 |
| 1287056 | 4.00 |
| 1287057 | 3.00 |
| 1287058 | 4.00 |
| 1287059 | 4.00 |
| 1287060 | 4.00 |
| 1287061 | 4.00 |
| 1287062 | 3.50 |

TABLE 39
Tolerability scores in
mice at 700 μg dose

| Compound | 3 hr |
|---|---|
| Number | FOB |
| PBS | 0.00 |
| 1287106 | 3.50 |
| 1287107 | 4.00 |
| 1287108 | 3.75 |
| 1287109 | 3.25 |
| 1287110 | 3.00 |
| 1287111 | 4.75 |
| 1287112 | 4.00 |
| 1287113 | 3.50 |
| 1287114 | 3.25 |
| 1287115 | 3.50 |
| 1287116 | 4.00 |
| 1287117 | 4.25 |
| 1287118 | 3.00 |
| 1287119 | 3.50 |
| 1287120 | 3.75 |
| 1287121 | 2.75 |

TABLE 40
Tolerability scores in
mice at 700 μg dose

| Compound | 3 hr |
|---|---|
| Number | FOB |
| PBS | 0.00 |
| 1287063 | 0.00 |
| 1287064 | 0.00 |
| 1287065 | 1.00 |
| 1287066 | 3.75 |
| 1287067 | 1.00 |
| 1287068 | 2.50 |
| 1287069 | 2.25 |
| 1287071 | 1.00 |
| 1287072 | 3.00 |
| 1287073 | 3.75 |
| 1287074 | 1.75 |
| 1287075 | 3.50 |
| 1287076 | 2.00 |

TABLE 41
Tolerability scores in
mice at 700 μg dose

| Compound | 3 hr |
|---|---|
| Number | FOB |
| PBS | 0.00 |
| 1287070 | 2.00 |
| 1287701 | 2.50 |
| 1287702 | 3.75 |
| 1287703 | 3.75 |
| 1287705 | 4.00 |
| 1287706 | 4.00 |
| 1287707 | 4.00 |
| 1287709 | 4.75 |
| 1287710 | 4.00 |
| 1287711 | 4.75 |
| 1287712 | 4.00 |
| 1287713 | 4.00 |
| 1287714 | 3.50 |
| 1287715 | 4.00 |
| 1287716 | 4.00 |
| 1287717 | 3.25 |

TABLE 42
Tolerability scores in
mice at 700 μg dose

| Compound | 3 hr |
|---|---|
| Number | FOB |
| PBS | 0.00 |
| 1287728 | 1.00 |
| 1287729 | 1.25 |
| 1287730 | 2.00 |
| 1287731 | 2.50 |
| 1287732 | 3.00 |
| 1287733 | 3.25 |
| 1287734 | 3.00 |
| 1287735 | 0.50 |
| 1287736 | 2.50 |
| 1287737 | 4.00 |
| 1287738 | 3.00 |
| 1287739 | 2.50 |
| 1287740 | 2.75 |
| 1287741 | 3.75 |
| 1287742 | 3.00 |
| 1287743 | 2.75 |
| 1287744 | 2.25 |
| 1287745 | 1.00 |

TABLE 43
Tolerability scores in
mice at 700 μg dose

| Compound | 3 hr |
|---|---|
| Number | FOB |
| PBS | 0.00 |
| 1287122 | 0.00 |
| 1287123 | 0.00 |
| 1287124 | 3.50 |
| 1287125 | 3.00 |
| 1287126 | 3.00 |
| 1287127 | 0.00 |
| 1287128 | 0.00 |
| 1287129 | 4.00 |
| 1287130 | 2.75 |
| 1287131 | 2.50 |
| 1287132 | 2.75 |
| 1287133 | 3.25 |
| 1287704 | 3.50 |
| 1287708 | 3.50 |

TABLE 44
Tolerability scores in
mice at 700 μg dose

| Compound | 3 hr |
|---|---|
| Number | FOB |
| PBS | 0.00 |
| 1318748 | 2.00 |
| 1318765 | 4.00 |
| 1318767 | 4.25 |
| 1318770 | 3.75 |
| 1318771 | 4.50 |
| 1318772 | 4.25 |
| 1318773 | 4.25 |
| 1318774 | 3.50 |
| 1318775 | 3.75 |
| 1318776 | 3.75 |
| 1318777 | 4.00 |
| 1318778 | 4.00 |
| 1318779 | 4.00 |
| 1318780 | 4.00 |
| 1318781 | 4.00 |
| 1318782 | 1.00 |
| 1318783 | 4.00 |
| 1318784 | 2.00 |

TABLE 45
Tolerability scores in
mice at 700 μg dose

| Compound | 3 hr |
|---|---|
| Number | FOB |
| PBS | 0.00 |
| 1318757 | 4.00 |
| 1318758 | 4.25 |
| 1318759 | 3.75 |
| 1318760 | 3.75 |
| 1318761 | 4.00 |
| 1318762 | 4.00 |
| 1318763 | 4.00 |
| 1318764 | 3.75 |
| 1318766 | 3.75 |
| 1318768 | 4.00 |
| 1318769 | 4.00 |

TABLE 46
Tolerability scores in
mice at 700 μg dose

| Compound | 3 hr |
|---|---|
| Number | FOB |
| PBS | 0.00 |
| 1318749 | 4.25 |
| 1318750 | 2.25 |
| 1318751 | 4.00 |
| 1318752 | 3.75 |
| 1318753 | 2.25 |
| 1318754 | 3.00 |
| 1318755 | 3.75 |
| 1318756 | 0.00 |

TABLE 47
Tolerability scores in
mice at 700 μg dose

| Compound | 3 hr |
|---|---|
| Number | FOB |
| PBS | 0.00 |
| 1332247 | 1.75 |
| 1332248 | 0.25 |
| 1332249 | 0.00 |
| 1332250 | 3.75 |
| 1332251 | 0.00 |
| 1332252 | 3.00 |
| 1332263 | 2.00 |
| 1332265 | 1.50 |
| 1332266 | 1.00 |
| 1332267 | 3.75 |
| 1332268 | 2.75 |
| 1332269 | 1.25 |
| 1332270 | 2.25 |
| 1332271 | 2.50 |
| 1333508 | 0.00 |

TABLE 48
Tolerability scores in
mice at 700 μg dose

| Compound | 3 hr |
|---|---|
| Number | FOB |
| PBS | 0.00 |
| 1332255 | 1.00 |
| 1332256 | 2.00 |
| 1332257 | 1.25 |
| 1332258 | 1.25 |
| 1332259 | 2.25 |
| 1332260 | 2.25 |
| 1332261 | 2.50 |
| 1332262 | 2.00 |

TABLE 49
Tolerability scores in
mice at 700 μg dose

| Compound | 3 hr |
|---|---|
| Number | FOB |
| PBS | 0.00 |
| 1358996 | 0.00 |
| 1364777 | 2.00 |
| 1364778 | 3.00 |
| 1364779 | 3.50 |
| 1364780 | 3.50 |
| 1364781 | 5.25 |
| 1364782 | 2.50 |
| 1364783 | 3.50 |
| 1364784 | 3.50 |

### Example 7: Tolerability of Modified Oligonucleotides Complementary to Human SMN2 in Rats, Long-Term Assessment

In separate studies run under the same conditions, selected modified oligonucleotides described above were tested in Sprague Dawley rats to assess long-term tolerability. Comparator Compound Nos. 396442 and 819735 were also tested in this assay. Sprague Dawley rats each received a single intrathecal (IT) delivered dose of 3 mg of oligonucleotide or PBS. Beginning 1 week post-treatment, each animal was weighed and evaluated weekly by a trained observer for adverse events. Adverse events were defined as neurological dysfunction not typical in PBS-treated control animals, including, but not limited to: abnormal limb splay, abnormal gait, tremors, abnormal respiration, paralysis, and spasticity. The onset of the adverse event is defined as the week post-dosing when the dysfunction was first recorded. If no adverse event was achieved, there is no onset (−). The onset of adverse events typically correlates with a failure to thrive as defined by a lack of body weight gain/maintenance similar to PBS-treated animals. Similar tolerability assessments were described in Oestergaard et al., Nucleic Acids Res., 2013 November, 41(21), 9634-9650 and Southwell et al., Mol Ther., 2014 December, 22(12), 2093-2106.

At the end of the study, the rats were sacrificed and tissues were collected. Histopathology was performed on sections of cerebellum using calbindin stain. Purkinje cell loss was observed in calbindin stained cerebellum sections as indicated in the table below. Cerebellum and spinal cord were also evaluated using an antibody specific for modified oligonucleotides. Animals demonstrating no oligonucleotide uptake were excluded from histopathology analysis. Histology was not completed for animals that were sacrificed early due to adverse events. Additionally, cortical GFAP, a marker of astrogliosis (Abdelhak, et al., Scientific Reports, 2018, 8, 14798), was measured using RT-PCR, and average elevations >2-fold are noted below.

TABLE 50
Long-term tolerability in rats at 3 mg dose

| | Adverse | Purkinje | |
|---|---|---|---|
| | event | cell | |
| | onset, | loss (# | Cortex |
| | weeks | animals | GFAP |
| | post- | with | mRNA |
| | treatment, | loss/# | >2-fold |
| Compound | individual | animals | PBS |
| Number | animals | tested) | Control |
| PBS | No | Not observed | N/A |
| 396442 | 6, 6, 2 | 2/3 | Yes |
| 819735 | 4, 6, 6, — | 1/4 | Yes |
| 1263789 | —, —, — | 0/3 | No |
| 1287717 | —, —, —, —, —, —, —, — | 0/8 | No |
| 1287745 | —, —, —, —, —, —, — | 0/7 | No |
| 1358996 | —, —, —, — | 0/4 | No |
| 1263783 | —, —, —, — | 0/4 | No |
| 1263785 | —, —, — | 0/3 | No |
| 1263787 | —, —, —, — | 0/4 | No |
| 1263800 | —, — | 0/2 | No |
| 1263802 | —, —, — | 0/3 | No |
| 1263806 | —, —, — | 0/3 | No |
| 1263808 | —, —, — | 0/3 | No |
| 1263810 | —, —, — | 0/3 | No |

### Example 8: Tolerability and Pharmacokinetics of Modified Oligonucleotides in Non-Human Primates, Single or Repeat Dosing

Cynomolgus monkeys are treated with modified oligonucleotides to determine the local and systemic tolerability and pharmacokinetics of the modified oligonucleotides. Each group receives either artificial CSF or modified oligonucleotide as a single intrathecal lumbar bolus dose injection (IT), or, for repeat-dosing groups, an IT bolus dose on day 1 of the study, followed by IT bolus doses at later time points. Tissues are collected 1 week after the final injection.

In a single dose study, monkeys are administered a single dose of modified oligonucleotide and tolerability is assessed. Representative doses for single-dose studies in adult cynomolgus monkeys include 1 mg, 3 mg, 7 mg, and 35 mg.

In a repeat-dosing study, monkeys are administered an IT bolus dose on day 1 of the study, followed by weekly (e.g., days 8, 15, and 22 for a four-week study) or monthly (e.g., days 29, 57, and 84 for a 13 week study) IT bolus dosing. Representative doses for repeat-dose studies in adult cynomolgus studies include 1 mg, 3 mg, 7 mg, and 35 mg. Assessment of tolerability is based on clinical observations, body weights, food consumption, physical and neurological examinations including sensorimotor reflexes, cerebral reflexes and spinal reflexes, coagulation, hematology, clinical chemistry (blood and cerebral spinal fluid (CSF)), cell count, and anatomic pathology evaluations. Complete necropsies are performed with a recording of any macroscopic abnormality. Organ weights are taken and microscopic examinations are conducted. Blood is collected for complement analysis. In addition, blood, CSF, and tissues (at necropsy) are collected for toxicokinetic evaluations.

Tolerability of modified oligonucleotides is analyzed in brain and spinal cord tissue by measuring Aif1 and Gfap levels in cynomolgus monkeys treated with the modified oligonucleotide or the control. Brain and spinal cord samples are collected and flash frozen in liquid nitrogen and stored frozen (−60° C. to −90° C.). At time of sampling, 2 mm biopsy punches are used to collect samples from frozen tissues for RNA analysis. Punches are taken from multiple brain and spinal cord regions.

### Example 9: Phase Ia Human Clinical Trial with Compound No. 1263789, 1287717, 1287745, or 1358996

Safety, tolerability, pharmacokinetics, pharmacodynamics and efficacy of modified oligonucleotide complementary to human SMN2 is evaluated in a clinical trial setting. Single and/or multiple doses of modified oligonucleotide are evaluated in patients with confirmed SMA, such as Type I SMA, Type II SMA, Type III SMA, or Type IV SMA.

Patient safety is monitored closely during the study. Safety and tolerability evaluations include: physical examination and standard neurological assessment (including fundi), vital signs (HR, BP, orthostatic changes, weight), ECG, AEs and concomitant medications, Columbia Suicide Severity Rating Scale (C-SSRS), CSF safety labs (cell counts, protein, glucose), plasma laboratory tests (clinical chemistry, hematology), and urinalysis.

Efficacy evaluations are selected that are age and Type appropriate and include, for example, the Hammersmith Motor Function Scale—Expanded (HFMSE), which is a reliable and validated tool used to assess motor function in children with SMA; the Pediatric Quality of Life Inventory (PedsQL™) Measurement 4.0 Generic Core Scale; the Pediatric Quality of Life Inventory 3.0 Neuromuscular Modules; the Compound Muscle Action Potential (CMAP); the Motor Unit Number Estimation (MUNE); the Upper Limb Module (ULM); and the 6-Minute Walk Test (6MWT) (Darras, et al., Neurology, 2019, 92: e2492-e2506).

### Example 10: Design of Modified Oligonucleotides Complementary to a Human SMN2 Nucleic Acid

Modified oligonucleotides complementary to a human SMN2 nucleic acid were designed and synthesized as indicated in the tables below.

Each modified oligonucleotide listed in the tables below is 100% complementary to SEQ ID NO: 1 (GENBANK Accession No. NT_006713.14 truncated from nucleotides 19939708 to 19967777). “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence.

The modified oligonucleotides in the table below are 18 nucleosides in length. Each nucleoside comprises either a 2′-MOE sugar moiety or a 2′-NMA sugar moiety. The sugar motif for each modified oligonucleotide is provided in the Sugar Motif column, wherein each ‘e’ represents a 2′-MOE sugar moiety, and each ‘n’ represents a 2′-NMA sugar moiety. Each internucleoside linkage is either a phosphorothioate internucleoside linkage, a phosphodiester internucleoside linkage, a methoxypropyl phosphonate internucleoside linkage, or a mesyl phosphoramidate (MsP) internucleoside linkage. The internucleoside linkage motif for each modified oligonucleotide, is provided in the Internucleoside Linkage Motif column, wherein each ‘s’ represents a phosphorothioate internucleoside linkage, each ‘o’ represents a phosphodiester internucleoside linkage, each ‘x’ represents a methoxypropyl phosphonate internucleoside linkage, and each ‘z’ represents a mesyl phosphoramidate (MsP) internucleoside linkage. Each cytosine is a 5-methyl cytosine. Modified oligonucleotide 449320 has been previously described in WO2015/161170 A2.

TABLE 51
MOE and NMA modified oligonucleotides with mixed PO/PS, PO/MsP,
uniform MsP, or PS/MOP internucleoside linkages

| | | | | SEQ ID | SEQ ID | |
|---|---|---|---|---|---|---|
| | | | Internucleoside | No: 1 | No: 1 | SEQ |
| Compound | | Sugar Motif | Linkage Motif | Start | Stop | ID |
| Number | Sequence (5′ to 3′) | (5′ to 3′) | (5′ to 3′) | Site | Site | No. |
| 449320 | TCACTTTCATAATGCTGG | eeeeeeeeeeeeeeeeee | ssoooooooooooooss | 27062 | 27079 | 23 |
| 1287723 | TCACTTTCATAATGCTGG | nnnnnnnnnnnnnnnenn | sssssssssssssssxs | 27062 | 27079 | 23 |
| 1287724 | TCACTTTCATAATGCTGG | nnnnnnnnnnnnnnnnen | ssssssssssssssssx | 27062 | 27079 | 23 |
| 1287727 | CACTTTCATAATGCTGGC | nnnnnnnnnnnnnnnnen | ssssssssssssssssx | 27061 | 27078 | 21 |
| 1405549 | TCACTTTCATAATGCTGG | eeeeeeeeeeeeeeeeee | zzzzzzzzzzzzzzzzz | 27062 | 27079 | 23 |
| 1405552 | TCACTTTCATAATGCTGG | eeeeeeeeeeeeeeeeee | ssssssssssszzzzzz | 27062 | 27079 | 23 |
| 1405553 | TCACTTTCATAATGCTGG | eeeeeeeeeeeeeeeeee | ssssszzzzzzssssss | 27062 | 27079 | 23 |
| 1545359 | TCACTTTCATAATGCTGG | nnnnnnnnnnnnnnnnnn | ssoooooooooooooss | 27062 | 27079 | 23 |
| 1547773 | TCACTTTCATAATGCTGG | eeeeeeeeeeeeeeeeee | zzooooooooooooozz | 27062 | 27079 | 23 |
| 1549028 | TCACTTTCATAATGCTGG | eeeeeeeeeeeeeeeeee | zzzzooooooooooozz | 27062 | 27079 | 23 |
| 1549029 | TCACTTTCATAATGCTGG | eeeeeeeeeeeeeeeeee | zzzzzzooooooooozz | 27062 | 27079 | 23 |
| 1549030 | TCACTTTCATAATGCTGG | eeeeeeeeeeeeeeeeee | zzzzzzzzooooooozz | 27062 | 27079 | 23 |

The modified oligonucleotides in the table below all consist of the sequence (from 5′ to 3′): TCACTTTCATAATGCTGG (SEQ ID NO: 23). Each modified oligonucleotide listed in the tables below is 100% complementary to SEQ ID NO: 1 (described herein above). “Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. The modified oligonucleotides in the table below are 18 nucleosides in length. Each nucleoside comprises either a 2′-MOE sugar moiety or a 2′-NMA sugar moiety. The sugar motif for each modified oligonucleotide is provided in the Sugar Motif column, wherein each ‘e’ represents a 2′-MOE sugar moiety, and each ‘n’ represents a 2′-NMA sugar moiety. Each internucleoside linkage is either a phosphorothioate internucleoside linkage, a phosphodiester internucleoside linkage, or a mesyl phosphoramidate (MsP) internucleoside linkage. The internucleoside linkage motif for each modified oligonucleotide, is provided in the Internucleoside Linkage Motif column, wherein each ‘s’ represents a phosphorothioate internucleoside linkage, each ‘o’ represents a phosphodiester internucleoside linkage, and each ‘z’ represents a mesyl phosphoramidate (MsP) internucleoside linkage. Each cytosine is a 5-methyl cytosine. The modified oligonucleotides in the table below are conjugated to a 6-palmitamidohexyl phosphate conjugate group attached to the 5′-OH of the oligonucleotide. The structure for the conjugate group is:

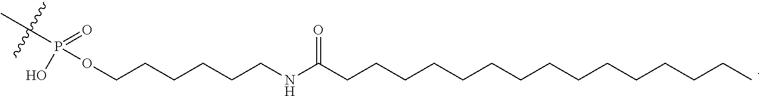 [SMILES: *P(=O)(O)OCCCCCCNC(=O)CCCCCCCCCCCCCCC]

TABLE 52
6-palmitamidohexyl conjugated MOE and NMA modified
oligonucleotides with mixed PO/PS, PO/MsP,
or uniform MsP internucleoside linkages

| | | | SEQ ID | SEQ ID | |
|---|---|---|---|---|---|
| | | Internucleoside | No: 1 | No: 1 | |
| Compound | Sugar Motif | Linkage Motif | Start | Stop | SEQ ID |
| Number | (5′ to 3′) | (5′ to 3′) | Site | Site | No. |
| 1545361 | eeeeeeeeeeeeeeeeee | ssoooooooooooooss | 27062 | 27079 | 23 |
| 1545362 | nnnnnnnnnnnnnnnnnn | ssoooooooooooooss | 27062 | 27079 | 23 |
| 1547772 | eeeeeeeeeeeeeeeeee | zzzzzzzzzzzzzzzzz | 27062 | 27079 | 23 |
| 1547774 | eeeeeeeeeeeeeeeeee | zzooooooooooooozz | 27062 | 27079 | 23 |
| 1549031 | eeeeeeeeeeeeeeeeee | zzzzooooooooooozz | 27062 | 27079 | 23 |
| 1549032 | eeeeeeeeeeeeeeeeee | zzzzzzooooooooozz | 27062 | 27079 | 23 |
| 1549033 | eeeeeeeeeeeeeeeeee | zzzzzzzzooooooozz | 27062 | 27079 | 23 |

The modified oligonucleotides in the table below all consist of the sequence (from 5′ to 3′): TCACTTTCATAATGCTGG (SEQ ID NO: 23), with a start site of 27062, and a stop site of 27079 on SEQ ID No: 1 (described herein above), wherein “start site” indicates the 5′-most nucleoside to which the modified oligonucleotide is complementary in the target nucleic acid sequence, and wherein “stop site” indicates the 3′-most nucleoside to which the modified oligonucleotide is complementary in the target nucleic acid sequence.

The modified oligonucleotides in the table below are 18 nucleosides in length. The sugar and internucleoside linkage motifs for each modified oligonucleotide are provided in the Sequence and Chemistry Notation column, wherein each subscript ‘n’ represents a 2′-NMA sugar moiety, each subscript ‘[DMA]’ represents a 2′-O—(N,N-dimethyl) acetamide moiety, each subscript ‘[NEA]’ represents a 2′-O-(N-ethyl) acetamide moiety, each subscript ‘[NPA]’ represents a 2′-O-(N-propyl) acetamide moiety, each subscript ‘[NcPA]’ represents a 2′O-(N-cyclopropyl) acetamide moiety, each subscript ‘[McPA]’ represents a 2′-O-(N-cyclopropylmethyl) acetamide moiety, and each subscript ‘s’ represents a phosphorothioate internucleoside linkage. Each cytosine is a 5-methyl cytosine, wherein the superscript ‘m’ before the cytosine residue (^{m}C) represents a 5-methyl cytosine. The structures for each of the sugars represented in the table below are:

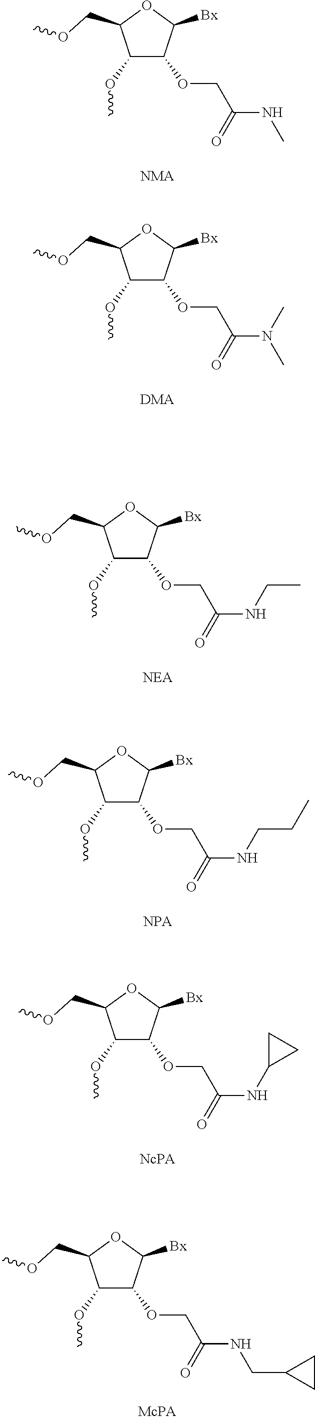

TABLE 53
NMA and NMA analog modified oligonucleotides with uniform PS internucleoside linkages

| Compound | | SEQ |
|---|---|---|
| Number | Sequence and Chemistry Notation (5′ to 3′) | ID No. |
| 1355763 | T_{[DMA]s}^{m}C_{[DMA]s}Aₙₛ^{m}C_{[DMA]s}T_{[DMA]s}T_{[DMA]s}^{m}C_{[DMA]s}AₙₛT_{[DMA]s}AₙₛAₙₛT_{[DMA]s}Gₙₛ^{m}C_{[DMA]s}T_{[DMA]s}GₙₛGₙ | 23 |
| 1359463 | T_{[NEA]s}^{m}C_{[NEA]s}Aₙₛ^{m}C_{[NEA]s}T_{[NEA]s}T_{[NEA]s}T_{[NEA]s}^{m}C_{[NEA]s}AₙₛT_{[NEA]s}AₙₛAₙₛT_{[NEA]s}Gₙₛ^{m}C_{[NEA]s}T_{[NEA]s}GₙₛGₙ | 23 |
| 1358995 | T_{[NPA]s}^{m}C_{[NPA]s}Aₙₛ^{m}C_{[NPA]s}T_{[NPA]s}T_{[NPA]s}T_{[NPA]s}^{m}C_{[NPA]s}AₙₛT_{[NPA]s}AₙₛAₙₛT_{[NPA]s}Gₙₛ^{m}C_{[NpA]s}T_{[NpA]s}GₙₛGₙ | 23 |
| 1355776 | T_{[NcPA]s}^{m}C_{[NcPA]s}Aₙₛ^{m}C_{[NcPA]s}T_{[NcPA]s}T_{[NcPA]s}T_{[NcPA]s}^{m}C_{[NcPA]s}AₙₛT_{[NcPA]s}AₙₛAₙₛT_{[NcPA]s}Gₙₛ^{m}C_{[NcPA]s}T_{[NcPA]s}GₙₛGₙ | 23 |
| 1355777 | T_{[McPA]s}^{m}C_{[McPA]s}Aₙₛ^{m}C_{[McPA]s}T_{[McPA]s}T_{[McPA]s}T_{[McPA]s}^{m}C_{[McPA]s}AₙₛT_{[McPA]s}AₙₛAₙₛT_{[McPA]s}Gₙₛ^{m}C_{[McPA]s}T_{[McPA]s}GₙₛGₙ | 23 |

### Example 11: Activity of Modified Oligonucleotides Complementary to Human SMN2 in Transgenic Mice, Single Dose (35 μg)

Activity of selected modified oligonucleotides described above was tested in human SMN2 transgenic mice essentially as described above in Example 2.

### Treatment

The transgenic mice were divided into groups of 4 mice each. Each mouse received a single ICV bolus of modified oligonucleotide at doses as indicated in the tables below. A group of 4 mice received PBS as a negative control. Two weeks post treatment, mice were sacrificed and RNA was extracted from coronal brain and spinal cord for real-time qPCR analysis of SMN2 RNA. Results are presented as fold change in RNA levels relative to PBS control, normalized to total SMN2 levels. ED₅₀ for exon inclusion (exon 7+) was calculated in GraphPad Prism 7 using nonlinear regression, 4-parameter dose response curve [Y=Bottom+(Top−Bottom)/(1+(10\hat{ }log EC50/X)\hat{ }HillSlope)].

### RNA Analysis

Two weeks post treatment, mice were sacrificed and RNA was extracted from cortical brain tissue and spinal cord for real-time qPCR analysis of SMN2 RNA. Primer probe set hSMN2vd #4_LTS00216_MGB was used to determine the amount of SMN2 RNA including exon 7 (exon 7+). Primer probe set hSMN2_Sumner68_PPS50481 was used to determine the amount of SMN2 RNA excluding exon 7 (exon 7). Total SMN2 RNA levels were measured using primer probe set hSMN2_LTS00935. Results are presented as fold change in RNA levels relative to PBS control, normalized to total SMN2 levels.

TABLE 54
Effect of modified oligonucleotides on human SMN2
RNA splicing in heterozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1 | 1 | 1 | 1 |
| 396443 | 35 | 3 | 0.6 | 3 | 0.5 |
| 1405549 | 35 | 1.6 | 0.9 | 1.2 | 0.9 |
| 1405552 | 35 | 3.2 | 0.6 | 2.1 | 0.7 |
| 1405553 | 35 | 2.4 | 0.7 | 1.7 | 0.8 |

TABLE 55
Effect of modified oligonucleotides on human SMN2 RNA
splicing in heterozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1 | 1 | 1 | N/A |
| 396443 | 35 | 2.7 | 0.5 | 3 | N/A |
| 1405549 | 35 | 1.5 | 0.8 | 1.9 | N/A |
| 1547772 | 35 | 1.8 | 0.8 | 1.8 | N/A |
| 1547773 | 35 | 1.6 | 0.9 | 1.9 | N/A |
| 1547774 | 35 | 1.6 | 1 | 1.6 | N/A |
| 1549028 | 35 | 1.5 | 1 | 1.5 | N/A |
| 1549029 | 35 | 1.4 | 1 | 1.7 | N/A |
| 1549030 | 35 | 1.4 | 1 | 1.4 | N/A |
| 1549031 | 35 | 1.7 | 0.8 | 1.6 | N/A |
| 1549032 | 35 | 1.4† | 0.9† | 1.7† | N/A |
| 1549033 | 35 | 1.4 | 1 | 1.3 | N/A |

†indicates fewer than four samples available

### Example 12: Activity of Modified Oligonucleotides Complementary to Human SMN2 in Transgenic Mice, Single Dose (15 μg)

Activity of selected modified oligonucleotides described above was tested in human SMN2 transgenic mice essentially as described above in Example 2.

### Treatment

The transgenic mice were divided into groups of 4 mice each. Each mouse received a single ICV bolus of modified oligonucleotide at doses as indicated in the tables below. A group of 4 mice received PBS as a negative control. Two weeks post treatment, mice were sacrificed and RNA was extracted from coronal brain and spinal cord for real-time qPCR analysis of SMN2 RNA. Results are presented as fold change in RNA levels relative to PBS control, normalized to total SMN2 levels. ED₅₀ for exon inclusion (exon 7+) was calculated in GraphPad Prism 7 using nonlinear regression, 4-parameter dose response curve [Y=Bottom+(Top−Bottom)/(1+(10\hat{ }log EC50/X)\hat{ }HillSlope)].

### RNA Analysis

Two weeks post treatment, mice were sacrificed and RNA was extracted from cortical brain tissue and spinal cord for real-time qPCR analysis of SMN2 RNA. Primer probe set hSMN2vd #4_LTS00216_MGB was used to determine the amount of SMN2 RNA including exon 7 (exon 7⁺). Primer probe set hSMN2_Sumner68_PPS50481 was used to determine the amount of SMN2 RNA excluding exon 7 (exon 7⁻). Total SMN2 RNA levels were measured using primer probe set hSMN2_LTS00935. Results are presented as fold change in RNA levels relative to PBS control, normalized to total SMN2 levels.

TABLE 56
Effect of modified oligonucleotides on human SMN2
RNA splicing in heterozygous transgenic mice

| Compound | Dose | CORTEX | | SPINAL CORD | |
|---|---|---|---|---|---|
| No. | (μg) | exon 7⁺ | exon 7⁻ | exon 7⁺ | exon 7⁻ |
| PBS | — | 1 | 1 | 1 | 1 |
| 443305 | 15 | 3.4 | 0.4 | 3.3 | 0.3 |
| 1287723 | 15 | 3.9 | 0.3 | 3.3 | 0.3 |
| 1287724 | 15 | 4.2 | 0.2 | 3.7 | 0.2 |
| 1287727 | 15 | 4.5 | 0.2 | 3.4 | 0.2 |

### Example 13: Activity of Modified Oligonucleotides Complementary to Human SMN2 in Transgenic Mice, Multiple Dose

Activity of selected modified oligonucleotides described above was tested in human SMN2 transgenic mice essentially as described above in Example 2.

### Treatment

The transgenic mice were divided into groups of 4 mice each. Each mouse received a single ICV bolus of modified oligonucleotide at multiple doses as indicated in the tables below. A group of 4 mice received PBS as a negative control. Two weeks post treatment, mice were sacrificed and RNA was extracted from coronal brain and spinal cord for real-time qPCR analysis of SMN2 RNA. Results are presented as fold change in RNA levels relative to PBS control, normalized to total SMN2 levels. ED₅₀ for exon inclusion (exon 7+) was calculated in GraphPad Prism 7 using nonlinear regression, 4-parameter dose response curve [Y=Bottom+(Top−Bottom)/(1+(10\hat{ }log EC50/X)\hat{ }HillSlope)].

### RNA Analysis

Two weeks post treatment, mice were sacrificed and RNA was extracted from cortical brain tissue and spinal cord for real-time qPCR analysis of SMN2 RNA. Primer probe set hSMN2vd #4_LTS00216_MGB was used to determine the amount of SMN2 RNA including exon 7 (exon 7+). Primer probe set hSMN2_Sumner68_PPS50481 was used to determine the amount of SMN2 RNA excluding exon 7 (exon 7). Total SMN2 RNA levels were measured using primer probe set hSMN2_LTS00935. Results are presented as fold change in RNA levels relative to PBS control, normalized to total SMN2 levels.

TABLE 57
Effect of modified oligonucleotides on human
SMN2 RNA splicing in heterozygous transgenic mice

| Compound | Dose | CORTEX | | ED50 | SPINAL CORD | | ED50 |
|---|---|---|---|---|---|---|---|
| No. | (μg) | exon 7+ | exon 7− | (μg) | exon 7+ | exon 7− | (μg) |
| PBS | — | 1 | 1 | — | 1 | 1 | — |
| 396443 | 10 | 2.1 | 0.7 | 26 | 1.9 | 0.8 | 22 |
| | 30 | 2.8 | 0.5 | | 2.6 | 0.7 | |
| | 100 | 3.2 | 0.3 | | 2.8 | 0.4 | |
| 449320 | 10 | 1.2 | 1.0 | >100 | 1.2 | 1.2 | >100 |
| | 30 | 1.5 | 1.0 | | 1.3 | 1.2 | |
| | 100 | 1.5 | 0.9 | | 1.3 | 0.9 | |
| 1545361 | 10 | 1.4 | 1.0 | >100 | 1.2 | 1.1 | >100 |
| | 30 | 1.8 | 1.1 | | 1.6 | 1.3 | |
| | 100 | 1.3 | 0.9 | | 1.4 | 0.9 | |
| 443305 | 10 | 2.4 | 0.6 | 14 | 2.6 | 0.5 | 9 |
| | 30 | 3.5 | 0.3 | | 3.0 | 0.4 | |
| | 100 | 3.7 | 0.1 | | 3.3 | 0.1 | |
| 1545359 | 10 | 1.4 | 1.0 | >100 | 1.4 | 1.1 | >100 |
| | 30 | 2.0 | 0.9 | | 1.7 | 1.1 | |
| | 100 | 2.3 | 0.6 | | 1.7 | 0.8 | |
| 1545362 | 10 | 1.4 | 0.9 | 95 | 1.3 | 1.1 | 51 |
| | 30 | 1.9 | 0.8 | | 2.4 | 1.0 | |
| | 100 | 2.7 | 0.5 | | 2.6 | 0.6 | |

TABLE 58
Effect of modified oligonucleotides on human
SMN2 RNA splicing in heterozygous transgenic mice

| Compound | Dose | CORTEX | | ED50 | SPINAL CORD | | ED50 |
|---|---|---|---|---|---|---|---|
| No. | (μg) | exon 7+ | exon 7− | (μg) | exon 7+ | exon 7− | (μg) |
| PBS | — | 1 | 1 | — | 1 | 1 | — |
| 1263789 | 3 | 1.5 | 0.9 | 35 | 1.6 | 0.8 | 16 |
| | 10 | 1.9 | 0.8 | | 2.2 | 0.6 | |
| | 30 | 2.7 | 0.6 | | 2.9 | 0.5 | |
| | 100 | 3.8 | 0.3 | | 3.5 | 0.3 | |
| | 300 | 4.3 | 0.2 | | 3.9 | 0.2 | |
| 1287703 | 3 | 1.5 | 0.7 | 43 | 1.5 | 0.8 | 28 |
| | 10 | 1.6 | 0.7 | | 2.0 | 0.7 | |
| | 30 | 2.7 | 0.5 | | 2.6 | 0.5 | |
| | 100 | 3.7 | 0.3 | | 3.2 | 0.4 | |
| | 300 | 4.0 | 0.2 | | 3.5 | 0.2 | |
| 1287717 | 3 | 1.4 | 0.8 | 31 | 1.4 | 0.9 | 30 |
| | 10 | 1.7 | 0.7 | | 1.8 | 0.7 | |
| | 30 | 3.1 | 0.4 | | 2.4† | 0.5† | |
| | 100 | 3.8 | 0.3 | | 3.3 | 0.3 | |
| | 300 | 4.3 | 0.2 | | 3.8 | 0.2 | |
| 1318768 | 3 | 1.3 | 0.8 | 49 | 1.2 | 0.8 | 32 |
| | 10 | 1.8 | 0.7 | | 1.8 | 0.8 | |
| | 30 | 2.4 | 0.6 | | 2.4 | 0.6 | |
| | 100 | 3.5 | 0.3 | | 3.3 | 0.4 | |
| | 300 | 4.1 | 0.2 | | 3.9 | 0.2 | |
| 1287731 | 3 | 1.5 | 0.8 | 29 | 1.5 | 1.0 | 13 |
| | 10 | 2.0 | 0.6 | | 2.4 | 0.6 | |
| | 30 | 2.7 | 0.4 | | 3.2 | 0.4 | |
| | 100 | 4.3 | 0.2 | | 3.8 | 0.2 | |
| | 300 | 4.2 | 0.1 | | 3.7 | 0.1 | |
| 1287735 | 3 | 1.7 | 0.7 | 22 | 1.5 | 0.8 | 15 |
| | 10 | 2.4 | 0.6 | | 2.2 | 0.6 | |
| | 30 | 3.0 | 0.3 | | 3.1 | 0.3 | |
| | 100 | 4.0 | 0.2 | | 3.5 | 0.2 | |
| | 300 | 4.2 | 0.1 | | 3.9 | 0.1 | |
| 1287745 | 3 | 1.5 | 0.7 | 35 | 1.4 | 0.8 | 13 |
| | 10 | 2.0 | 0.6 | | 2.4 | 0.6 | |
| | 30 | 2.9 | 0.4 | | 3.2 | 0.4 | |
| | 100 | 3.7 | 0.2 | | 3.6 | 0.2 | |
| | 300 | 3.7 | 0.2 | | 3.8 | 0.2 | |
| 396443 | 3 | 1.7 | 0.7 | 47 | 1.5 | 0.9 | 22 |
| | 10 | 1.4 | 0.7 | | 1.6 | 0.8 | |
| | 30 | 2.8 | 0.5 | | 3.1 | 0.4 | |
| | 100 | 3.3 | 0.4 | | 3.3 | 0.5 | |
| | 300 | 4.3 | 0.2 | | 4.0 | 0.2 | |

†indicates fewer than four samples available

TABLE 59
Effect of modified oligonucleotides on human
SMN2 RNA splicing in heterozygous transgenic mice

| Compound | Dose | CORTEX | | ED50 | SPINAL CORD | | ED50 |
|---|---|---|---|---|---|---|---|
| No. | (μg) | exon 7+ | exon 7− | (μg) | exon 7+ | exon 7− | (μg) |
| PBS | — | 1 | 1 | — | 1 | 1 | — |
| 396443 | 3 | 1.1 | 0.9 | 39 | 1.2 | 0.9 | 33 |
| | 30 | 2.0 | 0.6 | | 2.3 | 0.5 | |
| | 100 | 2.3 | 0.4 | | 2.7 | 0.3 | |
| 443305 | 3 | 1.4 | 0.7 | 13 | 1.6 | 0.7 | 10 |
| | 30 | 2.4 | 0.3 | | 3.0 | 0.2 | |
| | 100 | 2.9 | 0.2 | | 3.1 | 0.2 | |
| 1355763 | 3 | 1.1 | 0.8 | 54 | 1.1 | 0.8 | 45 |
| | 30 | 1.7 | 0.6 | | 2.1 | 0.5 | |
| | 100 | 2.4 | 0.4 | | 2.6 | 0.3 | |
| 1359463 | 3 | 1.3 | 0.8 | 18 | 1.3 | 0.7 | 19 |
| | 30 | 2.3 | 0.3 | | 2.7 | 0.3 | |
| | 100 | 2.9 | 0.2 | | 2.7 | 0.2 | |
| 1358995 | 3 | 1.2 | 0.8 | 45 | 1.0 | 0.8 | 59 |
| | 30 | 2.0 | 0.4 | | 1.8 | 0.4 | |
| | 100 | 2.2 | 0.4 | | 2.6 | 0.3 | |
| 1355776 | 3 | 1.1 | 0.8 | 25 | 1.3 | 0.7 | 24 |
| | 30 | 2.2 | 0.4 | | 2.4 | 0.4 | |
| | 100 | 2.6 | 0.3 | | 2.9 | 0.3 | |
| 1355777 | 3 | 1.0 | 0.9 | 107 | 0.9 | 0.9 | 72 |
| | 30 | 1.6 | 0.7 | | 1.8 | 0.6 | |
| | 100 | 1.7 | 0.5 | | 2.2 | 0.6 | |

### Example 15: Tolerability of Modified Oligonucleotides Complementary to SMN2 in Wild-Type Mice

Modified oligonucleotides described above were tested in wild-type female C57/B16 mice to assess the tolerability of the oligonucleotides. Wild-type female C57/B16 mice each received a single ICV dose of 700 μg of modified oligonucleotide listed in the table below. Each treatment group consisted of 4 mice. A group of 4 mice received PBS as a negative control for each experiment (identified in separate tables below). At 3 hours post-injection, mice were evaluated according to seven different criteria. The criteria are (1) the mouse was bright, alert, and responsive; (2) the mouse was standing or hunched without stimuli; (3) the mouse showed any movement without stimuli; (4) the mouse demonstrated forward movement after it was lifted; (5) the mouse demonstrated any movement after it was lifted; (6) the mouse responded to tail pinching; (7) regular breathing. For each of the 7 criteria, a mouse was given a subscore of 0 if it met the criteria and 1 if it did not (the functional observational battery score or FOB). After all 7 criteria were evaluated, the scores were summed for each mouse and averaged within each treatment group. The results are presented in the tables below.

TABLE 60
Tolerability scores in
mice at 700 μg dose

| Compound | 3 hr |
|---|---|
| Number | FOB |
| PBS | 0.00 |
| 1287723 | 2.00 |
| 1287724 | 1.00 |
| 1287727 | 2.00 |

1.-31. (canceled)

32. A modified oligonucleotide according to the following chemical structure: 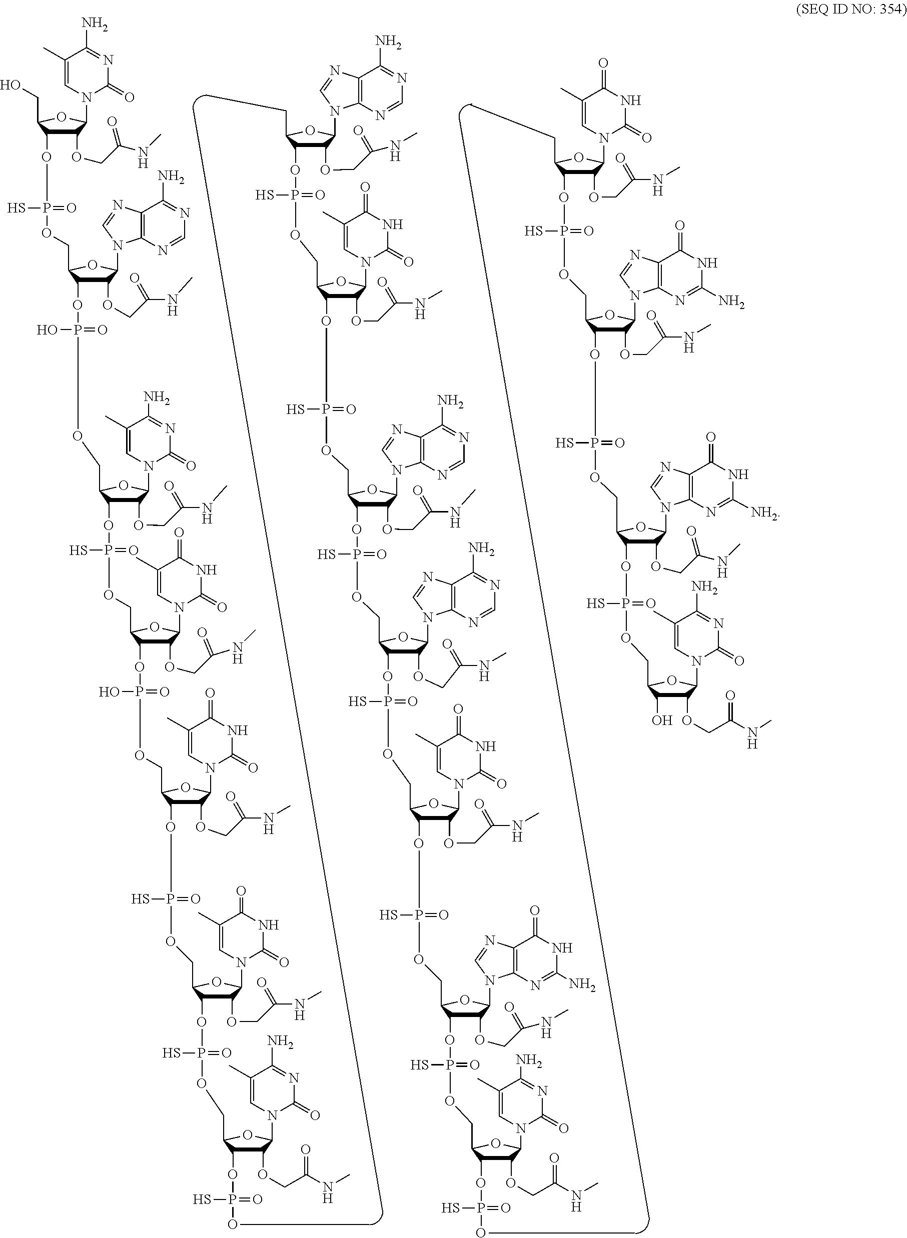
or a salt thereof.

33. A modified oligonucleotide according to the following chemical structure:

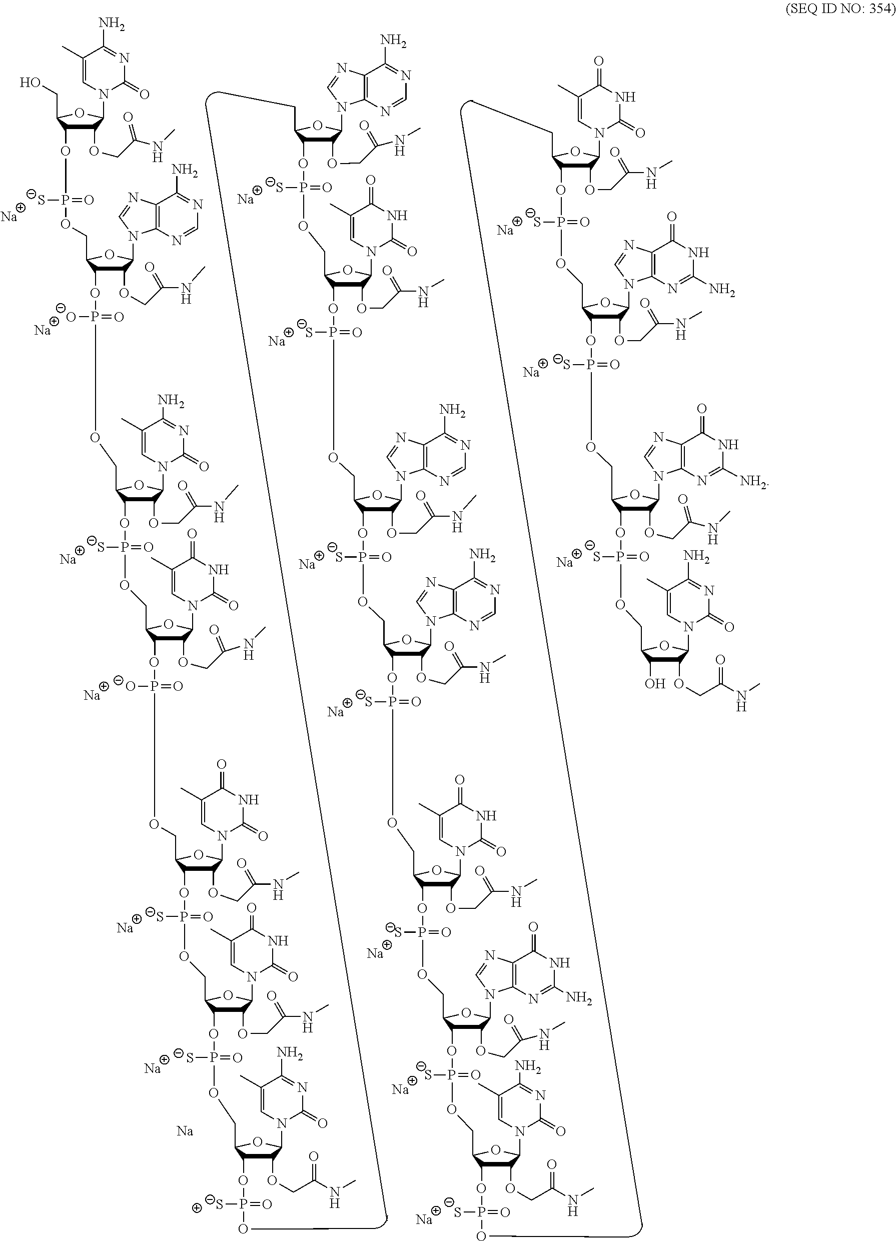

34. A modified oligonucleotide according to the following chemical notation: ^{m}Cₙₛ Aₙₒ ^{m}Cₙₛ Tₙₒ Tₙₛ Tₙₛ ^{m}Cₙₛ Aₙₛ Tₙₛ Aₙₛ Aₙₛ Tₙₛ Gₙₛ ^{m}Cₙₛ Tₙₛ Gₙₛ Gₙₛ ^{m}Cₙ (SEQ ID NO: 52), wherein:
A=an adenine nucleobase,
^{m}C=a 5-methyl cytosine nucleobase,
G=a guanine nucleobase,
T=a thymine nucleobase,
n=a 2′-NMA sugar moiety,
s=a phosphorothioate internucleoside linkage, and
o=a phosphodiester internucleoside linkage.

35. A chirally enriched population of modified oligonucleotides of claim 32, wherein the population is enriched for modified oligonucleotides comprising at least one particular phosphorothioate internucleoside linkage having a particular stereochemical configuration.

36. A chirally enriched population of modified oligonucleotides of claim 33, wherein the population is enriched for modified oligonucleotides comprising at least one particular phosphorothioate internucleoside linkage having a particular stereochemical configuration.

37. A chirally enriched population of modified oligonucleotides of claim 34, wherein the population is enriched for modified oligonucleotides comprising at least one particular phosphorothioate internucleoside linkage having a particular stereochemical configuration.

38. A pharmaceutical composition comprising the modified oligonucleotide of claim 32, and a pharmaceutically acceptable diluent or carrier.

39. A pharmaceutical composition comprising the modified oligonucleotide of claim 33, and a pharmaceutically acceptable diluent or carrier.

40. A pharmaceutical composition comprising the modified oligonucleotide of claim 34, and a pharmaceutically acceptable diluent or carrier.

41. A pharmaceutical composition comprising the population of modified oligonucleotides of claim 35, and a pharmaceutically acceptable diluent or carrier.

42. A pharmaceutical composition comprising the population of modified oligonucleotides of claim 36, and a pharmaceutically acceptable diluent or carrier.

43. A pharmaceutical composition comprising the population of modified oligonucleotides of claim 37, and a pharmaceutically acceptable diluent or carrier.

44. A method of treating spinal muscular atrophy (SMA) comprising administering to a human subject in need thereof a therapeutically effective amount of a pharmaceutical composition comprising the modified oligonucleotide of claim 32.

45. A method of treating spinal muscular atrophy (SMA) comprising administering to a human subject in need thereof a therapeutically effective amount of a pharmaceutical composition comprising the modified oligonucleotide of claim 33.

46. A method of treating spinal muscular atrophy (SMA) comprising administering to a human subject in need thereof a therapeutically effective amount of a pharmaceutical composition comprising the modified oligonucleotide of claim 34.

47. A method of increasing SMN2 RNA including exon 7 comprising contacting a cell, tissue, or organ with the modified oligonucleotide of claim 32.

48. A method of increasing SMN2 RNA including exon 7 comprising contacting a cell, tissue, or organ with the modified oligonucleotide of claim 33.

49. A method of increasing SMN2 RNA including exon 7 comprising contacting a cell, tissue, or organ with the modified oligonucleotide of claim 34.
